(19) United States
(12) Patent Application Publication
(10) Pub. No.: US 2005/0084489 A1
(43) Pub. Date: Apr. 21, 2005

(54) Methods of preventing or treating disorders by administering and integrin alphanubeta3 antagonist in combination with an HMG-CoA reductase inhibitor or a bisphosphonate

(76) Inventors: Ronald L. Wilder, Derwood, MD (US); Su-Yau Mao, Gaithersburg, MD (US)

Correspondence Address: JOHNATHAN KLEIN-EVANS, ONE MEDIMMUNE WAY, GAITHERSBURG, MD, 20878, US

(21) Appl. No.: 10/379,145

(22) Filed: Mar. 4, 2003

Related U.S. Application Data
(60) Provisional application No. 60/361,859, filed on Mar. 4, 2002.
(60) Provisional application No. 60/370,398, filed on Apr. 5, 2002.
(60) Provisional application No. 60/444,265, filed on Jan. 30, 2003.
(60) Provisional application No. 60/444,156, filed on Jan. 30, 2003.

(51) Int. Cl. A61K 39/395; A61K 31/66

(52) U.S. Cl. 424/144.1; 514/102

(57) ABSTRACT

The present invention provides methods of preventing, treating, managing or ameliorating disorders utilizing an integrin αᵥβ₃ antagonist in combination with an HMG-CoA reductase inhibitor and/or a bisphosphonate. The present invention also encompasses methods of preventing, treating, managing or ameliorating disorders utilizing an integrin αᵥβ₃ antagonist in combination with an HMG-CoA reductase inhibitor and/or a bisphophonate, in further combination with another therapy (e.g., another prophylactic or therapeutic agent or treatment) which is not an integrin αᵥβ₃ antagonist, an HMG-CoA reductase inhibitor, or a bisphosphonate. In particular, the present invention provides methods of preventing, treating, managing or ameliorating inflammatory diseases, autoimmune disorders, disorders associated with aberrant expression and/or activity of integrin αᵥβ₃, disorders associated with abnormal bone metabolism, disorders associated with aberrant angiogenesis and cancers, or conditions associated therewith, utilizing an antibody that immunospecifically binds to integrin αᵥβ₃ (e.g., VITAXIN®) in combination with an HMG-CoA reductase inhibitor and/or bisphosphonate, and optionally in combination with another therapy (e.g., another prophylactic or therapeutic agent or treatment) which is not an integrin αᵥβ₃ antagonist, an HMG-CoA reductase inhibitor, or a bisphosphonate. The present also invention encompasses compositions and articles of manufacture for use in preventing, treating, managing or ameliorating inflammatory diseases, autoimmune disorders, disorders associated with aberrant expression and/or activity of integrin αᵥβ₃, disorders associated with abnormal bone metabolism, disorders associated with aberrant angiogenesis and cancers, or conditions associated therewith.

The present application claims priority to U.S. provisional application Ser. No. 60/361,859 filed Mar. 4, 2002, U.S. provisional application Ser. No. 60/370,398 filed Apr. 5, 2002, U.S. provisional application Ser. No. 60/444,265 filed Jan. 30, 2003, and U.S. provisional application Ser. No. 60/444,156, filed Jan. 30, 2003, each of which is hereby incorporated by reference in its entirety.

### 1. FIELD OF THE INVENTION

The present invention provides methods of preventing, treating, managing or ameliorating a disease or disorder (e.g., an inflammatory disease, an autoimmune disorder, a disorder associated with aberrant expression and/or activity of integrin αᵥβ₃, a disorder associated with abnormal bone metabolism, a disorder associated with aberrant angiogenesis and/or cancer) or one or more conditions or symptoms associated therewith, utilizing an integrin αᵥβ₃ antagonist in combination with an HMG-CoA reductase inhibitor and/or a bisphosphonate. The present invention also encompasses methods of preventing, treating, managing or ameliorating a disease or disorder (e.g., an inflammatory disease, an autoimmune disorder, a disorder associated with aberrant expression and/or activity of integrin αᵥβ₃, a disorder associated with abnormal bone metabolism, a disorder associated with aberrant angiogenesis or cancer) or one or more conditions or symptoms associated therewith, utilizing an integrin αᵥβ₃ antagonist in combination with an HMG-CoA reductase inhibitor and/or a bisphophonate, and another therapy (e.g., another prophylactic or therapeutic agent) which does not comprise an integrin αᵥβ₃ antagonist, an HMG-CoA reductase inhibitor, or a bisphosphonate. In particular, the present invention provides methods of preventing, treating, managing or ameliorating a disease or disorder (e.g., an inflammatory disease, an autoimmune disorder, a disorder associated with aberrant expression and/or activity of integrin αᵥβ₃, a disorder associated with abnormal bone metabolism, a disorder associated with aberrant angiogenesis or cancer) or one or more conditions or symptoms associated therewith, utilizing an antibody that immunospecifically binds to integrin αᵥβ₃ (e.g., VITAXIN™) in combination with an HMG-CoA reductase inhibitor and/or bisphosphonate, and optionally in combination with another therapy (e.g., another prophylactic or therapeutic agent) which does not comprise an integrin αᵥβ₃ antagonist, an HMG-CoA reductase inhibitor, or a bisphosphonate. The present also invention encompasses compositions and articles of manufacture for use in preventing, treating, managing or ameliorating a disease or disorder (e.g., an inflammatory disease, an autoimmune disorder, a disorder associated with aberrant expression and/or activity of integrin αᵥβ₃, a disorder associated with abnormal bone metabolism, a disorder associated with aberrant angiogenesis or cancer) or one or more conditions or symptoms associated therewith.

### 2. BACKGROUND OF THE INVENTION

2.1 Cancer

A neoplasm, or tumor, is a neoplastic mass resulting from abnormal uncontrolled cell growth which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W.B. Saunders Co., Philadelphia, pp. 68-122). Cancer can arise in many sites of the body and behave differently depending upon its origin. Cancerous cells destroy the part of the body in which they originate and then spread to other part(s) of the body where they start new growth and cause more destruction.

Additionally, cancer cells can spread, metastasizing e.g., to bone, and producing chemicals that influence the activity of osteoclasts and osteoblasts, upsetting their normal balance. Metastatic bone diseases are more commonly associated with certain types of cancer, such as breast cancer (affecting more than half of women with breast cancer during the course of their disease). These metastases commonly result in small holes in the bone due to overactivity of the osteoclasts, increasing the likelihood of skeletal events and bone pain.

More than 1.2 million Americans develop cancer each year. Cancer is the second leading case of death in the United States and if current trends continue, cancer is expected to be the leading cause of the death by the year 2010. Lung and prostate cancer are the top cancer killers for men in the United States. Lung and breast cancer are the top cancer killers for women in the United States. One in two men in the United States will be diagnosed with cancer at some time during his lifetime. One in three women in the United States will be diagnosed with cancer at some time during her lifetime.

Currently, cancer therapy may involve surgery, chemotherapy, hormonal therapy and/or radiation treatment to eradicate neoplastic cells in a patient (see, for example, Stockdale, 1998, “Principles of Cancer Patient Management”, in Scientific American: Medicine, vol. 3, Rubenstein and Federman, eds., Chapter 12, Section IV). Recently, cancer therapy could also involve biological therapy or immunotherapy. All of these approaches pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and although it can be effective, is often used to prevent or delay recurrence of cancer after other treatments have removed the majority of the cancer cells. Biological therapies/immunotherapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for treatment of cancer. A significant majority of cancer chemotherapeutics act by inhibiting DNA synthesis, either directly, or indirectly by inhibiting the biosynthesis of the deoxyribonucleotide triphosphate precursors, to prevent DNA replication and concomitant cell division (see, for example, Gilman et al., Goodman and Gilman's: The Pharmacological Basis of Therapeutics, Eighth Ed. (Pergamom Press, New York, 1990)). These agents, which include alkylating agents, such as nitrosourea, anti-metabolites, such as methotrexate and hydroxyurea, and other agents, such as etoposides, campathecins, bleomycin, doxorubicin, daunorubicin, etc., although not necessarily cell cycle specific, kill cells during S phase because of their effect on DNA replication. Other agents, specifically colchicine and the vinca alkaloids, such as vinblastine and vincristine, interfere with microtubule assembly resulting in mitotic arrest. Chemotherapy protocols generally involve administration of a combination of chemotherapeutic agents to increase the efficacy of treatment.

Despite the availability of a variety of chemotherapeutic agents, chemotherapy has many drawbacks (see, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy causes significant, and often dangerous, side effects, including severe nausea, bone marrow depression, immunosuppression, etc. Additionally, even with the administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even those agents that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

There is a significant need for alternative cancer treatments, particularly for the treatment of cancer that has proved refractory to standard cancer treatments, such as surgery, radiation therapy, chemotherapy, and hormonal therapy. Further, it is uncommon for cancer to be treated by only one method. Thus, there is a need for the development of new therapeutic agents for the treatment of cancer and new, more effective, therapy combinations for the treatment of cancer.

2.2 Bone Metabolism Disorders

The skeleton is a metabolically active organ that undergoes continuous remodeling throughout life. In normal bone, two different types of cells found in bone, namely osteoclasts and osteoblasts, work together to maintain a healthy balance. Osteoclasts destroy old bone and osteoblasts deposit new minerals and build new bone. When bone loss in a subject outweighs bone formation, a bone metabolism disorder may be the cause.

Bone metabolism disorders include but are not limited to osteoporosis (including estrogen deficiency, immobilization, glucocorticoid-induced and senile osteoporosis), osteopenia, osteodystrophy, Paget's disease, myositis ossificans, Bechterew's disease, malignant hypercalcemia, metatstatic bone disease, periodontal disease, cholelithiasis, nephrolithiasis, urolithiasis, urinary calculus, hardening of the arteries (sclerosis), arthritis, rheumatoid arthritis, bone metastasis, bursitis, neuritis, and tetany.

Bone loss, including osteopenia (bone density is between 1 and 2.5 standard deviations below the young adult mean) and osteoporosis (bone density is greater than 2.5 standard deviations below the young adult mean), are major public health problems resulting in substantial morbidity and an estimated $14 billion in health costs annually. The number of American women suffering from osteopenia is estimated to be 15 million. The number of American women suffering from osteoporosis is estimated to be 8 million. For men, the numbers are 3 million and 2 million, respectively.

Current therapies for osteoporosis include peptides (e.g. calcitonin), bisphosphonates (e.g, alendronate), estrogen receptor modulators (e.g., estrogen, progestin, estradiol, droloxifene, raloxifene, and tamoxifene), androgen receptor modulators (Davis, S. 1999, J. Steroid Biochem. Mol. Biol. 69:177-184 “The Therapeutic Use of Androgens in Women” and Hansen, K. and Tho, S., 1998, Seminars in Repro. Endocrin. 16:129-134 “Androgens and Bone Health”), growth hormone secretagogues, cathepsin K inhibitors (U.S. Pat. No. 5,501,969, issued Mar. 3, 1996; and U.S. Pat. No. 5,736,357, issued Apr. 7, 1998), PPARγ activators (Okazaki, R. et al., 1999, Endocrinology 140:5060-5065), and inhibitors of the osteoclast proton ATPase (Farina, C. et al., 1999, DDT 4:163-172).

Treatments for other bone metabolism disorders include, but are not limited to calcium supplements, phosphate, aluminum hydroxide, aluminum carbonate gels, magnesium, vitamin D, active forms of Vitamin D (e.g., calcitriol (1,25 dihydroxycholecalciferol), vitamin D₂ (ergocalciferol), vitamin D₃ (cholecalciferol), calcium, lithium, glucocorticoids, plicamycin (mithramycin), gallium nitrate, hormones (e.g., estrogen, progestin and calcitonin), estrogen antagonists (e.g., tamoxifen), estrogen receptor modulators, androgen receptor modulators, cytotoxic or antiproliferative agents, matrix metalloproteinase inhibitors, inhibitors of epidermal-derived, fibroblast-derived, or platelet-derived growth factors, inhibitors of vascular endothelial growth factor (VEGF), antibodies to a growth factor or to a growth factor receptor, inhibitors of Flk-1/KDR, Flt-1 (VEGF receptors), inhibitors of Tck/Tie-2, or Tie-1 (tyrosine protein kinase receptors), cathepsin K inhibitors, inhibitors of osteoclast proton ATPase, inhibitors of urokinase plasminogen activator (u-PA), tumor-specific antibody-interleukin-2 fusion proteins, prenylation inhibitors, farnesyl transferase inhibitors, geranylgeranyl transferase inhibitors or dual famesyl/geranylgeranyl transferase inhibitors, parathyroid hormone or parathyroid hormone fragments (a non-limiting example is exogenous PTH analogue, 1-34 PTH), growth hormones, molecules disclosed in U.S. Pat. Nos. 6,472,402 and 6,482,411, renal dialysis, Evista® (raloxifene HCl), bisphosphonates (e.g., Actonel®, Aredia®, Didronel®, Fosamax® and Skelid®) and surgery.

Although several treatment options are available for bone metabolism disorders, there are multiple drawbacks including toxicity and poor long-term patient compliance. Thus, there is a need in the art for new, more effective therapies for the treatment of bone metabolism disorders.

2.3 Bisphosphonates

Bisphosphonates are a class of drugs which restrict the action of osteoclasts and result in a reduction in bone resorption. Bisphosphonates are used to ease bone pain, slow the spread and growth of metastatic cancers, strengthen bones in people with cancer that has affected the bone, reduce the risk of bone fracturing, and treat hypercalcemia (high levels of calcium in the blood). Accordingly, bisphosphonates are administered to patient populations with osteoporosis or cancer, or patient populations otherwise at risk for skeletal events (e.g., myeloma patients or secondary bone cancer patients, such as breast cancer patients where the cancer has spread to the bone or prostate cancer) to reduce the breakdown of bone, risk of fracture and discomfort (bone pain). See e.g., Pavlakis, N. and Stockler, M., 2002, “Bisphosphonates for Breast Cancer”, The Cochrane Library Issue 4; Homik, J. et al., 2002, “Bisphosphonates for Steroid Induced Osteoporosis” The Cochrane Library Issue 4; Wong, R. and Wiffen P., 2002, “Bisphosphonates for the Relief of Pain Secondary to Bone Metastases” The Cochrane Library Issue 4; Djulbegovic B., et al., 2002, “Bisphosphonates for Multiple Myeloma” Cochrane Library Issue 4, each of which is hereby incorporated by reference.

Side effects from biosphosphonate use include drop in calcium levels, change in kidney function, increased pain, bloating, gas, heartburn, nausea, headache, high temperature and chills, and dizziness. Bisphosphonates may also affect vision, leading to blurred vision, occular irritation, non-specific conjunctivitis (pain, epiphoria, photophobia), anterior uveitis, anterior scleritis, episcleritis, and periocular, lid and/or orbital edema. See Fraunfelder and Franufelder, 2003, “Scleritis and other ocular side effects associated with pamidronate disodium” Am. J. Ophthalmol. 135(2):219-22. A rare but more serious side effect from bisphosphonate use is liver damage. Symptoms of bisphosphonate-induced liver damage include yellow skin or eyes, dark urine, nausea and vomiting, or loss of appetite. Other side effects include muscle aches, tenderness and weakness. The toxicity and side effects of bisphosphonates underscore the need in the art for more effective therapies with less side effects for the treatment, prevention, management and amelioration of cancer and bone metabolism disorders, and conditions associated therewith.

2.4 HMG-CoA Reductase Inhibitors

HMG-CoA (3-hydroxy-3-methylglutaryl coenzyme A) reductase inhibitors (a.k.a. statins) are a class of drugs generally used to treat a number of diseases including elevated cholesterol levels. Their efficacy in lowering cholesterol levels is attributed to an ability to slow down the body's ability to make cholesterol in the blood. Studies have also shown that exposure to HMG-CoA reductase inhibitors is associated with a decreased risk of bone fractures in individuals age 50 years and older. Meier et al., 2000, “HMG-CoA Reductase Inhibitors and the Risk of Fractures” JAMA. 283:3205-3210. Additionally, HMG-CoA reductase inhibitors are indicated for use in patients with coronary artery disease and have been shown to be effective at lowering LDL levels and preventing cardiovascular events in angioplasty patients. See Serruys, P., et al., 2002, “Fluvastatin for Prevention of Cardiac Events Following Successful Percutaneous Coronary Intervention” JAMA 287: 3215-3222.

Common side effects from HMG-CoA reductase inhibitors include liver damage, stomach upset or pain, diarrhea, constipation, headache, dizziness, skin rash, muscle tenderness or soreness, unexplained muscle pain, general malaise, fatigue and weakness, and fever.

### 3. SUMMARY OF INVENTION

The present inventions encompasses treatment protocols for diseases in which an antagonist of integrin αᵥβ₃ is used in combination with one or more HMG-CoA reductase inhibitors and/or one or more bisphosphonates, and optionally another therapy (e.g., another prophylactic or therapeutic agent) that is not an integrin αᵥβ₃ antagonist, an HMG-CoA reductase inhibitor, and/or a bisphosphonate. The invention is based, in part, on the recognition that antagonists of integrin αᵥβ₃ potentiate and synergize with, enhance the effectiveness of, improve the tolerance of, and/or reduce the side effects caused by, other therapies, including, but not limited to, bisphophonates, HMG-CoA reductase inhibitors, anti-inflammatory therapies, autoimmune disorder therapies, therapies for disorders associated with aberrant expression and/or activity of integrin αᵥβ₃, therapies for disorders associated with abnormal bone metabolism, therapies for disorders associated with aberrant angiogenesis, and cancer therapies. The combination therapies of the invention have additive potency, an additive therapeutic effect or a synergistic effect. The combination therapies of the invention enable lower dosages of therapies to be utilized in conjunction with antagonists of integrin αᵥβ₃ for the prevention, management, treatment or amelioration of a disease and/or less frequent administration of such therapies to a subject with said disease to improve the quality of life of said subject and/or to achieve a prophylactic or therapeutic effect. The combination therapies of the invention enable lower dosages of one or more antagonists of integrin αᵥβ₃ and/or less frequent administration of dosages of one or more antagonists of integrin αᵥβ₃ to a subject with a disease to improve the quality of life of said subject and/or to achieve a prophylactic or therapeutic effect. Further, the combination therapies of the invention reduce or avoid unwanted or adverse side effects associated with the administration of current single agent therapies and/or existing combination therapies for diseases, which in turn improves patient compliance with the treatment protocol.

The present invention encompasses treatment protocols that provide better prophylactic or therapeutic profiles than current single agent therapies or combination therapies for inflammatory diseases, autoimmune diseases, disorders characterized by abnormal bone metabolism, disorders characterized by aberrant expression and/or activity of integrin α_{V}β₃, disorders characterized by abnormal angiogenesis, or cancer. The invention provides integrin αᵥβ₃ antagonist combination therapies for the prevention, treatment, management, or amelioration of inflammatory diseases, autoimmune diseases, disorders characterized by abnormal bone metabolism, disorders characterized by aberrant expression and/or activity of integrin α_{V}β₃, disorders characterized by abnormal angiogenesis, or cancer, or one or more conditions or symptoms associated therewith. In particular, the invention provides prophylactic and therapeutic protocols for the prevention, treatment, management or amelioration of an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer, or one or more conditions associated therewith, said protocols comprising administering to a subject in need thereof a prophylactically or therapeutically effective amount of one or more integrin αᵥβ₃ antagonists (preferably integrin αᵥβ₃ antibody, more preferably VITAXIN® or an antigen-binding fragment thereof, or an antibody or fragment thereof that competes with VITAXIN® for binding to integrin αᵥβ₃) in combination with a prophylactically or therapeutically effective amount of one or more bisphosphonates and/or one or more HMG-CoA reductase inhibitors. The invention provides prophylactic and therapeutic protocols for the prevention, treatment, management or amelioration of an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or one or more conditions associated therewith, said protocols comprising administering to a subject in need thereof a prophylactically or therapeutically effective amount of one or more integrin αᵥβ₃ antagonists (preferably integrin αᵥβ₃ antibody, more preferably VITAXIN® or an antigen-binding fragment thereof, or an antibody or fragment thereof that competes with VITAXIN® for binding to integrin αᵥβ₃) in combination with a prophylactically or therapeutically effective amount of one or more bisphosphonates and/or a prophylactically or therapeutically effective amount of one or more HMG-CoA reductase inhibitors and a prophylactically or therapeutically effective amount of one or more therapies (e.g., prophylactic or therapeutic agents) other than an integrin αᵥβ₃ antagonist, an HMG-CoA reductase inhibitor, and/or a bisphosphonate.

The invention also provides prophylactic and therapeutic protocols for the prevention, management, treatment or amelioration of an inflammatory disease, an autoimmune disorder, a disorder associated with aberrant expression and/or activity of integrin αᵥβ₃, a disorder associated with abnormal bone metabolism, a disorder associated with aberrant angiogenesis or cancer or one or more symptoms thereof, said protocols comprising administering to a subject in need thereof a prophylactically or therapeutically effective amount of one or more antibodies that immunospecifically bind to integrin αᵥβ₃ (preferably VITAXIN®) conjugated or fused to an HMG-CoA reductase inhibitor and/or a bisphosphonate. The invention also provides prophylactic and therapeutic protocols for the prevention, management, treatment or amelioration of an inflammatory disease, an autoimmune disorder, a disorder associated with aberrant expression and/or activity of integrin αᵥβ₃, a disorder associated with abnormal bone metabolism, a disorder associated with aberrant angiogenesis or cancer or one or more symptoms thereof, said protocols comprising administering to a subject in need thereof a prophylactically or therapeutically effective amount of one or more antibodies that immunospecifically bind to integrin αᵥβ₃ (preferably VITAXIN®) conjugated or fused to an HMG-CoA reductase inhibitor and/or a bisphosphonate, and a prophylactically or therapeutically effective amount of one or more therapies other than anti-integrin αᵥβ₃, antibodies, HMG-CoA reductase inhibitors, and/or bisphosphonates. In a preferred embodiment, at least one of the antibodies that immunospecifically binds to integrin αᵥβ₃ is VITAXIN® or an antigen-binding fragment thereof, or an antibody that competes with VITAXIN® or a fragment thereof for binding to integrin αᵥβ_{3.}

The present invention provides a method for preventing, managing, treating or ameliorating an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or one or more symptoms thereof, said method comprising administering to a subject in need thereof a dosage of a prophylactically or therapeutically effective amount of one or more antagonists of integrin αᵥβ₃ in combination with the administration of a dosage of a prophylactically or therapeutically effective amount of one or more HMG-CoA reductase inhibitors. In another embodiment, the invention also provides a method for preventing, managing, treating or ameliorating an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or one or more symptoms thereof, said method comprising administering to a subject in need thereof a dosage of a prophylactically or therapeutically effective amount of one or more integrin αᵥβ₃ antagonists in combination with the administration of a dosage of a prophylactically or therapeutically effective amount of one or more bisphosphonates. In another embodiment, the invention provides a method for preventing, managing, treating or ameliorating an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or one or more symptoms thereof, said method comprising administering to a subject in need thereof a dosage of a prophylactically or therapeutically effective amount of one or more antagonists of integrin αᵥβ₃ in combination with the administration of a dosage of a prophylactically or therapeutically effective amount of one or more HMG-CoA reductase inhibitors and a dosage of a prophylactically or therapeutically effective amount of one or more bisphosphonates.

In another embodiment, the invention provides a method for preventing, managing, treating or ameliorating an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or one or more symptoms thereof, said method comprising administering to a subject in need thereof a dosage of a prophylactically or therapeutically effective amount of one or more antagonists of integrin αᵥβ₃ in combination with the administration of a dosage of a prophylactically or therapeutically effective amount of one or more HMG-CoA reductase inhibitors and/or a dosage of a prophylactically or therapeutically effective amount of one or more bisphosphonates, and optionally another therapy (e.g., a therapeutic or prophylactic agent) which does not comprise an integrin α_{V}β₃ antagonist, an HMG-CoA reductase inhibitor and/or a bisphosphonate. In another embodiment, the invention provides a method for preventing, managing, treating or ameliorating an inflammatory disease, an autoimmune disorder, a disorder associated with aberrant expression and/or activity of integrin αᵥβ₃ a disorder associated with abnormal bone metabolism, a disorder associated with aberrant angiogenesis or cancer, or one or more symptoms thereof, said method comprising administering to a subject in need thereof a dosage of a prophylactically or therapeutically effective amount of one or more antagonists of integrin αᵥβ₃ conjugated or fused to an HMG-CoA reductase inhibitor and/or a bisphosphonate, and optionally another therapy (e.g., a therapeutic or prophylactic agent) which does not comprise an integrin α_{V}β₃ antagonist, an HMG-CoA reductase inhibitor or a bisphosphonate.

In accordance with the invention, a prophylactic or therapeutic agent administered in combination with an integrin αvβ₃ and an HMG-CoA reductase inhibitor and/or a bisphosphonate may or may not work by the same mechanism as the integrin αvβ₃ antagonist, HMG-CoA reductase inhibitor or bisphosphonate. Examples of integrin αvβ₃ antagonists that can be used in accordance with the invention are described in Section 4.1 infra.

In a preferred embodiment, the integrin αᵥβ₃ antagonist used in accordance with the invention is an antibody that immunospecifically binds integrin αᵥβ₃. In another preferred embodiment, the integrin αᵥβ₃ antagonist used in accordance with the invention is VITAXIN® or an antigen-binding fragment thereof or an antibody.

Examples of HMG-CoA reductase inhibitors that can be used in accordance with the invention include but are not limited to statin, cerivastatin, lescol, lupitor, rosuvastatin, atorvastatin (LIPITOR™), fluvastatin (LESCOL™), lovastatin (MEVACOR™), pravastatin (PRAVACHOL™), and simvastatin (ZOCOR™), and mixtures thereof. Examples of bisphosphonates that can be used in accordance with the invention include but are not limited to alendronate, cimadronate, clodronate, tiludronate, etidronate, ibandronate, neridronate, olpandronate, risedronate, piridronate, pamidronate, zolendronate, pharmaceutically acceptable salts thereof, and mixtures thereof.

The invention provides protocols for the administration of an integrin αᵥβ₃ antagonist in combination with an HMG-CoA reductase inhibitor and/or a bisphosphonate, and optionally in combination with another therapy (e.g., a therapeutic or prophylactic agent) which does not comprise an integrin αᵥβ₃ antagonist, an HMG-CoA reductase inhibitor, or a bisphosphonate. The individual components (e.g., individual prophylactic or therapeutic agents) of the combination therapies of the invention can be administered concomitantly or sequentially to a subject. The individual components (e.g., individual prophylactic or therapeutic agents) of the of the combination therapies of the invention can also be cyclically administered. Cycling therapy involves the administration of a first therapy (e.g., a first therapeutic or prophylactic agent) for a period of time, followed by the administration of a second therapy (e.g., a second therapeutic or prophylactic agent) for a period of time and repeating this sequential administration, i.e., the cycle, in order to reduce the development of resistance to one of the therapies, to avoid or reduce the side effects of one of the therapies and/or improve the efficacy of the therapies. The individual components (e.g., individual prophylactic or therapeutic agents) of the combination therapies of the invention can also be administered to a subject concurrently.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

The invention encompasses pharmaceutical compositions comprising one or more integrin αᵥβ₃ antagonists (preferably an antibody that immunospecifically binds integrin αᵥβ₃, more preferably VITAXIN® or an antigen-binding fragment thereof or an antibody or a fragment thereof that competes with VITAXIN® or an antigen-binding fragment thereof for binding to integrin αᵥβ₃). The invention also encompasses pharmaceutical compositions comprising one or more integrin αᵥβ₃ antagonists (preferably an antibody that immunospecifically binds integrin αᵥβ₃, more preferably VITAXIN® or an antigen-binding fragment thereof or an antibody fragment thereof that competes with VITAXIN® or an antigen-binding fragment thereof for binding to integrin αᵥβ₃), and one or more HMG-CoA reductase inhibitors. The invention also encompasses pharmaceutical compositions comprising one or more integrin αᵥβ₃ antagonists (preferably an antibody that immunospecifically binds integrin αᵥβ₃, more preferably VITAXIN® or an antigen-binding fragment thereof, or an antibody or a fragment thereof that competes with VITAXIN® or an antigen-binding fragment thereof for binding to integrin αᵥβ₃), and one or more bisphosphonates.

The invention also encompasses pharmaceutical compositions comprising one or more integrin αᵥβ₃ antagonists (preferably an antibody that immunospecifically binds an integrin αᵥβ₃, more preferably VITAXIN® or an antigen-binding fragment thereof, or an antibody or fragment thereof that competes with VITAXIN® or an antigen-binding fragment thereof for binding to integrin αᵥβ₃), one or more HMG-CoA reductase inhibitors, and one or more bisphosphonates. The invention also encompasses pharmaceutical compositions comprising one or more integrin αᵥβ₃ antagonists (preferably an antibody that immunospecifically binds an integrin αᵥβ₃, more preferably VITAXIN® or an antigen-binding fragment thereof, or an antibody or fragment thereof that competes with VITAXIN® or an antigen-binding fragment thereof for binding to integrin αᵥβ₃), one or more HMG-CoA reductase inhibitors, and one or more therapies (e.g., another prophylactic or therapeutic agent) other than an integrin αᵥβ₃ antagonist, an HMG-CoA reductase inhibitor, and/or a bisphosphonate.

The invention also encompasses pharmaceutical compositions comprising one or more integrin αᵥβ₃ antagonists (preferably an antibody that immunospecifically binds an integrin αᵥβ₃, more preferably VITAXIN® or an antigen-binding fragment thereof, or an antibody or fragment thereof that competes with VITAXIN® or an antigen-binding fragment thereof for binding to integrin αᵥβ₃), one or more HMG-CoA reductase inhibitors, one or more bisphosphonates, and one or more therapies (e.g., a prophylactic or therapeutic agent) other than an integrin αᵥβ₃ antagonist, an HMG-CoA reductase inhibitor, and/or a bisphosphonate. The invention also encompasses pharmaceutical compositions comprising one or more integrin αᵥβ₃ antagonists (preferably an antibody that immunospecifically binds integrin αᵥβ₃, more preferably VITAXIN® or an antigen-binding fragment thereof or an antibody fragment thereof that competes with VITAXIN® or a fragment thereof for binding to integrin αᵥβ₃), conjugated or fused to an HMG-CoA reductase inhibitor, and/or a bisphosphonates, and optionally one or more therapies (e.g., a prophylactic or therapeutic agent) other than an integrin αᵥβ₃ antagonist, an HMG-CoA reductase inhibitor, or a bisphosphonate.

The methods and compositions of the invention are particularly useful for the prevention, management or treatment of inflammatory disorders, autoimmune disorders, disorders associated with aberrant angiogenesis and cancer that are associated with or characterized by none-related problems such as, e.g., bone loss or bone resorption. Examples of inflammatory disorders associated with or characterized by bone-related problems include but are not limited to osteoporosis. Examples of autoimmune disorders characterized by or associated with bone-related problems include, but are not limited to, rheumatoid arthritis. Examples of disorders associated with aberrant angiogenesis that are characterized by or associated with one-related problems, include but are not limited to, bone metastases. Examples of cancers associated with or characterized by bone-related problems include, but are not limited to prostate, breast, lung and ovarian cancer.

In preferred embodiments, a disorder characterized by abnormal bone metabolism is prevented, managed, or treated in accordance with the methods of the invention. Examples of disorders characterized by abnormal bone metabolism include but are not limited to, Gorham Stout's disease and Behcet's disease.

The methods and compositions of the invention are useful not only in patients untreated for an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or a condition associated therewith, but are also useful in the prevention, treatment and management of patients partially or completely refractory to current standard and experimental therapies for such disease or disorder. Examples of such therapies include but are not limited to analgesics, anti-inflammatory agents, immunomodulatory agents, anti-angiogenic agents, bone metabolism regulating agents, chemotherapies, hormonal therapies, radiation therapies, and/or surgery. In a preferred embodiment, the methods and compositions of the invention are useful for the prevention, management, treatment or amelioration of an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, that has been shown to be or may be refractory or non-responsive to therapies other than those comprising the administration of integrin α_{V}β₃ antagonists, bisphosphonates, or HMG-CoA reductase inhibitors. The methods and compositions of the invention are also useful for the prevention, management, treatment or amelioration of an inflammatory disease, an autoimmune disorder, a disorder associated with aberrant expression and/or activity of integrin αᵥβ₃, a disorder associated with abnormal bone metabolism, a disorder associated with aberrant angiogenesis or cancer in patients that do not tolerate therapies other than antagonists for integrin α_{V}β_{3,} bisphosphonates, or HMG-CoA reductase inhibitors, because of unwanted or adverse side effects.

The invention provides articles of manufacture comprising one or more integrin αvβ3 antagonists (preferably an antibody which immunospecifically binds to integrin αvβ3, more preferably VITAXIN® or an antigen-binding fragment thereof, or an antibody or a fragment thereof that competes with VITAXIN® or an antigen-binding fragment thereof for binding to integrin αvβ3) in a first vial and one or more bisphosphonates in a second vial for use in the prevention, treatment, management or amelioration of an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or a symptom associated therewith. The invention also provides articles of manufacture comprising one or more integrin αvβ3 antagonists (preferably an antibody which immunospecifically binds to integrin αvβ3, more preferably VITAXIN® or an antibody or a fragment thereof that competes with VITAXIN® or an antigen-binding fragment thereof for binding to integrin αvβ3) in a first vial and one or more HMG-CoA reductase inhibitors in a second vial for use in the prevention, treatment, management or amelioration of an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or a symptom associated therewith. The invention also provides kits comprising one or more integrin αvβ3 antagonists (preferably an antibody which immunospecifically binds to integrin αvβ3, more preferably VITAXIN® or an antibody or a fragment thereof that competes with VITAXIN® or a fragment thereof for binding to integrin αvβ3) in a first vial, one or more bisphosphonates in a second vial, and/or one or more HMG-CoA reductase inhibitors in another vial, and optionally one or more other therapeutic agents other than integrin αvβ3 antagonists, HMG-CoA reductase inhibitors, or bisphosphonates in other vials for use in the prevention, treatment, management or amelioration of an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or symptom associated therewith. The articles of manufacture may further comprise packaging materials and/or instructions for administering such agents to a patient with an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or symptom associated therewith.

3.1 Terminology

As used herein, the term “aberrant angiogenesis” is used interchangeably with “abnormal angiogenesis”. Such terms refer to any angiogenesis that is altered relative to angiogenesis in normal healthy individuals. Alterations in angiogenesis include, but are not limited to, increased angiogenesis activity in a subject, and angiogenesis at an abnormal location of the body.

As used herein, the term “aberrant bone metabolism” is used interchangeably with the term “abnormal bone metabolism”. Such terms refer to bone metabolism that is altered relative to its functioning in a normal, healthy individual. For example, aberrant bone metabolism includes abnormal resorption of bone tissues and abnormal growth of osteocytes.

As used herein, the term “aberrant bone resorption” is used interchangeably with the term “abnormal bone resorption”. Such terms refer to bone resorption that is altered relative to that in a normal, healthy individual.

As used herein, the term “aberrant expression and/or activity of integrin αᵥβ₃” is used interchangeably with the term “expression and/or activity of integrin αᵥβ₃”. Such terms refer expression and/or activity of integrin αᵥβ₃ that is altered relative to that in a normal, healthy individual.

As used herein, the term “analog” in the context of a proteinaceous agent (e.g., proteins, polypeptides, peptides, and antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score—50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possesses a similar or identical function as a first organic or inorganic molecule and is structurally similar to the first organic or inorganic molecule.

As used herein, the terms “antagonist” and “antagonists” refer to any protein, polypeptide, peptide, peptidomimetic, glycoprotein, antibody, antibody fragment, carbohydrate, nucleic acid, organic molecule, inorganic molecule, large molecule, or small molecule that blocks, inhibits, reduces or neutralizes the function, activity and/or expression of another molecule. In various embodiments, an antagonist reduces the function, activity and/or expression of another molecule by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

As used herein, the terms “anti-integrin αᵥβ₃ antibodies,” “integrin αᵥβ₃ antibodies,” and “antibodies of the invention” refer to the antibodies described in Section 4.1.1 infra.

As used herein, the terms “antibody” and “antibodies” refer to monoclonal antibodies, multispecific antibodies, human antibodies, humanized antibodies, camelised antibodies, chimeric antibodies, single domain antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, e.g., molecules that contain an antigen binding site. Inmunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

As used herein, the term “derivative” in the context of a proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e, by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, propyl, butyl, carboxyl or amine group. An organic molecule may also be esterified, alkylated and/or phosphorylated.

As used herein, the terms “disorder” and “disease” are used interchangeably to refer to a condition in a subject. In particular, the term “inflammatory disease” is used interchangeably with the term “inflammatory disorder”. The term “autoimmune disease” is used interchangeably with the term “autoimmune disorder”. The term “disorder associated with aberrant expression and/or activity of integrin αᵥβ₃” is used interchangeably with the term “disease associated with aberrant expression and/or activity of integrin αᵥβ₃”. The term “disorder associated with abnormal bone metabolism” is used interchangeably with the term “disease associated with abnormal bone metabolism”. The term “disorder associated with aberrant angiogenesis” is used interchangeably with the term “disease associated with aberrant angiogenesis”. Certain diseases may be characterized as more than one type of disease, for example, certain diseases may be both autoimmune and inflammatory diseases.

As used herein, the term “effective amount” refers to the amount of a therapy (e.g., a prophylactic or therapeutic agent) which is sufficient to reduce or ameliorate the progression, severity and/or duration of a disease or disorder (e.g., an inflammatory disease, an autoimmune disease, a disorder characterized by abnormal bone metabolism, a disorder characterized by aberrant expression of integrin αᵥβ₃, a disorder characterized by abnormal angiogenesis, or cancer), ameliorate one or more symptoms of a disease or disorder, prevent the recurrence of a disease or disorder, prevent the development or onset of a disease or disorder, or one or more symptoms thereof, or enhance the prophylactic or therapeutic effect(s) of another therapy(ies).

As used herein, the term “epitope” refers to a fragment of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic.

The term “excipient” as used herein refers to an inert substance which is commonly used as a diluent, vehicle, preservative, binder or stabilizing agent for drugs which imparts a beneficial physical property to a formulation, such as increased protein stability, increased protein solubility, and decreased viscosity. Examples of excipients include, but are not limited to, proteins (e.g., serum albumin), amino acids (e.g., aspartic acid, glutamic acid, lysine, arginine, glycine and histidine), surfactants (e.g., SDS, polysorbate and nonionic surfactant), saccharides (e.g., glucose, sucrose, maltose and trehalose), polyols (e.g., mannitol and sorbitol), fatty acids and phospholipids (e.g., alkyl sulfonates and caprylate). For additional information regarding excipients, see Remington's Pharmaceutical Sciences (by Joseph P. Remington, 18^{th} ed., Mack Publishing Co., Easton, Pa.), which is incorporated herein in its entirety.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide or protein (including an antibody). In a specific embodiment, a fragment of a polypeptide or protein retains at least one function of the polypeptide or protein. In another embodiment, a fragment of a polypeptide or protein retains two, three or more functions of the second, different polypeptide or protein. Preferably, a fragment of an integrin αᵥβ₃ antibody retains the ability to immunospecifically bind to integrin αᵥβ₃.

As used herein, the term “fusion protein” refers to a polypeptide or protein that comprises an amino acid sequence of a first protein or polypeptide or functional fragment, analog or derivative thereof, and an amino acid sequence of a heterologous protein or polypeptide, or fragment, analog, or derivative thereof (e.g., a second protein, polypeptide, or fragment, analog or derivative thereof different than the first protein, or polypeptide, or fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. In a preferred embodiment, fusion proteins retain or have improved activity relative to the activity of the original protein, polypeptide or peptide prior to being fused to a heterologous protein, polypeptide or peptide.

As used herein, the term “host cell” includes a subject cell transfected or transformed with a nucleic acid molecule and the progeny or potential progeny of such a cell. Progeny of such a cell may not be identical to the parent cell transfected or transformed with the nucleic acid molecule due to mutations or environmental influences that may occur in succeeding generations or integration of the nucleic acid molecule into the host cell genome.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non-limiting example stringent hybridization conditions are hybridization at 6× sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (e.g., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “immunomodulatory agent” and variations thereof including, but not limited to, immunomodulatory agents, immunomodulants or immunomodulatory drugs, refer to an agent that modulates a host's immune system. In a specific embodiment, an immunomodulatory agent is an agent that shifts one aspect of a subject's immune response. In certain embodiments, an immunomodulatory agent is an agent that inhibits or reduces a subject's immune system (i.e., an immunosuppressant agent). In certain other embodiments, an immunomodulatory agent is an agent that activates or increases a subject's immune system (i.e., an immunostimulatory agent). In accordance with the invention, an immunomodulatory agent used in the combination therapies of the invention does not include an antibody that immunospecifically binds to integrin αᵥβ₃. Immunomodulatory agents include, but are not limited to, small molecules, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules.

As used herein, the term “immunospecifically binds to an antigen” and analogous terms refer to peptides, polypeptides, proteins, fusion proteins and antibodies or fragments thereof that specifically bind to an antigen or a fragment and do not specifically bind to other antigens. A peptide, polypeptide, protein, fusion protein or antibody that immunospecifically binds to an antigen may bind to other peptides, polypeptides, proteins, fusion proteins or antibodies with lower affinity as determined by, e.g., immunoassays, BIAcore, or other assays known in the art. Antibodies or fragments that immunospecifically bind to an antigen may be cross-reactive with related antigens. Preferably, antibodies or fragments that immunospecifically bind to an antigen do not cross-react with other antigens.

As used herein, the term “immunospecifically binds to integrin αᵥβ₃” and analogous terms refer to peptides, polypeptides, proteins, fusion proteins and antibodies or fragments thereof that specifically bind to integrin αᵥβ₃ or a fragment thereof and do not specifically bind to other polypeptides. A peptide, polypeptide, protein, fusion protein, antibody, or fragment thereof that immunospecifically binds to integrin αᵥβ₃ may bind to other peptides, polypeptides, proteins, fusion proteins, antibodies, or fragment thereof with lower affinity as determined by, e.g., immunoassays, BIAcore, or other assays known in the art. Antibodies or fragments that immunospecifically bind to integrin αᵥβ₃ may be cross-reactive with related antigens. Preferably, antibodies or fragments that immunospecifically bind to integrin αᵥβ₃ or fragment thereof do not cross-react with other antigens. Antibodies or fragments that immunospecifically bind to integrin αᵥβ₃ can be identified, for example, by immunoassays, BIAcore, or other techniques known to those of skill in the art. An antibody or fragment thereof binds specifically to integrin αᵥβ₃ when it binds to integrin αᵥβ₃ with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

As used herein, the term “in combination” refers to the use of more than one therapy (e.g., more than one prophylactic or therapeutic agents or treatments). The use of the term “in combination” does not restrict the order in which therapies (e.g., prophylactic and/or therapeutic agents) are administered to a subject with a disease or disorder (e.g., an inflammatory disease, an autoimmune disease, a disorder characterized by abnormal bone metabolism, a disorder characterized by aberrant expression of integrin αᵥβ₃, a disorder characterized by abnormal angiogenesis, or cancer). A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 6 months before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 6 months after) the administration of a second, third, fourth or fifth therapy to a subject with a disease or disorder (e.g., an inflammatory disease, an autoimmune disease, a disorder characterized by abnormal bone metabolism, a disorder characterized by aberrant expression and/or activity of integrin αᵥβ₃, a disorder characterized by abnormal angiogenesis, or cancer).

As used herein, the term “integrin αᵥβ₃” refers to the heterodimer integrin αᵥβ₃, an analog, derivative or a fragment thereof, or a fusion protein comprising integrin αᵥβ₃, an analog, derivative or a fragment thereof. The integrin αᵥβ₃ may be from any species. The nucleotide and/or amino acid sequences of integrin αᵥβ₃ can be found in the literature or public databases, or the nucleotide and/or amino acid sequences can be determined using cloning and sequencing techniques known to one of skill in the art. For example, the nucleotide sequence of human integrin αᵥβ₃ can be found in the GenBank database (see, e.g., Accession No. NM_{—}002210 for α_{V}, and Accession No. L28832 for β₃). The amino acid sequence of human α_{V}β₃ can be found in the GenBank database (see, e.g., Accession No. AAA 61631 for α_{V}, and Accession No. S44360 for β₃). In a preferred embodiment, an integrin α_{V}β₃ is human integrin α_{V}β₃, an analog, derivative or a fragment thereof.

As used herein, the terms “antagonist of integrin αᵥβ₃” and “integrin αᵥβ₃ antagonist” are used interchangeably to refer to the antagonists described in section 4.1 infra.

As used herein, the term “isolated” in the context of a compound refers to a compound which is substantially free of a different, contaminating compound.

As used herein, the term “isolated” in the context of a proteinaceous agent (e.g., peptide, polypeptide, protein, fusion protein or antibody) refers to a proteinaceous agent which is substantially free of cellular material or contaminating proteinaceous agents from the cell or tissue source from which it is derived, or substantially free of chemical precursors or other chemicals when chemically synthesized. The language “substantially free of cellular material” includes preparations of proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous proteinaceous agents (also referred to as a “contaminating proteinaceous agent”). When the proteinaceous agent is recombinantly produced, it is also preferably substantially free of culture medium, e.g., culture medium represents less than about 20%, 10%, or 5% of the volume of the proteinaceous agent preparation. When the proteinaceous agent is produced by chemical synthesis, it is preferably substantially free of chemical precursors or other chemicals, e.g., it is separated from chemical precursors or other chemicals which are involved in the synthesis of the proteinaceous agent. Accordingly such preparations of a proteinaceous agent have less than about 30%, 20%, 10%, 5% (by dry weight) of chemical precursors or compounds other than the proteinaceous agent of interest. In a preferred embodiment, an antibody of the invention is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment, a nucleic acid molecule encoding an antibody of the invention is isolated.

As used herein, the terms “manage”, “managing” and “management” refer to the beneficial effects that a subject derives from a therapy (e.g., prophylactic or therapeutic agent), which does not result in a cure of the disease. In certain embodiments, a subject is administered two or more therapies (e.g., two or more prophylactic or therapeutic agents) to “manage” a disease so as to prevent the progression or worsening of the disease.

As used herein, the terms “non-responsive” and refractory“describe patients treated with a currently available therapy (e.g., prophylactic or therapeutic agent) for a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) which is not clinically adequate to relieve one or more symptoms associated with the disease or disorder (e.g., an inflammatory disease, an autoimmune disease, a disorder characterized by abnormal bone metabolism, a disorder characterized by aberrant expression of integrin α_{V}β₃, a disorder characterized by abnormal angiogenesis, or cancer). Typically, such patients suffer from severe, persistently active disease and require additional therapy to ameliorate the symptoms associated with their disease or disorder.

As used herein, the terms “prophylactic agent” and “prophylactic agents” refer to any agent(s) which can be used in the prevention of a disease or disorder (e.g., an inflammatory disease, an autoimmune disease, a disorder characterized by abnormal bone metabolism, a disorder characterized by aberrant expression of integrin α_{V}β₃, a disorder characterized by abnormal angiogenesis, or cancer) or one or more symptoms thereof. In certain embodiments, the term “prophylactic agent” refers to an integrin αᵥβ₃ antagonist (e.g., an anti-integrin α_{V}β₃ antibody). In certain other embodiments, the term “prophylactic agent” does not refer to an integrin αᵥβ₃ antagonist. In other embodiments, the term “prophylactic agent” refers to an HMG-CoA reductase inhibitor or a bisphosphonate. In other embodiments, the term “prophylactic agents” refers to an integrin αᵥβ₃ antagonist and a bisphosphonate. In other embodiments, the term “prophylactic agents” refers to an integrin αvβ3 antagonist and an HMG-CoA reductase inhibitor. In yet other embodiments, the term “prophylactic agents” refers to an integrin αᵥβ₃ antagonist, a bisphosphonate, and an HMG-CoA reductase inhibitor. In yet another embodiment, the term “prophylactic agents” refers to an integrin αvβ3 antagonist, a bisphosphonate and/or an HMG-CoA reductase inhibitor, and a prophylactic agent other than an integrin αᵥβ₃ antagonist, a bisphophonate or an HMG-CoA reductase inhibitor. Preferably, a prophylactic agent is an agent which is known to be useful to, or has been or is currently being used to the prevent or impede the onset, development, progression and/or severity of a disease or disorder (e.g., an inflammatory disease, an autoimmune disease, a disorder characterized by abnormal bone metabolism, a disorder characterized by aberrant expression of integrin α_{V}β₃, a disorder characterized by abnormal angiogenesis, or cancer) or one or more symptoms thereof. Prophylactic agents may be characterized as different agents based upon one or more effects that the agents have in vitro and or in vivo.

As used herein, the terms “prevent”, “preventing” and “prevention” refer to the prevention of the development, recurrence or onset of a disease or disorder (e.g., an inflammatory disease, an autoimmune disease, a disorder characterized by abnormal bone metabolism, a disorder characterized by aberrant expression of integrin α_{V}β₃, a disorder characterized by abnormal angiogenesis, or cancer) or one or more symptoms thereof in a subject resulting from the administration of a therapy (e.g., prophylactic agent), or the administration of a combination of therapies (e.g., a combination of prophylactic or therapeutic agents).

As used herein, the term “prophylactically effective amount” refers to that amount of the therapy (e.g., a prophylactic agent) sufficient to result in the prevention of the development, recurrence or onset of a disease or disorder (e.g., an inflammatory disease, an autoimmune disease, a disorder characterized by abnormal bone metabolism, a disorder characterized by aberrant expression of integrin α_{V}β₃, a disorder characterized by abnormal angiogenesis, or cancer) or one or more symptoms thereof, or to enhance or improve the prophylactic effects of another therapy (e.g., another prophylactic or therapeutic agent). Examples of suitable dosages of prophylactically effective amounts of agents are given in Sections 4.5.1 through 4.5.13 infra.

As used herein, a “prophylactic protocol” refers to a regimen for dosing and timing the administration of one or more prophylactic therapies (e.g., one or more prophylactic agents).

A used herein, a “protocol” includes dosing schedules and dosing regimens. The protocols herein are methods of use and include prophylactic and therapeutic protocols.

As used herein, the phrase “side effects” encompasses unwanted and adverse effects of a therapy (e.g., one or more prophylactic or therapeutic agents). Side effects are always unwanted, but unwanted effects are not necessarily adverse. An adverse effect from therapy (e.g., a prophylactic or therapeutic agent) might be harmful or uncomfortable or risky. Side effects typically experienced by patients administered a therapy are numerous and known in the art. Many are described in the Physicians' Desk Reference (57^{th} ed., 2003).

Side effects from administration of REMICADE™ include, but are not limited to, risk of serious infection and hypersensitivity reactions. Other side effects range from nonspecific symptoms such as fever or chills, pruritus or urticaria, and cardiopulmonary reactions such as chest pain, hypotension, hypertension or dyspnea, to effects such as myalgia and/or arthralgia, rash, facial, hand or lip edema, dysphagia, sore throat, and headache. Yet other side effects include, but are not limited to, abdominal hernia, splenic infarction, splenomegaly, dizziness, upper motor neuron lesions, lupus erythematosus syndrome, rheumatoid nodules, ceruminosis, abdominal pain, diarrhea, gastric ulcers, intestinal obstruction, intestinal perforation, intestinal stenosis, nausea, pancreatitis, vomiting, back pain, bone fracture, tendon disorder or injury, cardiac failure, myocardial ischema, lymphoma, thrombocytopenia, cellulitis, anxiety, confusion, delirium, depression, somnolence, suicide attempts, anemia, abscess, bacterial infections, and sepsis.

Side effects from administration of ENBREL™ include, but are not limited to, risk of serious infection and sepsis, including fatalities. Adverse side effects range from serious infections such as pyelonephritis, bronchitis, septic arthritis, abdominal abscess, cellulitis, osteomyclitis, wound infection, pneumonia, foot abscess, leg ulcer, diarrhea, sinusitis, sepsis, headache, nausea, rhinitis, dizziness, pharyngitis, cough, asthenia, abdominal pain, rash, peripheral edema, respirator disorder, dyspepsia, sinusitis, vomiting, mouth ulcer, alopecia, and pheumonitis to other less frequent adverse effects such as heart failure, myocardial infarction, myocardia ischemia, cerebral ischemia, hyertension, hypotension, cholcystitis, pancreatitis, gastrointestinal hemorrhage, bursitis, depression, dyspnea, deep vein thrombosis, pulmonary embolism, membranous glomerulonephropathy, polymyositis, and thrombophlebitis. The side effects resulting from administration of methotrexate include, but are not limited to, serious toxic reactions, which can be fatal, such as unexpectedly severe bone marrow suppression, gastrointestinal toxicity, hepatotoxicity, fibrosis and cirrhosis after prolonged use, lung diseases, diarrhea and ulcerative stomatitis, malignant lymphomas and occasionally fatal severe skin reactions.

Side effects from chemotherapy include, but are not limited to, gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure, as well as constipation, nerve and muscle effects, temporary or permanent damage to kidneys and bladder, flu-like symptoms, fluid retention, and temporary or permanent infertility.

Side effects from radiation therapy include but are not limited to fatigue, dry mouth, and loss of appetite. Other side effects include gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure.

Side effects from biological therapies/immunotherapies include but are not limited to rashes or swellings at the site of administration, flu-like symptoms such as fever, chills and fatigue, digestive tract problems and allergic reactions.

Side effects from hormonal therapies include but are not limited to nausea, fertility problems, depression, loss of appetite, eye problems, headache, and weight fluctuation.

Side effects from bisphosphonates include but are not limited to drop in calcium levels, change in kidney function, increased pain, bloating, gas, heartburn, nausea, headache, high temperature and chills, dizziness, blurred vision, occular irritation, non-specific conjunctivitis (pain, epiphoria, photophobia), anterior uveitis, anterior scleritis, episcleritis, periocular, lid and/or orbital edema, liver damage, muscle aches, tenderness and weakness.

Side effects from HMG-CoA reductase inhibitors include but are not limited to liver damage, stomach upset or pain, diarrhea, constipation, headache, dizziness, skin rash, muscle tenderness or soreness, unexplained muscle pain, general malaise, fatigue and weakness, and fever.

As used herein, the term “small molecules” and analogous terms include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a chimpanzee, a monkey such as a cynomolgous monkey, and a human), and more preferably a human. In one embodiment, the subject is a mammal, preferably a human, with an inflammatory disease. In one embodiment, the subject is a mammal, preferably a human, with an autoimmune disease. In one embodiment, the subject is a mammal, preferably a human, with a disorder characterized by abnormal bone metabolism, or a condition associated therewith. In one embodiment, the subject is a mammal, preferably a human, with a disorder characterized by aberrant expression of integrin α_{V}β₃, or a condition associated therewith In one embodiment, the subject is a mammal, preferably a human, with a disorder characterized by abnormal angiogenesis. In one embodiment, the subject is a mammal, preferably a human, with cancer. In another embodiment, the subject is a mammal, preferably a human, with an increased risk of developing an inflammatory disease, an autoimmune disease, a disorder characterized by abnormal bone metabolism, a disorder characterized by aberrant expression of integrin α_{V}β₃, a disorder characterized by abnormal angiogenesis, or cancer, or one or more symptoms associated therewith. In another embodiment, the subject is a mammal, preferably a human, refractory or non-responsive to available single or combination therapies. In yet another embodiment, the subject is a farm animal (e.g., a cow, pig, or horse) or a pet (e.g., a cat or dog).

As used herein, the term “synergistic” refers to a combination of therapies (e.g., a combination of prophylactic or therapeutic agents) which is more effective than the additive effects of any two or more single agents. A synergistic effect of a combination of therapies (e.g., prophylactic or therapeutic agents) permits the use of lower dosages of one or more of the therapies (e.g., prophylactic or therapeutic agents) and/or less frequent administration of said therapies (e.g., agents) to a subject with a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) or a condition or symptom associated therewith. The ability to utilize lower dosages of therapies (e.g., prophylactic or therapeutic agents) and/or to administer said therapies (e.g., agents) less frequently reduces the toxicity associated with the administration of said therapies (e.g., agents) to a subject without reducing the efficacy of said therapies (e.g., therapeutic or prophylactic agents) in the prevention, management, or treatment of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) or a condition or symptom associated therewith. In addition, a synergistic effect can result in improved efficacy of therapies (e.g., agents) in the prevention, management, or treatment of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) or a condition or symptom associated therewith. Finally, synergistic effect of a combination of therapies (e.g., prophylactic or therapeutic agents) may avoid or reduce adverse or unwanted side effects associated with the use of any single therapy.

As used herein, the terms “therapeutic agent” and “therapeutic agents” refer to any agent(s) which can be used in the treatment, management or amelioration of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) or one or more symptoms thereof. In certain embodiments, the term “therapeutic agent” refers to an integrin αᵥβ₃ antagonist (e.g., an anti-integrin αᵥβ₃ antibody). In certain other embodiments, the term “therapeutic agent” does not refer to an integrin αᵥβ₃ antagonist (e.g., an anti-integrin αᵥβ₃ antibody). In other embodiments, the term “therapeutic agent” refers to an HMG-CoA reductase inhibitor or a bisphosphonate. In other embodiments, the term “therapeutic agents” refers to an integrin αᵥβ₃ antagonist and a bisphosphonate. In other embodiments, the term “therapeutic agents” refers to an integrin αᵥβ₃ antagonist and an HMG-CoA reductase inhibitor. In other embodiments, the term “therapeutic agents” refers to an integrin αᵥβ₃ antagonist, a bisphosphonate, and an HMG-CoA reductase inhibitor. In yet other embodiments, the term “therapeutic agents” refers to an integrin αᵥβ₃ antagonist, an HMG-CoA reductase inhibitor and/or a bisphosphonate, and one or more other therapeutic or prophylactic therapies, other than an integrin αᵥβ₃ antagonist, a bisphosphonate, and an HMG-CoA reductase inhibitor. Preferably, a therapeutic agent is an agent which is known to be useful for, or has been or is currently being used for the treatment or amelioration of a disease or disorder (e.g., an inflammatory disease, an autoimmune disease, a disorder characterized by abnormal bone metabolism, a disorder characterized by aberrant expression of integrin α_{V}β₃, a disorder characterized by abnormal angiogenesis, or cancer) or one or more symptoms thereof. Therapeutic agents may be characterized as different agents based upon one or more effects that the agents have in vitro and/or in vivo. For example, a particular anti-inflammatory agent may also be characterized as an anti-arthritic agent.

As used herein, the term “therapeutically effective amount” refers to that amount of a therapy (e.g., therapeutic agent) sufficient to result in the amelioration of one or more symptoms of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) such as pain or fatigue, prevent the advancement of a disease or disorder, cause regression of a disease or disorder, or to enhance or improve the therapeutic effect(s) of another therapy (e.g., another therapeutic agent). In certain embodiments, with respect to the treatment of an inflammatory disease, a therapeutically effective amount is an amount reduces inflammation of a joint, tissue or organ in a subject. Preferably, a therapeutic agent that reduces the amount of inflammation of a joint, tissue or organ in a subject by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a control such as PBS. In other embodiments, with respect to the treatment of a disorder associated with bone resorption, (e.g., Gorham-Stout disease and osteoporosis), a therapeutically effective amount refers to the amount of a therapeutic agent that reduces the amount of bone resorption in a subject. Preferably, a therapeutically effective amount of a therapeutic agent reduces the amount of bone resorption in a subject by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as PBS. In other embodiments, with respect to the treatment of abnormal angiogenesis, a therapeutically effective amount refers to the amount of a therapeutic agent that reduces the rate of angiogenesis in a subject. Preferably, a therapeutically effective amount of a therapeutic agent reduces the rate of angiogenesis in a subject by at least 5%, preferably at least 10%, at least 15%, at least 20%, at least 25%, at least 30%, at least 35%, at least 40%, at least 45%, at least 50%, ate least 55%, at least 60%, at least 65%, at least 70%, at least 75%, at least 80%, at least 85%, at least 90%, at least 95% or at least 99% relative to a control such as PBS. In other embodiments, with respect to the treatment of cancer, a therapeutically effective amount refers to the amount of a therapeutic agent that e.g., reduces te proliferation of cancer cells, increases the death of cancer cells or, reduces the size of a tumor or spread of a tumor in a subject. Preferably, a therapeutically effective amount of a therapeutic agent reduces the size of a tumor or the spread of a tumor in a subject by at least 5%, preferably at least 10%, at least 15%, at least 20%, at least 25%, at least 30%, at least 35%, at least 40%, at least 45%, at least 50%, ate least 55%, at least 60%, at least 65%, at least 70%, at least 75%, at least 80%, at least 85%, at least 90%, at least 95% or at least 99% relative to a control such as PBS.

As used herein, the term “therapeutic protocol” refers to a regimen for dosing and timing the administration of one or more therapies (e.g., one or more prophylactic or therapeutic agents).

As used herein, the terms “therapies” and “therapy” can refer to any protocol(s), method(s) and/or agent(s) that can be used in the prevention, treatment, management or amelioration of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) or one or more symptoms thereof known to a medical personnel (e.g., a doctor or nurse) or researcher skilled in the art.

The terms “treat”, “treatment” and “treating” as used herein refer to the reduction or amelioration of the progression, severity, and/or duration of a disease or disorder (e.g., an inflammatory disease, an autoimmune disease, a disorder characterized by abnormal bone metabolism, a disorder characterized by aberrant expression and/or activity of integrin α_{V}β₃, a disorder characterized by abnormal angiogenesis, or cancer) or the amelioration of one or more symptoms associated with said disease or disorder resulting from the administration of one or more therapies (including but not limited to, the administration of one or more prophylactic or therapeutic agents, radiation therapy, antibody, gene therapy, steroid therapy, hormone therapy, surgery, physical therapy, and any other methods or agents that can be used). In certain embodiments, such terms refer to a reduction in the swelling of one or more joints, organs or tissues, or a reduction in the pain associated with a disease or disorder (e.g., an autoimmune or inflammatory disorder). In other embodiments, such terms refer to the inhibition or reduction in the proliferation of cancerous cells, the inhibition or reduction the spread of tumor cells (metastasis), the inhibition or reduction in the onset, development or progression of one or more symptoms associated with cancer, or the reduction in the size of a tumor. In other embodiments, such terms refer to inhibition or reduction in bone loss or bone resorption. In yet other embodiments, such terms refer to inhibition or reduction in abnormal angiogenesis.

### 4. DETAILED DESCRIPTION OF THE INVENTION

The present invention encompasses treatment protocols that provide better prophylactic and therapeutic profiles than current single agent therapies or combination therapies for inflammatory diseases, autoimmune disorders, disorders associated with aberrant expression and/or activity of integrin αᵥβ₃, disorders associated with abnormal bone metabolism, disorders associated with aberrant angiogenesis, cancer, and/or one or more conditions or symptoms associated therewith. In particular, the present invention provides methods of treating, preventing, managing or ameliorating an inflammatory disease, an autoimmune disorder, a disorder associated with aberrant expression and/or activity of integrin αᵥβ₃, a disorder associated with abnormal bone metabolism, a disorder associated with aberrant angiogenesis, cancer, and/or one or more symptoms or conditions associated therewith, comprising administering to a subject in need an integrin αᵥβ₃ antagonist in combination with an HMG-CoA reductase inhibitor and/or a bisphosphonate, and optionally in combination with one or more therapies (e.g., another prophylactic or therapeutic agent) other than an integrin αᵥβ₃ antagonist, an HMG-CoA reductase inhibitor, and/or a bisphosphonate.

The present invention provides pharmaceutical compositions comprising one or more integrin αᵥβ₃ antagonists and one or more HMG-CoA reductase inhibitors, and optionally, one or more therapies other than an integrin αᵥβ₃ antagonist and an HMG-CoA reductase inhibitor. The present invention also provides pharmaceutical compositions comprising one or more integrin αᵥβ₃ antagonists and one or more bisphosphonates, and optionally, one or more therapies other than an integrin αᵥβ₃ antagonist and an HMG-CoA reductase inhibitor. The present invention also provides pharmaceutical compositions comprising one or more integrin αᵥβ₃ antagonists and one or more HMG-CoA reductase inhibitors, one or more bisphosphonates, and optionally, one or more therapies other than an integrin αᵥβ₃ antagonist, and/or an HMG-CoA reductase inhibitor. Further, the invention encompasses articles of manufacture comprising said pharmaceutical compositions and the use of said compositions in the prevention, treatment, management or amelioration of an inflammatory disease, an autoimmune disorder, a disorder associated with aberrant expression and/or activity of integrin αᵥβ₃, a disorder associated with abnormal bone metabolism, a disorder associated with aberrant angiogenesis, cancer, or one or more symptoms thereof.

4.1 Integrin αᵥβ₃ Antaponists

Any integrin α_{V}β₃ antagonist well-known to one of skill in the art may be used in the methods and compositions of the invention. The invention encompasses the use of one or more integrin α_{V}β₃ antagonists in the compositions and methods of the invention. Examples of integrin α_{V}β₃ antagonists include, but are not limited to, proteinaceous agents such as non-catalytic metalloproteinase fragments, RGD peptides, peptide mimetics, fusion proteins, disintegrins or derivatives or analogs thereof, and antibodies that immunospecifically bind to integrin α_{V}β₃, nucleic acid molecules, organic molecules, and inorganic molecules. Non-limiting examples of RGD peptides recognized by integrin α_{V}β₃ include Triflavin. Examples of antibodies that immunospecifically bind to integrin α_{V}β₃ include, but are not limited to, D12 (Smith Kline Beecham Corp.; International Publication No. WO 98/40488, antibodies disclosed in International Publication No. WO 98/46264, LM609 (Scripps; International Publication No. WO 89/015155 and U.S. Pat. No. 5,752,230, which is incorporated herein by reference in its entirety), and MEDI-522 (a.k.a. VITAXIN®; MedImmune, Inc., Gaithersburg, Md.; Wu et al., 1998, PNSA USA 95(11):6037-6042; International Publication Nos. WO 90/33919 and WO 00/78815, each of which is incorporated herein by reference in its entirety), 17661-37E and 17661-37E 1-5 (US Biological), MON 2032 (CalTag), ab7166 (BV3) and ab 7167 (BV4) (Abcam), and WOW-1 (Kiosses et al., Nature Cell Biology 3:316-320). Non-limiting examples of small molecule peptidometric integrin α_{V}β₃ antagonists include S836 (Searle) and S448 (Searle). Examples of disintegrins include, but are not limited to, Accutin. The invention also encompasses the use of any of the integrin α_{V}β₃ antagonists disclosed in the following U.S. patents and U.S. patent application Publications in the compositions and methods of the invention: U.S. Pat. Nos. 6,410,526; 6,426,353; 6,472,403; 6,344,484; 6,316,412; 6,297,249; 6,294,549; 6,274,620; 6,268,378; 6,232,308; 6,211,184; 6,204,282; 6,193,968; 6,171,588; 6,160,099; 6,153,628; 6,130,231; 6,127,335; 6,100,423; 6,096,707; 6,090,944; 6,066,648; 6,048,861; 6,037,176; 6,017,926; 6,017,925; 5,985,278; 5,981,546; 5,981,478; 5,955,572; 5,952,341; 5,925,655; 5,919,792; 5,877,281; 5,852,210; 5,849,865; 5,849,692; 5,830,678; 5,843,906; 5,843,774; 5,817,457; 5,807,819; 5,792,745; 5,780,426; 5,773,646; 5,773,644; 5,773,412; 5,770,565; 5,767,071; 5,766,591; 5,760,029; 5,760,028; 5,759,996; 5,753,230; 5,710,159; 5,705,481; 5,693,612; 5,681,820; 5,652,110; 5,652,109; 5,578,704; 5,589,570; 5,523,209; 5,498,694; 5,478,725; 5,306,620; 5,262,520; 5,204,445; 5,196,511; 5,190,873; 5,149,780; and U.S. patent application Publication Nos. 20020019402; 20020019387; 20020010176; 20020001840; 20010053853; 20010044535; 20010023242; 20010016645; 20010011125; and 20010001309, which are all incorporated herein by reference in their entireties.

In certain embodiments, an integrin αᵥβ₃ antagonist is a small organic molecule. In other embodiments, an integrin αᵥβ₃ antagonist is not a small organic molecule. In particular embodiments, an integrin αᵥβ₃ antagonist does not include any of the compounds disclosed in U.S. Pat. Nos. 6,410,526; 6,426,353; and 6,472,403. In a preferred embodiment, an integrin αᵥβ₃ antagonist is an antibody that immunospecifically binds to integrin αᵥβ₃. In another preferred embodiment, an integrin αᵥβ₃ antagonist is VITAXIN®, a derivative, analog, or antigen-binding fragment thereof, or an antibody that competes with VITAXIN® for binding to integrin αvβ3. In other embodiments, an integrin αᵥβ₃ antagonist is an antibody other than VITAXIN® or an antigen-binding fragment thereof, that imunospecifically binds to integrin αᵥβ₃. In yet other embodiments, an integrin αᵥβ₃ antiagonist is an antibody other than D12 that competes with VITAXIN® for binding to intergin αᵥβ₃.

In a preferred embodiment, an integrin αᵥβ₃ antagonist inhibits or reduces bone resorption. In another preferred embodiment, an integrin αᵥβ₃ antagonist inhibits or reduces angiogenesis. In a preferred embodiment, proteins, polypeptides or peptides (including antibodies and fusion proteins) that are utilized as integrin α_{V}β₃ antagonists are derived from the same species as the recipient of the proteins, polypeptides or peptides so as to reduce the likelihood of an immune response to those proteins, polypeptides or peptides. In another preferred embodiment, when the subject is a human, the antibodies that are utilized as integrin α_{V}β₃ antagonists are human or humanized.

Nucleic acid molecules encoding proteins, polypeptides, or peptides (including antibodies and fusion proteins) that function as integrin α_{V}β₃ antagonists, or proteins, polypeptides, or peptides that function as integrin α_{V}β₃ antagonists can be administered to a subject with an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or symptom associated therewith. Examples of such disorders include but are not limited to osteoporosis, osteopenia, osteodystrophy, arthritis, Paget's disease, myositis ossificans, Bechterew's disease, metatstatic bone disease, periodontal disease, cholelithiasis, nephrolithiasis, urolithiasis, urinary calculus, hardening of the arteries (sclerosis), arthritis, rheumatoid arthritis, bone metastasis, bursitis, neuritis, tetany, heterotropic ossificans, hypercalcaemia of malignancy, multiple myeloma, breast cancer, prostate cancer, and hypercholesterolemia, in accordance with the methods of the invention. Further, nucleic acid molecules encoding derivatives, analogs, fragments or variants of proteins, polypeptides, or peptides that function as integrin αᵥβ₃ antagonists, or derivatives, analogs, fragments or variants of proteins, polypeptides, or peptides that function as integrin α_{V}β₃ antagonists can be administered to a subject with an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or symptom associated therewith in accordance with the methods of the invention. Preferably, such derivatives, analogs, variants and fragments retain the integrin α_{V}β₃ antagonist activity of the full-length wild-type protein, polypeptide, or peptide.

4.1.1 Antibodies that Immunospecifically Bind to Integrin αᵥβ₃

It should be recognized that antibodies that immunospecifically bind to integrin α_{V}β₃ and function as antagonists are known in the art. Examples of known antibodies that immunospecifically bind to integrin α_{V}β₃ include, but are not limited to, D12 (Smith Kline Beecham Corp., International Publication No. WO 98/40498, the antibodies disclosed in International Publication No. WO 98/46264, the murine monoclonal LM609 (Scripps; International Publication No. WO 89/015155 and U.S. Pat. No. 5,753,230, which are incorporated herein by reference in their entireties), the humanized monoclonal antibody MEDI-522 (a.k.a. VITAXIN®, MedImmune, Inc., Gaithersburg, Md.; Wu et al., 1998, PNAAS USA 95(11):6037-6042; International Publication No. WO 90/33919 and WO 00/78815; each of which is incorporated herein by reference in its entirety), 17661-37E and 17661-37E 1-5 (US Biological), MON 2032 (CalTag), ab7166 (BV3) and ab 7167 (BV4) (Abcam), and WOW-1 (Kiosses et al., Nature Cell Biology 3:316-320).

The antibodies that immunospecifically bind to integrin αᵥβ₃ may be from any animal origin including birds and mammals (e.g., human, murine, donkey, sheep, rabbit, goat, guinea pig, camel, horse, or chicken). Preferably, the antibodies that immunospecifically bind to integrin αᵥβ₃ are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies that immunospecifically bind to integrin αᵥβ₃ may be monospecific, bispecific, trispecific or of greater multispecificity. Multispecific antibodies may be specific for different epitopes of integrin αᵥβ₃ or may be specific for both an integrin αᵥβ₃ epitope as well as for a heterologous epitope, such as a heterologous polypeptide or solid support material. See, e.g., International Publication Nos. WO 93/17715, WO 92/08802, WO 91/00360, and WO 92/05793; Tutt, et al., J. Immunol. 147:60-69 (1991); U.S. Pat. Nos. 4,474,893, 4,714,681, 4,925,648, 5,573,920, and 5,601,819; and Kostelny et al., 1992, J. Immunol. 148:1547-1553.

The present invention encompasses the use of antibodies that have a high binding affinity for integrin αᵥβ₃. In a specific embodiment, an antibody that immunospecifically binds to integrin αᵥβ₃ has an association rate constant or kₒₙ rate (antibody (Ab)+antigen (Ag) [symbol] Ab−Ag) of at least 10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹. In a preferred embodiment, an antibody that immunospecifically binds to integrin αᵥβ₃ has a kₒₙ of at least 2×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹.

In another embodiment, an antibody that immunospecifically binds to integrin αᵥβ₃ has a k_{off} rate (antibody (Ab)+antigen (Ag) [symbol] Ab−Ag) of less than 10⁻¹s⁻¹, less than 5×10⁻¹s⁻¹, less than 10⁻²s⁻¹, less than 5×10⁻²s⁻¹, less than 5×10⁻³s⁻¹, less than 10⁻⁴s⁻¹, less than 5×10⁻⁴s⁻¹, less than 10⁻⁵s⁻¹s⁻¹, less than 10⁻⁶s⁻¹, less than 5×10⁻⁶s⁻¹, less than 10⁻⁷s⁻¹, less than 5×10⁻⁵s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰s⁻¹. In a preferred embodiment, an antibody that immunospecifically binds to integrin αᵥβ₃ has a kₒₙ of less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰s⁻¹.

In another embodiment, an antibody that immunospecifically binds to integrin αᵥβ₃ has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In yet another embodiment, an antibody that immunospecifically binds to integrin αᵥβ₃ has a dissociation constant or K_{d} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M.

In a specific embodiment, an antibody that immunospecifically binds to integrin αᵥβ₃ is LM609 or an antigen-binding fragment thereof (e.g., one or more complementarity determining regions (CDRs) of LM609). LM609 has the amino acid sequence disclosed, e.g., in International Publication No. WO 89/05155 (which is incorporated herein by reference in its entirety), or the amino acid sequence of the monoclonal antibody produced by the cell line deposited with the American Type Culture Collection (ATCC™), 10801 University Boulevard, Manassas, Va. 20110-2209 as Accession Number HB 9537. In an alternative embodiment, an antibody that immunospecifically binds to integrin αᵥβ₃ is not LM609 or an antigen-binding fragment of LM609.

In a particular embodiment, an antibody that immunospecifically binds to αᵥβ₃ competes with VITAXIN® or an antigen-binding fragment thereof for binding to integrin αᵥβ₃. In another embodiment, an antibody that immunospecifically binds to integrin αᵥβ₃ is an antibody other than D12 that competes with VITAXIN® or an antigen-binding fragment thereof for binding to integrin αᵥβ₃. In other embodiments, an antibody that immunospecifically binds to integrin αᵥβ₃ does not compete with VITAXIN® or an antigen-binding fragment thereof for binding antibodies or fragments thereof that compete with VITAXIN® or an antigen-binding fragment thereof for binding to integrin αᵥβ₃ can be identified using techniques well-known to one of skill in the art, including, but not limited to, competitive immunoassays.

In a preferred embodiment, an antibody that immunospecifically binds to integrin αᵥβ₃ is VITAXIN® or an antibody-binding fragment thereof (e.g., one or more CDRs of VITAXIN®). VITAXIN® is disclosed, e.g., in International Publication No. WO 98/33919 and WO 00/78815, and U.S. patent application Ser. No. 09/339,922, each of which is incorporated herein by reference in its entirety. In an alternative embodiment, an antibody that immunospecifically binds to integrin αᵥβ₃ is an antibody other than VITAXIN® or an antigen-binding fragment of VITAXIN®.

The present invention encompasses the use of antibodies that immunospecifically bind integrin αᵥβ₃, said antibodies comprising a variable heavy (“VH”) domain having an amino acid sequence of the VH domain for LM609 or VITAXIN®. The present invention also encompasses the use of antibodies that immunospecifically bind to integrin αᵥβ₃, said antibodies comprising a VH CDR or a combination of VH CDRs having an amino acid sequence of any one of the VH CDRs or a combination of the VH CDRs listed in Table 1 infra.

TABLE 1
CDR Sequences Of LM609

| CDR | Sequence | SEQ ID NO: |
|---|---|---|
| VH1 | SYDMS | 1 |
| VH2 | KVSSGGG | 2 |
| VH3 | HNYGSFAY | 3 |
| VL1 | QASQSISNHLH | 4 |
| VL2 | YRSQSIS | 5 |
| VL3 | QQSGSWPHT | 6 |

In one embodiment, antibodies that immunospecifically bind to integrin αᵥβ₃ comprise a VH CDR1 having the amino acid sequence of SEQ ID NO:1. In another embodiment, antibodies that immunospecifically bind to integrin αᵥβ₃ comprise a VH CDR2 having the amino acid sequence of SEQ ID NO:2. In another embodiment, antibodies that immunospecifically bind to integrin αᵥβ₃ comprise a VH CDR3 having the amino acid sequence of SEQ ID NO:3. In a preferred embodiment, antibodies that immunospecifically bind to integrin αᵥβ₃, comprise a VH CDR1 having the amino acid sequence of SEQ ID NO:1, a VH CDR2 having the amino acid sequence of SEQ ID NO:2, and a VH CDR3 having the amino acid sequence of SEQ ID NO:3.

The present invention encompasses the use of antibodies that immunospecifically bind to integrin αᵥβ₃, said antibodies comprising a variable light (“VL”) domain having an amino acid sequence of the VL domain for LM609 or VITAXIN®. The present invention encompasses the use of antibodies that immunospecifically bind to integrin αᵥβ₃ said antibodies comprising a VL CDR or a combination of VL CDRs having an amino acid sequence of any one of the VL CDRs or a combination of the VL CDRs listed in Table 1.

In one embodiment, antibodies that immunospecifically bind to integrin αᵥβ₃ comprise a VL CDR1 having the amino acid sequence of SEQ ID NO:4. In another embodiment, antibodies that immunospecifically bind to integrin αᵥβ₃ comprise a VL CDR2 having the amino acid sequence of SEQ ID NO:5. In another embodiment, antibodies that immunospecifically bind to integrin αᵥβ₃ comprise a VL CDR3 having the amino acid sequence of SEQ ID NO:6. In a preferred embodiment, antibodies that immunospecifically bind to integrin αᵥβ₃ comprise a VL CDR1 having the amino acid sequence of SEQ ID NO:4, a VL CDR2 having the amino acid sequence of SEQ ID NO:5, and a VL CDR3 having the amino acid sequence of SEQ ID NO:6.

The present invention encompasses the use of antibodies that immunospecifically bind to integrin αᵥβ₃, said antibodies comprising a VH domain disclosed herein combined with a VL domain disclosed herein, or other VL domain. The present invention encompasses the use of antibodies that immunospecifically bind to integrin αᵥβ₃, said antibodies comprising a VL domain disclosed herein combined with a VH domain disclosed herein, or other VH domain.

The present invention encompasses the use of antibodies that immunospecifically bind to integrin αᵥβ₃, said antibodies comprising one, two or more VH CDRs and one, two or more VL CDRs listed in Table 1. In particular, the invention encompasses the use of an antibody that immunospecifically binds to integrin αᵥβ₃, said antibody comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3, or any combination thereof of the VH CDRs and VL CDRs listed in Table 1 supra.

In one embodiment, an antibody that immunospecifically binds to integrin αᵥβ₃ comprises a VH CDR1 having the amino acid sequence of SEQ ID NO:1 and a VL CDR1 having the amino acid sequence of SEQ ID NO:4. In another embodiment, an antibody that immunospecifically binds to integrin αᵥβ₃ comprises a VH CDR1 having the amino acid sequence of SEQ ID NO:1 and a VL CDR2 having the amino acid sequence of SEQ ID NO:5. In another embodiment, an antibody that immunospecifically binds to integrin αᵥβ₃ comprises a VH CDR1 having the amino acid sequence of SEQ ID NO:1 and a VL CDR3 having the amino acid sequence of SEQ ID NO:6.

In another embodiment, an antibody that immunospecifically binds to integrin αᵥβ₃ comprises a VH CDR2 having the amino acid sequence of SEQ ID NO:2 and a VL CDR1 having the amino acid sequence of SEQ ID NO:4. In another embodiment, an antibody that immunospecifically binds to integrin αᵥβ₃ comprises a VH CDR2 having the amino acid sequence of SEQ ID NO:2 and a VL CDR2 having the amino acid sequence of SEQ ID NO:5. In another embodiment, an antibody that immunospecifically binds to integrin αᵥβ₃ comprises a VH CDR2 having the amino acid sequence of SEQ ID NO:2 and a VL CDR3 having the amino acid sequence of SEQ ID NO:6.

In another embodiment, an antibody that immunospecifically binds to integrin αᵥβ₃ comprises a VH CDR3 having the amino acid sequence of SEQ ID NO:3 and a VL CDR1 having the amino acid sequence of SEQ ID NO:4. In another embodiment, an antibody that immunospecifically binds to integrin αᵥβ₃ comprises a VH CDR3 having the amino acid sequence of SEQ ID NO:3 and a VL CDR2 having the amino acid sequence of SEQ ID NO:5. In a preferred embodiment, an antibody that immunospecifically binds to integrin αᵥβ₃ comprises a VH CDR3 having the amino acid sequence of SEQ ID NO:3 and a VL CDR3 having the amino acid sequence of SEQ ID NO:6.

The present invention encompasses the use of a nucleic acid molecule(s), generally isolated, encoding an antibody that immunospecifically binds to integrin αᵥβ₃. In a specific embodiment, an isolated nucleic acid molecule(s) encodes an antibody that immunospecifically binds to integrin αᵥβ₃, said antibody having the amino acid sequence of LM609 or VITAXIN®.

In one embodiment, an isolated nucleic acid molecule encodes an antibody that immunospecifically binds to integrin αᵥβ₃, said antibody comprising a VH domain having the amino acid sequence of the VH domain of LM609 or VITAXIN®. In another embodiment, an isolated nucleic acid molecule encodes an antibody that immunospecifically binds to integrin αᵥβ₃, said antibody comprising a VH domain having the amino acid sequence of the VH domain of the monoclonal antibody produced by the cell line deposited with the ATCC™ as Accession Number HB 9537. In another embodiment, an isolated nucleic acid molecule encodes an antibody that immunospecifically binds to integrin αᵥβ₃, said antibody comprising a VH CDR1 having the amino acid sequence of the VH CDR1 listed in Table 1. In another embodiment, an isolated nucleic acid molecule encodes an antibody that immunospecifically binds to integrin αᵥβ₃, said antibody comprising a VH CDR2 having the amino acid sequence of the VH CDR2 listed in Table 1. In yet another embodiment, an isolated nucleic acid molecule encodes an antibody that immunospecifically binds to integrin αᵥβ₃, said antibody comprising a VH CDR3 having the amino acid sequence of the VH CDR3 listed in Table 1.

In one embodiment, an isolated nucleic acid molecule encodes an antibody that immunospecifically binds to integrin αᵥβ₃, said antibody comprising a VL domain having the amino acid sequence of the VL domain of LM609 or VITAXIN®. In another embodiment, an isolated nucleic acid molecule encodes an antibody that immunospecifically binds to integrin αᵥβ₃, said antibody comprising a VL domain having the amino acid sequence of the VL domain of the monoclonal antibody produced by the cell line deposited with the ATICC™ as Accession Number HB 9537. In another embodiment, an isolated nucleic acid molecule encodes an antibody that immunospecifically binds to integrin αᵥβ₃, said antibody comprising a VL CDR1 having the amino acid sequence of the VL CDR1 listed in Table 1. In another embodiment, an isolated nucleic acid molecule encodes an antibody that immunospecifically bind to integrin αᵥβ₃, said antibody comprising a VL CDR2 having the amino acid sequence of the VL CDR2 listed in Table 1. In yet another embodiment, an isolated nucleic acid molecule encodes an antibody that immunospecifically binds to integrin αᵥβ₃, said antibody comprising a VL CDR3 having the amino acid sequence of the VL CDR3 listed in Table 1.

In another embodiment, an isolated nucleic acid molecule encodes an antibody that immunospecifically binds to integrin αᵥβ₃, said antibody comprising a VH domain having the amino acid sequence of the VH domain of LM609 or VITAXIN® and a VL domain having the amino acid sequence of the VL domain of LM609 or VITAXIN®. In another embodiment, an isolated nucleic acid molecule encodes an antibody that immunospecifically binds to integrin αᵥβ₃, said antibody comprising a VH CDR1, a VL CDR1, a VH CDR2, a VL CDR2, a VH CDR3, a VL CDR3, or any combination thereof having an amino acid sequence listed in Table 1.

The present invention encompasses the use of antibodies that immunospecifically bind to integrin αᵥβ₃, said antibodies comprising derivatives of the VH domains, VH CDRs, VL domains, or VL CDRs described herein that immunospecifically bind to integrin αᵥβ₃. Standard techniques known to those of skill in the art can be used to introduce mutations (e.g., substitutions, deletions and/or additions) in the nucleotide sequence encoding an antibody of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues (e.g., amino acid residues which are not critical for the antibody to immunospecifically bind to integrin αᵥβ₃). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains ( e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

The present invention encompasses the use of antibodies that immunospecifically bind to integrin αᵥβ₃, said antibodies comprising the amino acid sequence of LM609 or VITAXIN® with one or more amino acid residue substitutions in the variable light (VL) domain and/or variable heavy (VH) domain. The present invention encompasses the use of antibodies that immunospecifically bind to integrin αᵥβ₃, said antibodies comprising the amino acid sequence of LM609 or VITAXIN® with one or more amino acid residue substitutions in one or more VL CDRs and/or one or more VH CDRs. The antibody generated by introducing substitutions in the VH domain, VH CDRs, VL domain and/or VL CDRs of LM609 or VITAXIN® can be tested in vitro and in vivo, for example, for its ability to bind to integrin αᵥβ₃ (by, e.g., immunoassays including, but not limited to ELISAs and BIAcore), or for its ability to prevent, manage, treat or ameliorate one or more symptoms associated with an inflammatory disease, an autoimmune disorder, a disorder associated with aberrant expression and/or activity of integrin αᵥβ₃, a disorder associated with abnormal bone metabolism, a disorder associated with aberrant angiogenesis or cancer.

In a specific embodiment, an antibody that immunospecifically binds to integrin αᵥβ₃ comprises a nucleotide sequence that hybridizes to the nucleotide sequence encoding the monoclonal antibody produced by the cell line deposited with the ATCC™ as Accession Number HB 9537 under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g. hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

In another embodiment, an antibody that immunospecifically binds to integrin αᵥβ₃ comprises a nucleotide sequence that hybridizes to the nucleotide sequence encoding the LM609 or VITAXIN® under stringent conditions. In another embodiment, an antibody that immunospecifically binds to integrin αᵥβ₃ comprises an amino acid sequence of a VH domain or an amino acid sequence a VL domain encoded by a nucleotide sequence that hybridizes to the nucleotide sequence encoding the VH or VL domains of LM609 or VITAXIN® under stringent conditions. In another embodiment, an antibody that immunospecifically binds to integrin αᵥβ₃ comprises an amino acid sequence of a VH CDR or an amino acid sequence of a VL CDR encoded by a nucleotide sequence that hybridizes to the nucleotide sequence encoding any one of the VH CDRs or VL CDRs listed in Table 1 under stringent conditions.

In another embodiment, an antibody that immunospecifically binds to integrin αᵥβ₃ comprises an amino acid sequence of a VH CDR or an amino acid sequence of a VL CDR encoded by a nucleotide sequence that hybridizes to the nucleotide sequence encoding any one of VH CDRs or VL CDRs of the monoclonal antibody produced by the cell line deposited with the ATCC™ as Accession Number HB 9537 under stringent conditions. In another embodiment, an antibody that immunospecifically binds to integrin αᵥβ₃ comprises an amino acid sequence of a VH CDR and an amino acid sequence of a VL CDR encoded by nucleotide sequences that hybridizes to the nucleotide sequences encoding any one of the VH CDRs and VL CDRs listed in Table 1 under stringent conditions. In another embodiment, an antibody that immunospecifically binds to integrin αᵥβ₃ comprises an amino acid sequence of a VH CDR and an amino acid sequence of a VL CDR encoded by nucleotide sequences that hybridizes to the nucleotide sequences encoding the monoclonal antibody produced by the cell line deposited with the ATCC™ as Accession Number HB 9537 under stringent conditions.

In a specific embodiment, an antibody that immunospecifically binds to integrin αᵥβ₃ comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of the monoclonal antibody produced by the cell line deposited with the ATCC™ as Accession Number HB 9537. In another embodiment, an antibody that immunospecifically binds to integrin αᵥβ₃ comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of VITAXIN®. The determination of percent identity of two amino acid sequences can be determined by any method known to one skilled in the art, including BLAST protein searches. See Section 3.1 supra for a brief description regarding methods for determining percent identity of two amino acid sequences.

In another embodiment, an antibody that immunospecifically binds to integrin αᵥβ₃ comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of VITAXIN®. In another embodiment, an antibody that immunospecifically binds to integrin αᵥβ₃ comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of the monoclonal antibody produced by the cell line deposited with the ATCC™ as Accession Number HB 9537.

In another embodiment, an antibody that immunospecifically binds to integrin αᵥβ₃ comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VH CDRs listed in Table 1. In another embodiment, an antibody that immunospecifically binds to integrin αᵥβ₃ comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of one of the VH CDRs of the monoclonal antibody produced by the cell line deposited with the ATCC™ as Accession Number HB 9537.

In another embodiment, an antibody that immunospecifically binds to integrin αᵥβ₃ comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of VITAXIN®. In another embodiment, an antibody that immunospecifically binds to integrin αᵥβ₃ comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of the monoclonal antibody produced by the cell line deposited with the ATCC™ as Accession Number HB 9537.

In another embodiment, an antibody that immunospecifically binds to integrin αᵥβ₃ comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 1. In another embodiment, an antibody that immunospecifically binds to integrin αᵥβ₃ comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs of the monoclonal antibody produced by the cell line deposited with the ATCC™ as Accession Number HB 9537.

The present invention encompasses antibodies that compete with an antibody described herein for binding to integrin αᵥβ₃. In a specific embodiment, the present invention encompasses antibodies that compete with LM609 or an antigen-binding fragment thereof for binding to integrin αᵥβ₃. In a preferred embodiment, the present invention encompasses antibodies that compete with VITAXIN® or an antigen-binding fragment thereof for binding to integrin αᵥβ₃. In an alternate embodiment, the invention does not emcompass an antibody or fragment thereof that competes with VITAXIN® or a fragment thereof for binding to integrin αᵥβ₃. In yet another embodiment, the invention does not encompass VITAXIN® or an antigen-binding fragment thereof and/or an antibody or fragment that competes with VITAXIN® or a fragment thereof for binding to integrin αᵥβ₃. Antibodies or fragments thereof that compete with VITAXIN® or an antigen-binding fragment thereof can be identified using techniques well-known to one of skill in the art, including but not limited to competitive immunoassays.

The present invention also encompasses VH domains that compete with the VH domain of LM609 or VITAXIN® for binding to integrin αᵥβ₃. The present invention also encompasses VL domains that compete with a VL domain of LM609 or VITAXIN® for binding to integrin αᵥβ₃. The present invention also encompasses VH CDRs that compete with a VH CDR listed in Table 1 for binding to integrin αᵥβ₃, or a VH CDR of the monoclonal antibody produced by the cell line deposited with the ATCC™ as Accession Number HB 9537 for binding to integrin αᵥβ₃. The present invention also encompasses VL CDRs that compete with a VL CDR listed in Table 1 for binding to integrin αᵥβ₃, or a VL CDR of the monoclonal antibody produced by the cell line deposited with the ATCC™ as Accession Number HB 9537 for binding to integrin αᵥβ₃

Antibodies that immunospecifically bind to integrin αᵥβ₃ include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides antibodies that immunospecifically bind to integrin αᵥβ₃, said antibodies comprising a framework region known to those of skill in the art and preferably one or more of the VH CDRs and/or VL CDRs described herein. Preferably, the fragment region of an antibody of the invention is human. In a specific embodiment, an antibody that immunospecifically binds to integrin αᵥβ₃ comprises the framework region of VITAXIN®.

The present invention also encompasses antibodies which immunospecifically bind to integrin αᵥβ₃, said antibodies comprising the amino acid sequence of VITAXIN® with one or more mutations (e.g., one or more amino acid substitutions) in the framework regions. In certain embodiments, antibodies which immunospecifically bind to integrin αᵥβ₃ comprise the amino acid sequence of VITAXIN® with one or more amino acid residue substitutions in the framework regions of the VH and/or VL domains.

The present invention also encompasses antibodies which immunospecifically bind to integrin αᵥβ₃, said antibodies comprising the amino acid sequence of VITAXIN® with one or more mutations (e.g., one or more amino acid residue substitutions) in the variable and framework regions.

The present invention encompasses the use of fusion proteins comprising an antibody that immunospecifically binds to integrin αᵥβ₃ and a heterologous polypeptide. Preferably, the heterologous polypeptide that the antibody is fused to is useful for targeting the antibody to platelets, monocytes, macrophages, endothelial cells, osteoclasts, activated T cells, and/or B cells.

4.1.1.1 Antibodies Having Increased Half-Lives that Immunospecifically Bind to Integrin to αᵥβ₃

The present invention provides for antibodies that immunospecifically bind to integrin αᵥβ₃ which have a extended half-life in vivo. In particular, the present invention provides antibodies that immunospecifically bind to integrin αᵥβ₃ which have a half-life in an animal, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies, single chain antibodies and Fab fragments) in vivo, for example, inert polymer molecules such as high molecular weight polyethyleneglycol (PEG) can be attached to the antibodies with or without a multifunctional linker either through site-specific conjugation of the PEG to the N— or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication Nos. WO 98/23289; and WO 97/34631; and U.S. Pat. No. 6,277,375, each of which is incorporated herein by reference in its entirety.

Further, antibodies can be conjugated to albumin in order to make the antibody or antibody fragment more stable in vivo or have a longer half-life in vivo. The techniques well-known in the art, see, e.g., International Publication Nos. WO 93/15199, WO 93/15200, and WO 01/77137, and European Patent No. EP 413,622, all of which are incorporated herein by reference in their entirety.

4.1.1.2 Antibody Conjugates

The present invention encompasses antibodies or fragments thereof that immunospecifically binds to integrin αᵥβ₃ recombinantly fused or chemically conjugated (including both covalent and non-covalent conjugations) to a heterologous moiety (e.g., a heterologous protein, polypeptide, peptide, antibody or antibody fragment, a marker sequence, a diagnostic agent, a therapeutic moiety, a radioactive metal ion, a polymer, albumin, or a solid support). In particular, the present invention encompasses antibodies or fragments thereof that immunospecifically binds to integrin αᵥβ₃ recombinantly fused or chemically conjugated (including both covalent and non-covalent conjugations) to a heterologous polypeptide or protein or fragment thereof (preferably to a polypepetide of at least 10, at least 20, at least 30, at least 40, at least 50, at least 60, at least 70, at least 80, at least 90 or at least 100 amino acids), to generate fusion proteins. The invention encompasses fusion proteins comprising an antigen-binding fragment of an antibody described herein (e.g., a Fab fragment, Fd fragment, Fv fragment, F(ab)2 fragment, a VH domain, a VH CDR, a VL domain or a VL CDR) and a heterologous polypeptide, protein, or peptide. Preferably, the heterologous polypeptide that the antibody or antibody fragment is fused to is useful for targeting the antibody to endothelial cells, B cells, osteoclasts or activated T cells. For example, an antibody that immunospecifically binds to a cell surface receptor expressed by a particular cell type (e.g., an endothelial cell, a B cell, an osteoclast, or an activated T cell) may be fused or conjugated to an antibody or fragment of the invention. Methods for fusing or conjugating polypeptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references incorporated by reference in their entireties).

Additional fusion proteins of anti-integrin αᵥβ₃ antibodies, may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33 ; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that immunospecifically binds to integrin αᵥβ₃ may be recombined with one or more components, motifs, sections, parts, domains, fragments, etc. of one or more heterologous molecules.

Antibodies that immunospecifically bind to integrin αvβ3 or fragments thereof can be fused to marker sequences, such as a peptide to facilitate purification. In specific embodiments, the marker amino acid sequence is a hexa-histidine peptide, such as the tag provided in a pQE vector (QIAGEN™, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies of the present invention or fragments or variants thereof conjugated to a diagnostic or detectable agent. Such antibodies can be useful for monitoring or prognosing the development or progression of a disease or disorder as part of a clinical testing procedure, such as determining the efficacy of a particular therapy. Such diagnosis and detection can be accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I,), carbon (¹⁴C), sulfur (³⁵S), tritium (³H), indium (¹¹⁵In, ¹¹³In, ¹¹²In, ¹¹¹In,), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹yb, ⁵¹Cr, ⁵⁴Mn, ⁷⁵Se, ¹¹³Sn, and ¹¹⁷Tin; positron emitting metals using various positron emission tomographies, noradioactive paramagnetic metal ions, and molecules that are radiolabelled or conjugated to specific radioisotopes.

The present invention further encompasses uses of antibodies or fragments thereof conjugated to a therapeutic moiety that has a prophylactic or therapeutic effect(s) when targeted to a particular site by way of a targeting agent such as an antibody. An antibody or fragment thereof may be conjugated to a therapeutic moiety such as a cytotoxin, e.g., a cytostatic or cytocidal agent, or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference), hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)), cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459), farnesyl transferase inhibitors (e.g., R115777, BMS-214662, and those disclosed by, for example, U.S. Pat. Nos 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305), topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin; bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate); HMG-CoA reductase inhibitors, (e.g., lovastatin, simvastatin, atorvastatin, pravastatin, fluvastatin, statin, cerivastatin, lescol, lupitor, rosuvastatin and atorvastatin) and pharmaceutically acceptable salts, solvates, clathrates, and prodrugs thereof. See, e.g., Rothenberg, M. L., Annals of Oncology 8:837-855(1997); and Moreau, P., et al., J. Med. Chem. 41:1631-1640(1998)), antisense oligonucleotides (e.g., those disclosed in the U.S. Pat. Nos. 6,277,832, 5,998,596, 5,885,834, 5,734,033, and 5,618,709), immunomodulators (e.g., antibodies and cytokines), antibodies, and adenosine deaminase inhibitors (e.g., Fludarabine phosphate and 2-Chlorodeoxyadenosine).

An antibody that immunnospecifically binds to integrin αvβ3 or a fragment thereof may be conjugated to a therapeutic moiety or drug moiety that modifies a given biological response. Therapeutic or drug moieties are not to be construed as limited to classical chemical therapeutic agents. For example, the drug moiety may be a protein, polypeptide or peptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, γ-interferon, nerve growth factor, platelet derived growth factor, tissue plasminogen activator, an apoptotic agent, e.g., TNF-α, TNF-β, AIM I (see, International publication No. WO 97/33899), AIM II (see, International Publication No. WO 97/34911), Fas Ligand (Takahashi et al., 1994, J. Immunol., 6:1567-1574), and VEGF (see, International publication No. WO 99/23105); or, a biological response modifier such as, for example, a lymphokine (e.g., interleukin-1 (“IL-1”), interleukin-2 (“IL-2”), interleukin-4 (“IL-4”), interleukin-6 (“IL-6), interleukin-9 (“IL-9”), interleukin-10 (“IL-10”), interleukin-12 (“IL-12”), interleukin-23 (“IL-23”), granulocyte macrophage colony stimulating factor (“GM-CSF”), and granulocyte colony stimulating factor (“G-CSF”)), and a growth factor (e.g., growth hormone (“GH”)).

Alternatively, an antibody that immunospecifically binds to integrin αvβ3 or a fragment thereof can also be conjugated to a second antibody to form an antibody heteroconjugate as described by Segal in U.S. Pat. No. 4,676,980, which is incorporated herein by reference in its entirety.

Moreover, an antibody that immunospecifically binds to integrin αvβ3 or a fragment thereof can be conjugated to a therapeutic moiety such as a radioactive metal ion (including but not limited to, ²¹³Bi, ¹³¹In, ¹³¹LU, ¹³¹Y, ¹³¹Ho, and ¹³¹Sm, to polypeptides utilizing macrocyclic chelators. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Arnon et al., “Monoclonal Antibodies for Immunotargeting of Drugs In Cancer Therapy”, in Monoclonal Antibodies and Cancer Therapy, Reisfeld et al. (eds.), pp. 243-56 (Alan R. Liss, Inc. 1985); Hellstrom et al., “Antibodies for Drug Delivery”, in Controlled Drug Delivery (2nd Ed.), Robinson et al. (eds.), pp. 623-53 (Marcel Dekker, Inc. 1987); Thorpe, “Antibody Carriers of Cytotoxic Agents In Cancer Therapy: A Review”, in Monoclonal Antibodies 84: Biological and Clinical Applications, Pinchera et al. (eds.), pp. 475-506 (1985); “Analysis, Results, and Future Prospective of The Therapeutic Use of Radiolabeled Antibody In Cancer Therapy”, in Monoclonal Antibodies For Cancer Detection And Therapy, Baldwin et aL (eds.), pp. 303-16 (Academic Press 1985), and Thorpe et al., 1982, Immunol. Rev. 62:119-58.

The therapeutic moiety or drug conjugated to an antibody or fragment thereof that immunospecifically binds to integrin αᵥβ₃ should chosen to achieve the desired prophylactic or therapeutic effect(s) for a particular disorder in a subject. A clinician or other medical personnel should consider the following when deciding on which therapeutic moiety or drug to conjugate to an antibody or fragment thereof that immunospecifically binds to integrin αᵥβ₃: the nature of the disease, the severity of the disease, and the condition of the subject.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Therapeutic moieties or drugs such as those described herein can also be conjugated to integrin αᵥβ₃ antagonists other than antibodies using techniques well-known in the art.

4.2 Methods of Identifying Antibodies that Immunospecifically Bind to Integrin αᵥβ₃.

The invention encompasses methods for identifying antagonists that are immunospecific for integrin α_{V}β₃, particularly for antibodies that specifically bind to the same epitope as VITAXIN® and/or LM609. This aspect of the invention is based, in part, on the discovery that mutation of residues 171, 173 and/or 174 of the human β₃ chain disrupt binding of VITAXIN® and/or LM609 antibodies to the integrin α_{V}β₃ heterodimer. This aspect of the invention is also based, in part, on the discovery that although VITAXIN® and LM609 do not bind to mouse integrin α_{V}β₃, VITAXIN® and LM609 do bind to a modified mouse integrin α_{V}β₃ in which the region of the mouse β chain that corresponds to amino acids 164-202 of the human β chain are replaced with amino acids 164-202 of the human β chain. In certain embodiments, amino acid substitutions are made in the subunits of integrin α_{V}β₃, for example to change the ligand specificity of the integrin α_{V}β₃ and/or disrupt the heterodimerization of the subunit chains. Preferably the integrin α_{V}β₃ is human. In specific embodiments, such amino acid substitutions disrupt the specific interaction of certain antagonists of integrin α_{V}β₃ with a particular integrin α_{V}β₃ epitope. In a preferred embodiment, the amino acid substitutions are made within regions of an integrin subunit that confers ligand binding specificity, preferably ligand binding specificity of LM609 and/or VITAXIN®, particularly residues 164-202 of human β₃. Alternatively, mouse β chain residues corresponding to residues 164-202 of the human β₃ chain are replaced with the residues 164-202 of the human β₃ chain. Such mouse-human chimeras can be used to screen for antagonists that bind to the region 164-202 of human β₃ but not to mouse integrin α_{V}β₃.

In preferred embodiments, the amino acid substitutions are made in the β₃ subunit. In certain embodiments, human β₃ residues are substituted with rat residues as described in Table 2. In one embodiment, the substitution of human residue Glu to rat residue Gln at position 171 (“Mutation A”) disrupts integrin α_{V}β₃ binding to LM609. This same change disrupts binding to VITAXIN®. In another embodiment, the substitution of human residue Leu and Glu to rat residues Ilc, and Lys at positions 173 and 174, respectively (“Mutation B”) both disrupt binding to VITAXIN® and increase binding to an anti rat β₃ antibody. In yet another embodiment, the substitution of human residues Asp and Thr to rat residues Thr and Ser at positions 179 and 182 respectively (“Mutation C”) confer binding specificity to an anti-rat β₃ antibody. Mutations A and C combined (three substituted residues) confer binding specificity for the mouse-anti-rat β₃ antibody and disrupts binding to VITAXIN®. In a specific preferred embodiment, amino acids 171, 173 and 174 can be substituted to disrupt binding to VITAXIN®. In an alternate preferred embodiment, amino acids 171, 173, 174, 179 and 182 can be substituted to disrupt binding of integrin α_{V}β₃ to LM609 and humanized anti-integrin α_{V}β₃ antibodies such as VITAXIN®. Such substitutions preferred examples but not limiting. Such substituted subunits are merely exemplary and not limiting. Any integrin α_{V}β₃ regions identified to be responsible for antibody binding can be altered with substituted, deleted or inserted residues to characterize binding specificity of various antibodies and to screen for antibodies with the same a similar binding specificity.

Amino acid substituted subunits of integrin α_{V}β₃ can be used for screening antibodies with specific affinity for particular epitopes by identifying monoclonal antibodies that bind to wild type integrin α_{V}β₃ but not the altered form, or that bind mouse α_{V}β₃ integrins with a region substituted with the corresponding region from the human α_{V}β₃ but do not bind to wild type mouse integrin α_{V}β₃. In addition, the invention provides methods for identifying monoclonal antibodies that bind to the heterodimerized α_{V}β₃ but not the α_{V} or the β₃ chains when not included in a heterodimer. Such screening can be accomplished by any routine method for assaying antibody specificity known in the art, for example, using cell lines that do not express wild type integrin α_{V}β₃ to recombinantly express the mutant α_{V}β₃ or individual α_{V} or β₃ chains. The antibodies identified from such screening methods can be useful for the prevention, management and treatment of integrin α_{V}β₃-mediated diseases and disorders, including but not limited to inflammatory diseases, autoimmune disorders, disorders associated with aberrant expression and/or activity of integrin αᵥβ₃, disorders associated with abnormal bone metabolism, disorders associated with aberrant angiogenesis and cancers, or a condition or symptom associated therewith. It is also contemplated that such antibodies can be used in the methods and compositions contemplated by the present invention. Preferably, these antibodies are not LM609, VITAXIN® or an antibody or antibody binding fragment thereof having the CDRs (or one, two, three, four or five of the CDRs or CDR3 of the heavy chain) of LM609 or VITAXIN® with no more than one, no more than two, no more than five, no more than eight, or no more than ten amino acid substitutions, deletions or insertions.

TABLE 2

| Human Beta3 | Mutation A | Mutation B | | Mutation C | |
|---|---|---|---|---|---|
| mutants | (Glu-Gln) | (Leu-Ile), (Glu-Lys) | | (Asp-Thr), (Thr-Ser) | |
| A1(A, C) | E171Q | | | D179T | T182S |
| A6 | E171Q | | | | |
| B1 | | L173 I | E174K | | |
| C14 | | | | D179T | T182S |
| C16 | | | | D179T | T182S |
| ABC17 | E171Q | L173 I | E174K | D179T | T182S |

4.3 Bisphosphonates

Bisphosphonates are potent antiresorptive drugs that directly inhibit osteoclast activity, thus reducing bone resorption. These drugs increase bone mass and decrease the risk of fractures in patients with bone metabolism disorders. In addition, at high doses, bisphosphonates have been shown to have antiproliferative and proapoptotic effects. See e.g., Green, J., 2003, Cancer 97 (3 Suppl):840-7. Any bisphosphonate well-known to one of skill in the art can be used in the compositions and the methods of the invention. Non-limiting examples of bisphosphonates which can be used in accordance with the invention include alendronate, cimadronte, clodronate, tiludronate, etidronate, ibandronate, neridronate, olpandronate, risedronate, piridronate, pamidronate, zolendronate, pharmaceutically acceptable salts thereof, and mixtures thereof. In a preferred embodiment, a method of treating a disorder associated with or characterized by abnormal bone resorption comprises administering to a subject in need of treatment, a glucocorticoid in combination with an integrin αᵥβ₃ antagonist and a bisphosphonate.

Bisphosphonates and their dosages, routes of administration and recommended usage are known in the art and have been described in such literature as the Physician's Desk Reference (57^{th} ed., 2003). Non-limiting examples of bisphosphonates and their doses and methods of administration are summarized below in Table 2.

TABLE 2

| Therapeutic Agent | Dose/Administration/Formulation | | |
|---|---|---|---|
| alendronate sodium | Oral (tablets) | Tablets contain 6.53, | Treatment of |
| Fosomax ® (Merck) | | 13.05, 45.68, 52.21, or | osteoporosis (one 70 mg |
| | | 91.37 mg of | tablet weekly or one |
| | | alendronate | 10 mg tablet once |
| | | monosodium salt | daily). |
| | | trihydrate (molar | Prevention of |
| | | equivalent of 5.0, 10.0, | osteoporosis (one 35 mg |
| | | 35, 40, and 70 mg | tablet once weekly |
| | | respectively, of free | or one 5 mg tablet once |
| | | acid) | daily) |
| | | | Treatment of |
| | | | glucocorticoid-induced |
| | | | osteoporosis (one 5 mg |
| | | | tablet daily or one 10 mg |
| | | | tablet daily for |
| | | | postmenopausal women |
| | | | not receiving estrogen |
| | | | therapy) |
| | | | Paget's disease (40 mg |
| | | | once daily for six |
| | | | months) |
| disodium etidronate | Oral (tablet) taken daily | 200 mg or 400 mg | Heterotropic |
| e.g., Didronel ™ | | tablets | ossification (aka |
| (Procter and Gamble) | | | myositus ossificans, |
| | | | ectopic calcification, |
| | | | peroarticular |
| | | | ossification, |
| | | | paraosteoarthropathy) |
| | | | Total hip replacment |
| | | | patients (20 mg/kg/day |
| | | | for 1 month before and |
| | | | 3 months after surgery) |
| | | | Spinal cord injured |
| | | | patients (20 mg/kg/day |
| | | | for 2 weeks folowed by |
| | | | 10 mg/kg/day for 10 |
| | | | weeks) |
| | | | Paget's disease (5 to 10 |
| | | | mg/kg/day, not to |
| | | | exceed 6 months, or 11 |
| | | | to 20 mg/kg/day not to |
| | | | exceed 3 months) |
| | | | Osteoporosis (90 day |
| | | | cycles of 400 mg/day |
| | | | for 14 days then 1.25 g/day |
| | | | calcium carbonate |
| | | | (e.g., Cacit ®) for 76 |
| | | | days). |
| tiludronic acid | Oral (tablet) | Tablets contain 240 mg | Paget's disease (400 mg |
| (tiludronate disodium) | | tiludronate disodium | once daily for 3 |
| e.g., Skelid ® (Sanofi- | | (molar equivalent of | months, repeated if |
| Synthelabo) | | 200 mg tiludonic acid) | necessary after 6 |
| | | | months) |
| sodium clodronate | Oral (tablet) or | 400 or 800 mg tablets | Treatment of osteolytic |
| e.g., Bonefos ™ | intravenous | or | lesions, hypercalcemia |
| (Boehringer Ingelheim) | (concentrate) | 60 mg/mL concentrate | and bone pain |
| | | for dilution and use as | associated with breast |
| | | infusion | cancer or multiple |
| | | | myeloma (one 1.6 g/day |
| | | | oral dose or 3.2 g/day |
| | | | divided into two oral |
| | | | doses) |
| | | | Treatment of |
| | | | hypercalcaemia of |
| | | | malignancy (slow |
| | | | intravenous infusion |
| | | | 300 mg daily for |
| | | | maximum 7-10 days, or |
| | | | by single-dose infusion |
| | | | of 1.5 g) |
| clodronate disodium | Oral (tablet) | | I.V.: Single Infusion: |
| Ostac ® | | | Recommended Dosage: |
| (Roche) | | | The contents of five 10 mL |
| | | | ampuls is |
| | | | administered by slow |
| | | | i.v. infusion over a |
| | | | period of not less than 4 |
| | | | hours. |
| | | | Administration: Five 10 mL |
| | | | ampuls is diluted |
| | | | with 500 mL of 0.9% |
| | | | w/v sodium chloride |
| | | | injections, USP or 5% |
| | | | w/v dextrose, USP and |
| | | | administered by slow |
| | | | i.v. infusion over a |
| | | | period of not less than 4 |
| | | | hours. |
| | | | Multiple Infusions: |
| | | | Recommended Dosage: |
| | | | The contents of 1 ampul |
| | | | administered once daily |
| | | | over 2 to 6 hours. |
| | | | Administration: 1 |
| | | | ampul is diluted with |
| | | | 500 mL of 0.9% w/v |
| | | | sodium chloride |
| | | | injection, USP or 5% |
| | | | w/v dextrose, USP and |
| | | | administered by slow |
| | | | i.v. infusion over 2 to 6 |
| | | | hours |
| sodium clodronate | Oral (tablet) | 400 or 520 mg tablets | Treatment of osteolytic |
| Loron ® (Roche) | | (N.B. due to greater | lesions, hypercalcemia |
| Loron 520 ® (Roche) | | bioavailability of 520 mg | and bone pain |
| | | Loron 520 ® | associated with breast |
| | | formulation, tablet is | cancer or multiple |
| | | equivalent to two 400 mg | myeloma (one 1.6 g/day |
| | | Loron ® tablets) | oral dose or 3.2 g/day |
| | | | divided into two oral |
| | | | doses) |
| disodium pamidronate | Intravenous | 30 or 90 mg vials | 30, 60 or 90 mg |
| Aredia ™ (Novartis) | | (containing 30 mg and | administered over 4 |
| | | 90 mg of sterile | hours, or 90 mg |
| | | lyophilized | administered over 24 |
| | | pamidronate disodium | hours to treat bone |
| | | respectively) | metastases of breast |
| | | | cancer and osteolytic |
| | | | lesions of multiple |
| | | | myeloma, |
| | | | hypercalcemia of |
| | | | malignancy, Paget's |
| | | | disease |
| ibandronate | Oral (tablet) | Under development by | Treatment of |
| (under development by | | Roche and | osteoporosis (2.5 mg |
| Roche and | | GlaxoSmithKline | oral ibandronate daily |
| GlaxoSmithKline) | | | or 20 mg oral |
| | | | ibandronate taken every |
| | | | other day for 24 days |
| | | | followed by a drug-free |
| | | | interval of up to nine to |
| | | | ten weeks, in |
| | | | combination with daily |
| | | | oral calcium (500 mg) |
| | | | and vitamin D |
| | | | (400IU)). |
| zoledronic acid | Intravenous (powder for | 4 mg vial | Treatment of |
| (Zometa ™) | re-constitution) | | hypercalcaemia of |
| | | | malignancy (4 mg as |
| | | | single dose by |
| | | | intravenous infusion) |
| risedronate sodium | Oral (tablet) | Each tablet contains the | Treatment of |
| Actonel ® | | equivalent of 5 or 30 mg | osteoporosis (5 mg |
| | | of anhydrous | once daily, optionally in |
| | | risedronate sodium | combination with 0.625 mg |
| | | | estrogen daily) |
| | | | Prevention of |
| | | | glucocorticoid-induced |
| | | | osteoporosis (5 mg |
| | | | once daily) |
| | | | Treatment of Paget's |
| | | | disease (30 mg daily for |
| | | | 2 months) |

4.4 HMG-CoA Reductase Inhibitors

HMG-CoA reductase inhibitors are generally used to decrease the risk of bone fractures, treat elevated cholesterol levels and disorders associated therewith. Any HMG-CoA reductase inhibitor well-known to one of skill in the art can be used in the compositions and the methods of the invention. Non-limiting examples of HMG-CoA reductase inhibitors which can be used include lovastatin, simvastatin, atorvastatin, pravastatin, fluvastatin, statin, cerivastatin, lescol, lupitor, rosuvastatin and atorvastatin.

HMG-CoA reductase inhibitors and their dosages, routes of administration and recommended usage are known in the art and have been described in such literature as the Physician's Desk Reference (57^{th} ed., 2003). Non-limiting examples of HMG-CoA reductase inhibitors and their doses and methods of administration are summarized below in Table 3.

TABLE 3

| Therapeutic Agent | Dose/Administration/Formulation | | |
|---|---|---|---|
| atorvastatin calcium | Oral (tablet) | 10, 20, 40 or 80 mg | Treatment of |
| Lipitor ™ (Pfizer) | | tablets | hypercholesterolemia or |
| | | | homozygous familial |
| | | | hypercholesterolemia |
| | | | (range 10-80 mg daily, |
| | | | recommended starting |
| | | | dosage 10 or 20 mg |
| | | | once daily, if needed |
| | | | raise to 40 mg daily) |
| pravastatin sodium | Oral (tablet) | 10, 20 or 40 mg tablets | Treatment of |
| Pravachol ™ (Bristol- | | | hypercholesterolemia |
| Myers Squibb) | | | (recommended starting |
| | | | range 10-40 mg daily) |
| lovastatin | Oral (tablet) | 10, 20, or 40 mg tablets | Treat elevated |
| Mevacor ® (Merck) | | | cholesterol levels |
| | | | (dosage range 10-80 mg/day, |
| | | | recommended |
| | | | starting dose 20 mg |
| | | | once daily) |
| simvastatin | Oral (tablet) | 5, 10, 20, 40 or 80 mg | Dosage range of 5-80 mg/day. |
| Zocor ® | | tablets | Treatment of patients |
| (Merck) | | | with homozygous |
| | | | familial |
| | | | hypercholesterolemia |
| | | | (40 mg in evening or 80 mg/day |
| | | | in 3 divided |
| | | | doses of 20 mg, 20 mg |
| | | | and an evening dose of |
| | | | 40 mg) |
| fluvastatin | Oral (capsules or | 20 or 40 mg capsules or | Recommended staring |
| (fluvastatin sodium) | extended release | 80 mg tablets extended | dosage is 40 mg as one |
| Lescol ® | tablets) | release formulation | capsule once a day, one |
| (Novartis) | | | 80 mg extended release |
| | | | tablet in the evening |
| | | | once a day, or 80 mg in |
| | | | divided doses of the 40 mg |
| | | | capsule given twice |
| | | | a day |

Other Agents Used in Combination With Integrin αᵥβ₃ Antagonist-Bisphosphonate and Integrin αᵥβ₃ Antagonist-HMG-CoA Reductase Combinations

The present invention provides methods for preventing, treating, managing, or ameliorating inflammatory diseases, autoimmune disorders, disorders associated with aberrant expression and/or activity of integrin αᵥβ₃, disorders associated with abnormal bone metabolism, disorders associated with aberrant angiogenesis and cancers, or one or more conditions or symptoms associated therewith, said methods comprising administering to a subject in need thereof, one or more integrin αᵥβ₃ antagonists (e.g., one or more antibodies that immunospecifically bind to integrin αᵥβ₃, preferably VITAXIN® or an antigen-binding fragment thereof) in combination with one or more bisphosphonates and/or HMG-CoA reductase inhibitors optionally in further combination with one or more therapies (e.g., prophylactic or therapeutic agents) other than an integrin αᵥβ₃ antagonist, bisphosphonate or HMG-CoA reductase inhibitor. Therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules.

Any agent which is known to be useful, or which has been used or is currently being used for the prevention, treatment, management, or amelioration of an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or one or more conditions or symptoms associated therewith can be used in combination with an integrin αᵥβ₃ antagonist/HMG-CoA reductase inhibitor combination or an integrin αᵥβ₃ antagonist/bisphosphonate combination in accordance with the present invention. See, e.g., the Physician's Desk Reference (57^{th} ed., 2003) for information regarding prophylactic or therapeutic agents and other types of therapies which have been or are currently being used for treating, preventing, managing, or ameliorating inflammatory diseases, autoimmune disorders, disorders associated with aberrant expression and/or activity of integrin αᵥβ₃, disorders associated with abnormal bone metabolism, disorders associated with aberrant angiogenesis, cancer, or one or more symptoms or conditions associated with any of the foregoing. Examples of such agents and therapies include, but are not limited to, analgesics, anti-angiogenic agents, TNFα antagonists, anti-arthritics, antibiotics, anti-cancer therapies, anti-inflammatory agents, bone metabolism regulating agents, dental preparations, hormones, immunomodulatory agents, inhibitors of metalloproteinases, vitamins and minerals, chemotherapies, biological therapies, radiation therapies, and/or surgery.

4.5.1 Analgesics

Any analgesic well-known to one of skill in the art can be used in the compositions and the methods of the invention. Non-limiting examples of analgesics which can be used in accordance with the invention include: NSAIDs, salicylates, acetominophen, narcotics, and non-narcotic and anxiolytic combinations.

Analgesic agents and their dosages, routes of administration and recommended usage are known in the art and have been described in such literature as the Physician's Desk Reference (57^{th} ed., 2003).

4.5.2 Angioienesis Inhibitors

Angiogenesis inhibitors (i.e., anti-angiogenic agents) include, but are not limited to, angiostatin (plasminogen fragment), antiangiogenic antithrombin III, angiozyme, ABT-627, Bay 12-9566, Benefin, Bevacizumab, BMS-275291, cartilage-derived inhibitor (CDI), CAI, CD59 complement fragment, CEP-7055, Col 3, combretastatin A-4, endostatin (collagen XVIII fragment), fibronectin fragment, Gro-beta, Halofuginone, Heparinases, Heparin hexasaccharide fragment, HMV833, human chorionic gonadotropin (hCG), IM-862, Interferon alpha/beta/gamma, Interferon inducible protein (IP-10), Interleukin-12, Kringle 5 (plasminogen fragment), Marimastat, Metalloproteinase inhibitors (TIMPs) (see e.g., Section 4.5.12, infra), 2-methoxyestradiol, MMI 270 (CGS 27023A), MoAb IMC-1C11, Neovastat, NM-3, Panzem, PI-88, Placental ribonuclease inhibitor, plasminogen activator inhibitor, platelet factor-4 (PF4), Prinomastat, Prolactin 16 kD fragment, Proliferin-related protein (PRP), PTK 787/ZK 222594, retinoids, solimastat, squalamine, SS 3304, SU 5416, SU6668, SU11248, tetrahydrocortisol-S, tetrathiomolybdate, thalidomide, thrombospondin-1 (TSP-1), TNP-470, transforming growth factor-beta, vasculostatin, vasostatin (calreticulin fragment), ZD6126, and ZD 6474. In a specific embodiment, anti-angiogenic agents do not include antibodies or fragments thereof that immunospecifically bind to integrin α_{V}β₃.

Anti-angiogenesis agents and their dosages, routes of administration and recommended usage are known in the art and have been described in such literature as the Physician's Desk Reference (57^{th} ed., 2003).

4.5.3 TNF-α Antagonists

Any TNF-α antagonist well-known to one of skill in the art can be used in the compositions and methods of the invention. Non-limiting examples of TNF-α antagonists include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that block, reduce, inhibit or neutralize the function, activity and/or expression of TNF-α. In various embodiments, a TNF-α antagonist reduces the function, activity and/or expression of TNF-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS). Examples of antibodies that immunospecifically bind to TNF-α include, but are not limited to, infliximab (REMICADE™; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379). The present invention also encompasses the use of antibodies that immunospecifically bind to TNF-α disclosed in the following U.S. Patents in the compositions and methods of the invention: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety. Examples of soluble TNF-α receptors include, but are not limited to, sTNF-R1 (Amgen), etanercept (ENBREL™; Immunex) and its rat homolog RENBREL™, soluble inhibitors of TNF-α derived from TNFrI, TNFrII (Kohno et al., 1990, Proc. Natl. Acad. Sci. USA 87:8331-8335), and TNF-α Inh (Seckinger et al., 1990, Proc. Natl. Acad. Sci. USA 87:5188-5192).

Other TNF-α antagonists encompassed by the invention include, but are not limited to, IL-10, which is known to block TNF-α production via interferon γ-activated macrophages (Oswald et al., 1992, Proc. Natl. Acad. Sci. USA 89:8676-8680), TNFR-IgG (Ashkenazi et al., 1991, Proc. Natl. Acad. Sci. USA 88:10535-10539), the murine product TBP-1 (Serono/Yeda), the vaccine CytoTAb (Protherics), antisense molecule 104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SC10-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach.

TNF-α antagonists and their dosages, routes of administration and recommended usage are known in the art and have been described in such literature as the Physician's Desk Reference (57^{th} ed., 2003).

4.5.4 Anti-Arthritics

Any anti-arthritics well-known to one of skill in the art can be used in the compositions and the methods of the invention. Non-limiting examples of anti-arthritics which can be used in accordance with the invention include: analgesics (non-limiting examples are acetaminophen, in a dose up to 4000 mg/d; phenacetin; and tramadol, in a daily dose in the range of 200 to 300 mg); NSAIDs (non-limiting examples include but not limited to, aspirin, diflunisal, diclofenac, etodolac, fenamates, fenoprofen, flurbiprofen, ibuprofen, indomethacin, ketoprofen, methylsalicylate, nebumetone, naproxin, oxaprazin, phenylbutazone, piroxicam, sulindac, and tolmetin); nonacetylated salicylates such as salsalate; cyclooxygenase (Cox)-2-specific inhibitors (CSIs), such as, celecoxib and rofecoxib; intra- or periarticular injection of a depot glucocorticoid preparation; intra-articular injection of hyaluronic acid; capsaicin cream; copious irrigation of the osteroarthritis knee to flush out fibrin, cartilage shards and other debris; and joint replacement surgery. Low dose NSAIDs are preferred, e.g., ibuprofen at 1200 mg/d, naproxen at 500 mg/d. A gastroprotective agent, e.g., misoprostol, famotidine or omeprazole, is preferred to use concurrently with a NSAID).

Anti-arthritic agents and their dosages, routes of administration and recommended usage are known in the art and have been described in such literature as the Physician's Desk Reference (57^{th} ed., 2003).

In accordance with the invention, nonpharmacologic measures can be utilized in combination with an integrin αᵥβ₃ antagonist and an HMG-COA reductase inhibitor and/or a bisphosphonate for the prevention, management, or treatment of an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or one or more symptoms associated therewith. Examples of such non-pharmacological measures include, but are not limited to: reduction of joint loading (non-limiting examples are correction of poor posture, support for excessive lumbar lordosis, avoid excessive loading of the involved joint, avoid prolonged standing, kneeling and squatting); surgery (examples include but not limited to, arthroplasties, total joint replacement, reconstructive hand surgery, open or arthroscopic synovectomy, and early tenosynovectomy of the wrist); application of heat to the affected joint; aerobic exercise and other physical therapies.

4.5.5 Antibiotics

Any antibiotic well-known to one of skill in the art can be used in the compositions and the methods of the invention. Non-limiting examples of antibiotics include, penicillin, doxycycline, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

Antibiotic agents and their dosages, routes of administration and recommrnended usage are known in the art and have been described in such literature as the Physician's Desk Reference (57^{th} ed., 2003).

4.5.6 Anti-Cancer Therapies

Any therapy (e.g., chemotherapies, radiation therapies, hormonal therapies, and/or biological therapies/immunotherapies) which is known to be useful, or which has been used or is currently being used for the prevention, treatment, management or amelioration of cancer or one or more symptoms thereof can be used in accordance with the invention.

Specific examples of anti-cancer agents which can be used in accordance with the methods of the invention include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin-2 (including recombinant interieukin-2, or r1L2), interferon alpha-2a; interferon alpha-2b; interferon alpha-n1; interferon alpha-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vitaxin; vorozole; zanoterone; zeniplatin; zilascorb; and zinostatin stimalamer.

In specific embodiments, the anti-cancer agent is not an integrin αᵥβ₃ antagonist, an HMG CoA reductase inhibitor and/or a bisphosphonate.

The invention also encompasses use of radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radiaoactive source is placed inside the body close to cancer cells or a tumor mass.

In more particular embodiments, the present invention encompasses the use of one or more therapies such as, but not limited to anti-cancer agents such as those disclosed in Table 4, for the prevention, management, treatment or amelioration of cancer, preferably, breast, bone, ovary, and prostate cancers.

TABLE 4

| Therapeutic Agent | Dose/Administration/Formulation | | |
|---|---|---|---|
| doxorubicin | Intravenous | 60-75 mg/m² on Day 1 | 21 day intervals |
| hydrochloride | | | |
| (Adriamycin RDF ® | | | |
| and | | | |
| Adriamycin PFS ®) | | | |
| epirubicin | Intravenous | 100-120 mg/m² on Day | 3-4 week cycles |
| hydrochloride | | 1 of each cycle or | |
| (Ellence ™) | | divided equally and | |
| | | given on Days 1-8 of | |
| | | the cycle | |
| fluorouracil | Intravenous | How supplied: | |
| | | 5 mL and 10 mL vials | |
| | | (containing 250 and | |
| | | 500 mg flourouracil | |
| | | respectively) | |
| docetaxel | Intravenous | 60-100 mg/m² over 1 | Once every 3 weeks |
| (Taxotere ®) | | hour | |
| paclitaxel | Intravenous | 175 mg/m² over 3 hours | Every 3 weeks for |
| (Taxol ®) | | | 4 courses (administered |
| | | | sequentially to |
| | | | doxorubicin-containing |
| | | | combination |
| | | | chemotherapy) |
| tamoxifen citrate | Oral | 20-40 mg | Daily |
| (Nolvadex ®) | (tablet) | Dosages greater than 20 mg | |
| | | should be given in | |
| | | divided doses (morning | |
| | | and evening) | |
| leucovorin calcium for | Intravenous or | How supplied: | Dosage is unclear from |
| injection | intramuscular injection | 350 mg vial | text. PDR 3610 |
| Luprolide acetate | Single subcutaneous | 1 mg (0.2 mL or 20 unit | Once a day |
| (Lupron ®) | injection | mark) | |
| flutamide | Oral (capsule) | 250 mg | 3 times a day at 8 hour |
| (Eulexin ®) | | (capsules contain 125 mg | intervals (total daily |
| | | flutamide each) | dosage 750 mg) |
| nilutamide | Oral | 300 mg or 150 mg | 300 mg once a day for |
| (Nilandron ®) | (tablet) | (tablets contain 50 or | 30 days followed by |
| | | 150 mg nilutamide | 150 mg once a day |
| | | each) | |
| bicalutamide | Oral | 50 mg | Once a day |
| (Casodex ®) | (tablet) | (tablets contain 50 mg | |
| | | bicalutamide each) | |
| progesterone | Injection | USP in sesame oil 50 mg/mL | |
| ketoconazole | Cream | 2% cream applied once | |
| (Nizoral ®) | | or twice daily | |
| | | depending on | |
| | | symptoms | |
| prednisone | Oral | Initial dosage may vary | |
| | (tablet) | from 5 mg to 60 mg per | |
| | | day depending on the | |
| | | specific disease entity | |
| | | being treated. | |
| estramustine phosphate | Oral | 14 mg/kg of body | Daily given in 3 or 4 |
| sodium (Emcyt ®) | (capsule) | weight (i.e. one 140 mg | divided doses |
| | | capsule for each 10 kg | |
| | | or 22 lb of body | |
| | | weight) | |
| etoposide or | Intravenous | 5 mL of 20 mg/mL | |
| VP-16 | | solution (100 mg) | |
| dacarbazine | Intravenous | 2-4.5 mg/kg | Once a day for 10 days. |
| (DTIC-Dome ®) | | | May be repeated at 4 |
| | | | week intervals |
| polifeprosan 20 with | wafer placed in | 8 wafers, each | |
| carmustine implant | resection cavity | containing 7.7 mg of | |
| (BCNU) (nitrosourea) | | carmustine, for a total | |
| (Gliadel ®) | | of 61.6 mg, if size and | |
| | | shape of resection | |
| | | cavity allows | |
| Cisplatin | Injection | [n/a in PDR 861] | |
| | | How supplied: | |
| | | solution of 1 mg/mL in | |
| | | multi-dose vials of | |
| | | 50 mL and 100 mL | |
| mitomycin | Injection | supplied in 5 mg and 20 mg | |
| | | vials (containing 5 mg | |
| | | and 20 mg | |
| | | mitomycin) | |
| gemcitabine HCl | Intravenous | For NSCLC-2 | 4 week schedule- |
| (Gemzar ®) | | schedules have been | Days 1, 8 and 15 of each |
| | | investigated and the | 28-day cycle. Cisplatin |
| | | optimum schedule has | intravenously at 100 mg/m² |
| | | not been determined | on day 1 after |
| | | 4 week schedule- | the infusion of Gemzar. |
| | | administration | 3 week schedule- |
| | | intravenously at 1000 mg/m² | Days 1 and 8 of each 21 |
| | | over 30 minutes | day cycle. Cisplatin at |
| | | on 3 week schedule- | dosage of 100 mg/m² |
| | | Gemzar administered | administered |
| | | intravenously at 1250 mg/m² | intravenously after |
| | | over 30 minutes | administration of |
| | | | Gemzar on day 1. |
| carboplatin | Intravenous | Single agent therapy: | Every 4 weeks |
| (Paraplatin ®) | | 360 mg/m² I.V. on day 1 | |
| | | (infusion lasting 15 | |
| | | minutes or longer) | |
| | | Other dosage | |
| | | calculations: | |
| | | Combination therapy | |
| | | with | |
| | | cyclophosphamide, | |
| | | Dose adjustment | |
| | | recommendations, | |
| | | Formula dosing, etc. | |
| ifosamide | Intravenous | 1.2 g/m² daily | 5 consecutive days |
| (Ifex ®) | | | Repeat every 3 weeks |
| | | | or after recovery from |
| | | | hematologic toxicity |
| topotecan | Intravenous | 1.5 mg/m² by | 5 consecutive days, |
| hydrochloride | | intravenous infusion | starting on day 1 of 21 |
| (Hycamtin ®) | | over 30 minutes daily | day course |

Cancer therapies and their dosages, routes of administration and recommended usage are known in the art and have been described in such literature as the Physician's Desk Reference (57^{th} ed., 2003).

4.5.7 Anti-Inflammatory Agents

Any anti-inflammatory agents well-known to one of skill in the art can be used in the compositions and the methods of the invention. Non-limiting examples of anti-inflammatory agents which can be used in accordance with the invention to include: non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

Anti-inflammatory agents and their dosages, routes of administration and recommended usage are known in the art and have been described in such literature as the Physician's Desk Reference (57^{th} ed., 2003).

4.5.8 Bone Metabolism Regulating Agents

Any bone metabolism regulating therapy well-known to one of skill in the art can be used in the compositions and the methods of the invention. Non-limiting examples of bone metabolism regulating therapies include phosphate, aluminum hydroxide, aluminum carbonate gels, magnesium, vitamin D, calcitriol, vitamin D₂ (ergocalciferol), vitamin D₃ (cholecalciferol), calcium, lithium, glucocorticoids, calcitonin, plicamycin (mithramycin), gallium nitrate, estrogens, progestins, estrogen antagonists (e.g., tamoxifen), estrogen receptor modulators, androgen receptor modulators, cytotoxic or antiproliferative agents, matrix metalloproteinase inhibitors, inhibitors of epidermal-derived, fibroblast-derived, or platelet-derived growth factors, inhibitors of VEGF, antibodies to a growth factor or to a growth factor receptor, inhibitors of Flk-1/KDR, Flt-1, Tck/Tie-2, or Tie-1, cathepsin K inhibitors, inhibitors of osteoclast proton ATPase, inhibitors of urokinase plasminogen activator (u-PA), tumor-specific antibody-interleukin-2 fusion proteins, prenylation inhibitors, farnesyl transferase inhibitors, geranylgeranyl transferase inhibitors or dual farnesyl/geranylgeranyl transferase inhibitors, parathyroid hormone or parathyroid hormone fragments (a non-limiting example is exogenous PTH analogue, 1-34 PTH), growth hormones, molecules disclosed in U.S. Pat. Nos. 6,472,402 and 6,482,411, renal dialysis, surgery, or a combination thereof.

Examples of dosages, routes of administration and formulation of bone metbolism regulating agents which cab be used to treat, prevent, manage or ameliorate a bone metabolism disorder, or a symptom thereof are listed in Table 5, infra.

TABLE 5

| Therapeutic Agent | Dose/Administration/Formulation | | |
|---|---|---|---|
| raloxifene | Oral (tablet) | Each tablet contains 60 mg | Treatment or prevention |
| hydrochloride | | raloxifene HCl | of osteoporosis (60 mg |
| Evista ® | | (molar equivalent of | once daily, optionally in |
| (Eli Lilly) | | 55.71 mg of free base). | combination with 500 mg/day |
| | | | calcium and |
| | | | 400-600 IU/day vitamin D) |
| calcitonin | Injection | Sterile solution for | Treatment of Paget's |
| (calcitonin-salmon) | | subcutaneous or | disease (100 I.U. (5 mL) |
| Miacalcic ® (Novartis) | | intramuscular injection | injected per day, |
| | | (each mL contains 200 | monitored periodically) |
| | | I.U. calcitonin-salmon) | Treatment of |
| | | | hypercalcaeimia (4 I.U. |
| | | | injected subcutaneously |
| | | | or intramuscularly |
| | | | every 12 hours, may be |
| | | | increased to 8 I.U. |
| | | | every 12 hours if |
| | | | response is |
| | | | unsatisfactory after 1 or |
| | | | 2 days) |
| | | | Treatment of |
| | | | osteoporosis (100 I.U. |
| | | | injected subcutaneously |
| | | | or intramuscularly |
| | | | every other day, |
| | | | optionally in |
| | | | combination with 1.5 g |
| | | | calcium carbonate and |
| | | | 400 U vitamin D) |
| PDGF | | Under development by | Periodontal disease and |
| (e.g., recombinant | | ZymoGenetics, Inc. and | bone diseases |
| human platelet-derived | | BioMimetic | |
| growth factor) | | Pharmaceuticals, Inc. | |
| (Under development by | | | |
| ZymoGenetics, Inc. and | | | |
| BioMimetic | | | |
| Pharmaceuticals, Inc.) | | | |

Bone metabolism regulating agents and therapies and their dosages, routes of administration and recommended usage are known in the art and have been described in such literatures as the Physician's Desk Reference (57^{th} ed., 2003).

4.5.9 Dental Preparations

Any dental preparation well-known to one of skill in the art can be used in the compositions and the methods of the invention. Non-limiting examples of dental preparations that which can be used in accordance with the invention include fluoride, calcium, rinses (e.g., LISTERINE™), antibiotics (e.g., doxycycline hyclate a.k.a. PERIOSTAT™ (CollaGenex Pharmaceuticals, Inc., Newtown, Pa.), and Atridox™ (Atrix Laboratories, Inc.), fluoride and calcium supplements (e.g., FLORICAL™ (Mericon Industries, Inc., Peoria, Ill.) and MONOCAL™ (Mericon Industries, Inc., Peoria, Ill.)).

In addition, periodontal vaccines can also be used in accordance with the invention. See DeCarlo, A., et al., 2003, Infect. Immun. 71(1):562-6 (perinoculation with Porphyromonas gingival HA2 binding domain for hemoglobin found to provide protection from periodontitis in rat periodontitis model); and Rajapakse, P. et al., 2002, Infection and Immunity 70(5):2480-2486 (immunization with RgpA-Kgp proteinase-adhesion complexes of Porphyromonas gingivalis found to protect against periodontal bone loss in rat periodontitis model), incorporated by reference in their entireties.

Dental preparations and their dosages, routes of administration and recommended usage are known in the art and have been described in such literature as the Physician's Desk Reference (57^{th} ed., 2003).

4.5.10 Hormones

Any hormone well-known to one of skill in the art can be used in the compositions and the methods of the invention. Non-limiting examples of hormones which can be used in the compositions and methods of the invention include calcitonin, glucocorticoids, progestins, androgens, and estrogen.

Homones and their dosages, routes of administration and recommended usage are known in the art and have been described in such literatures as the Physician's Desk Reference (57^{th} ed., 2003).

4.5.11 Immunomodulatory Agents

Any immunomodulatory agent well-known to one of skill in the art can be used in the compositions and the methods of the invention. Non-limiting examples of immunomodulatory agents which can be used in used in accordance with the invention include chemotherapeutic agents and immunomodulatory agents other than chemotherapeutic agents. Examples of chemotherapeutic agents include, but are not limited to: methotrexate, cyclosporin A, malononitriloamindes (e.g., leflunamide), cisplatin, ifosfamide, taxanes such as paclitaxol or docetaxel, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, Immuran, minocycline, azathioprine, antibiotics (e.g., FK506 (tacrolimus)), methylprednisolone (MP), corticosteroids, steriods, mycophenolate mofetil, rapamycin (sirolimus), mizoribine, deoxyspergualin, brequinar, glucocorticoids, procaine, tetracaine, lidocaine, propranolol, and puromycin homologs, and cytoxan.

Examples of immunomodulatory agents other than chemotherapeutic agents include, but are not limited to: anti-immune receptor antibodies (such as anti-T cell receptor antibodies, e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., MEDI-507 (MedImmune, Inc.; International Publication Nos. WO 02/098370 and WO 02/069904), anti-CD 11a antibodies (e.g., Xanelim (Genentech)), and anti-B7 antibodies (e.g., IDEC-114) (IDEC)); anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, anti-IL-12 receptor antibodies, and anti-IL-23 receptor antibodies); soluble immune cell receptors (e.g., CTLA4-immunoglobulin and LFA-3TIP (Biogen, International Publication No. WO 93/08656 and U.S. Pat. No. 6,162,432)); soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof); cytokines or fragments thereof (e.g., interleukin (IL)-2, IL-3, IL-4, IL-5, IL-6, IL-7, IL-8, IL-9, IL-10, IL-11, IL-12, IL-15, IL-23, TNF-α, TNF-β, interferon (IFN)-α, IFN-β, IFN-γ, and GM-CSF); and anti-cytokine antibodies (e.g., anti-IL-2 antibodies, anti-IL-4 antibodies, anti-IL-6 antibodies, anti-IL-8 antibodies (e.g., ABX-IL-8 (Abgenix)), anti-IL-9 antibodies, anti-IL-10 antibodies, anti-IL-12 antibodies, anti-IL-15 antibodies, anti-IL-23 antibodies, anti-TNF-α antibodies, and anti-IFN-γ antibodies).

Immunomodulatory agents and their dosages, routes of administration and recommended usage are known in the art and have been described in such literatures as the Physician's Desk Reference (57^{th} ed., 2003).

4.5.12 Inhibitors of Metalloproteinases

Any inhibitor of a metalloproteinase(s) well-known to one of skill in the art can be used in the compositions and the methods of the invention. Non-limiting examples of inhibitors of metalloproteinases include: Marimastat, BB94 (batimastat; [4-(N-hydroxyamino)-2R-isobutyl-3S-thienyl-thiomethyl)-succinyl-]L-phenylalanine-N-methylamide; British Pharmaceuticals Limited (Oxford, UK)), Streptomyces metalloproteinase inhibitor (SMPI), BB-3103, and tissue inhibitors of metalloproteinases (e.g., TIMP-1, TIMP-2 and TIMP-3).

Inhibitors of metalloproteinases and their dosages, routes of administration and recommended usage are known in the art and have been described in such literature as the Physician's Desk Reference (57^{th} ed., 2003).

4.5.13 Vitamins and Minerals

Any vitamin and/or mineral well-known to one of skill in the art can be used in the compositions and the methods of the invention. Non-limiting examples of vitamins and minerals include magnesium, zinc, calcium, vitamin C compounds, vitamin B compounds such as vitamin B3, vitamin B6 (pyridoxine), vitamin B12, vitamin D compounds, vitamin E, vitamin A, iron, Selenium, folic acid and biotin. Preferably, a multivitamin and a mineral supplement is used in accordance with the invention.

Vitamins and minerals and their dosages, routes of administration and recommended usage are known in the art and have been described in such literatures as the Physician 's Desk Reference (57^{th} ed., 2003).

4.6 Prophylactic & Therapeutic Uses for the Combination Therapies of the Invention

The present invention is directed to combination therapies which involve administering one or more integrin αᵥβ₃ antagonists (preferably, antibodies that immunospecifically bind to integrin αᵥβ₃) and compositions comprising said antagonists to a subject, preferably a human subject, in combination with one or more HMG Co-A reductase inhibitors and/or one or bisphosphonates, and optionally one or more other therapies (e.g., prophylactic or therapeutic agents) for the prevention, treatment, management or amelioration of a disease or disorder or one or more symptoms thereof. The invention provides methods of preventing, treating, managing, or ameliorating a disease or disorder or one or more symptoms thereof, said methods comprising administering to a subject in need thereof one or more integrin αᵥβ₃ antagonists, one or more HMG Co-A reductase inhibitors, and optionally, one or more other therapies that are currently being used, have been used, or are known to be useful in the prevention, treatment or amelioration of said disease or disorder or one or more symptoms thereof. The invention also provides methods of preventing, treating, managing or ameliorating a disease or disorder or one or more symptoms thereof, said methods comprising administering to a subject in need thereof one or more integrin αᵥβ₃ antagonists, one or more bisphosphonates, and optionally, one or more other therapies that are currently being used, have been used or are known to be useful in the prevention, management, treatment of said disease or disorder or one or more symptoms thereof. The invention further provides methods of preventing, treating, managing or ameliorating a disease or disorder or one or more symptoms thereof comprising administering to a subject in need thereof one or more integrin αᵥβ₃ antagonists, one or more bisphosphonates, one or more HMG Co-A reductase inhibitors, and optionally, one or more other therapies that are currently being used, have been used or are known to be useful in the prevention, management, treatment of said disease or disorder or one or more symptoms thereof.

The prophylactic or therapeutic agents of the combination therapies of the invention can be administered sequentially or concurrently. In a specific embodiment, the combination therapies of the invention comprise one or more integrin αᵥβ₃ antagonists, one or more HMG Co-A reductase inhibitors and/or one or more bisphosphonates, and at least one other prophylactic or therapeutic agent which has a different mechanism of action than said antagonists HMG Co-A reductase inhibitors and/or bisphosphonates. In certain embodiments, the combination therapies of the present invention improve the prophylactic or therapeutic effect of one or more integrin αᵥβ₃ antagonists, by functioning together with the antagonists to have an additive or synergistic effect. In certain embodiments, the combination therapies of the present invention reduce the side effects associated with the prophylactic or therapeutic agents.

The prophylactic or therapeutic agents of the combination therapies can be administered to a subject, preferably a human subject, in the same pharmaceutical composition. In alternative embodiments, the prophylactic or therapeutic agents of the combination therapies can be administered concurrently to a subject in separate pharmaceutical compositions. The prophylactic or therapeutic agents may be administered to a subject by the same or different routes of administration.

Diseases and disorders which can be prevented, treated, managed, or ameliorated by administering an effective amount of one or more antibodies of the invention include, but are not limited to, osteoporosis, Gorham-Stout disease, prostate cancer, breast cancer, arthritis, and the diseases listed in Sections 4.6.1 through 4.6.5. In a preferred embodiment, the combination therapies are administered to a subject, preferably a human subject, according to the methods of the invention to prevent, treat, manage, or ameliorate one or more symptoms of a disease or disorder associated with aberrant bone metabolism.

4.6.1 Treatment or Prevention of Disorders Associated with Inflammation

One or more integrin αᵥβ₃ antagonists or compositions comprising said antagonists in combination with one or more HMG Co-A reductase inhibitors and/or one or more bisphosphonates may be administered to a subject in need thereof to prevent, manage, treat or ameliorate an inflammatory disorder (i.e., a disorder associated with or characterized by inflammation of one or more joints, tissues or organs) or one or more symptoms thereof One or more integrin αᵥβ₃ antagonists or compositions comprising said antagonists may also be administered in combination with one or more HMG Co-A reductase inhibitors and/or one or more bisphosphonates and one or more other therapies useful for the prevention, management, treatment or amelioration of an inflammatory disorder. Non-limiting examples of such therapies include those disclosed in Section 4.5 supra, in particular the analgesics, TNF-α antagonists, anti-arthritics, anti-inflammatory agents, dental preparations, and immunomodulatory agents, disclosed in Sections 4.5.1, 4.5.3, 4.5.4, 4.5.6, 4.5.7, 4.5.9, and 4.5.11 supra) to a subject in need thereof to prevent, manage, treat or ameliorate an inflammatory disorder or one or more symptoms thereof.

In a specific embodiment, the invention provides a method of preventing, managing, treating or ameliorating an inflammatory disorder or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, and optionally, a dose of a prophylactically or therapeutically effective amount of one or more therapies other than integrin αᵥβ₃ antagonists and HMG Co-A reductase inhibitors. In another embodiment, the invention provides a method of preventing, managing, treating or ameliorating an inflammatory disorder or one or more symptoms thereof, said method comprising administering to a subject in need thereof a dose of a prophylactically or therapeutically effective amount of one or more integrin αᵥβ₃ antagonists, a dose of prophylactically or therapeutically effective amount of one or more bisphosphonates, and optionally, a dose of a prophylactically or therapeutically effective amount of one or more therapies other than integrin αᵥβ₃ antagonists and bisphosphonates. In another embodiment, the invention provides a method of preventing, managing, treating or ameliorating an inflammatory disorder or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, a dose of a prophylactically or therapeutically effective amount of one or more bisphosphonates, and optionally, a dose of a prophylactically or therapeutically effective amount of one or more therapies other than an integrin αᵥβ₃ antagonist, an HMG Co-A reductase inhibitor and/or a bisphosphonate.

The combination therapies of the invention may be used as a first, second, third, fourth or fifth line of treatment. The invention provides methods for managing, treating or ameliorating one or more symptoms of an inflammatory disorder in a subject refractory to conventional therapies (e.g., methotrexate and a TNF-α antagonist (e.g., REMICADE™ or ENBREL™)) for such an inflammatory disorder, said methods comprising administering to said subject a dose of a prophylactically or therapeutically effective amount of one or more integrin αᵥβ₃ antagonists and a dose of a prophylactically or therapeutically effective amount of one or more HMG Co-A reductase inhibitors and/or a dose of a prophylactically or therapeutically effective amount of one or more bisphosphonates, and optionally, a dose of a prophylactically or therapeutically effective amount of one or more therapies other than an integrin αᵥβ₃ antagonists and an HMG Co-A reductase inhibitor and/or a bisphosphonate.

The invention also provides methods for preventing, managing, treating or ameliorating an inflammatory disorder or one or more symptoms thereof in a subject refractory to existing single agent therapies for such an inflammatory disorder, said methods comprising administering to said subject a dose of an effective amount of one or more integrin αᵥβ₃ antagonists, a dose of an effective amount of one or more HMG Co-A reductase inhibitors and optionally a dose of an effective amount of one or more therapies other than an integrin αᵥβ₃ antagonist, and an HMG Co-A reductase inhibitor.

The invention also provides methods for managing, treating or ameliorating an inflammatory disorder or one or more symptoms thereof in a subject refractory to existing single agent therapies, said methods comprising administering to said subject a dose of an effective amount of one or more integrin αᵥβ₃ antagonists, a dose of an effective amount of one or more bisphosphonates, and optionally, a dose of an effective amount of one or more therapies other than an integrin αᵥβ₃ antagonist and a bisphosphonate. The invention also provides methods for managing or treating an inflammatory disorder by administering one or more integrin αᵥβ₃ antagonists and one or more HMG Co-A reductase inhibitors and/or one or more bisphosphonates in combination with any other therapy to patients who have proven refractory to other therapies but are no longer on these therapies. The invention also provides alternative methods for the treatment of an inflammatory disorder where another therapy has proven or may prove too toxic, i.e., results in unacceptable or unbearable side effects, for the subject being treated. For example, one or more integrin αᵥβ₃ antagonists and one or more HMG Co-A reductase inhibitors and/or one or more bisphosphonates may be administered to a subject, wherein the subject is refractory to a TNF-α antagonist or methotrexate. Further, the invention provides methods for preventing the recurrence of an inflammatory disorder in patients that have been treated and have no disease activity by administering one or more integrin αᵥβ₃ antagonists and one or more HMG Co-A reductase inhibitors and/or one or more bisphosphonates.

Inflammatory disorders that can be treated by the methods encompassed by the invention include, but are not limited to, asthma, Behcet's disease, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, osteoarthritis, spondyloarthropathies (e.g., psoriatic arthritis, ankylosing spondylitis, Reiter's Syndrome (reactive arthritis), inflammatory osteolysis, Wilson's disease and chronic inflammation resulting from chronic viral or bacteria infections.

In specific embodiments, patients with osteoarthritis are administered a prophylactically or therapeutically effective amount of one or more integrin αᵥβ₃ antagonists and one or more HMG Co-A reductase inhibitors and/or one or more bisphosphonates in combination with therapies useful for osteoarthritis prevention, treatment, management or amelioration, other than an integrin αᵥβ₃ antagonist, an HMG Co-A reductase inhibitor, and/or a bisphosphonate. Examples of such therapies include, but are not limited to: copious irrigation of the osteroarthritis knee to flush out fibrin, cartilage shards and other debris; and joint replacement surgery, and any of the agents listed in Section 4.5, in particular, analgesics (non-limiting examples are acetaminophen, in a dose up to 4000 mg/d; phenacetin; and tramadol, in a daily dose in the range of 200 to 300 mg); NSAIDs (non-limiting examples include but not limited to, aspirin, diflunisal, diclofenac, etodolac, fenamates, fenoprofen, flurbiprofen, ibuprofen, indomethacin, ketoprofen, methylsalicylate, nebumetone, naproxen, naproxen sodium, oxaprazin, phenylbutazone, piroxicam, sulindac, and tolmetin); cyclooxygenase-2-specific inhibitors (COX-2 inhibitors (CSIs), e.g., rofecoxib (Vioxx®) and celecoxib (Celebrex®)); nonacetylated salicylates including but not limited to salsalate; intra- or periarticular injection of a depot glucocorticoid preparation; intra-articular injection of a depot glucocorticoid preparation; intra-articular injection of hyaluronic acid; and capsaicin cream. Low dose NSAIDs are preferred, e.g., ibuprofen at 1200 mg/d, naproxen at 500 mg/d. A gastroprotective agent, e.g., misoprostol, famotidine or omeprazole, is preferable used concurrently with a NSAID). Nonpharmacologic measures can also be used in conjunction with the combination therapies of the invention in the prevention, treatment, management and amelioration of osteoarthritis. Examples of nonpharmacologic measures include, but are not limited to, reduction of joint loading (non-limiting examples are correction of poor posture, support for excessive lumbar lordosis, avoid excessive loading of the involved joint, avoid prolonged standing, kneeling and squatting); application of heat to the affected joint; and aerobic exercise and other physical therapies.

In specific embodiments, patients with rheumatoid arthritis are administered a prophylactically or therapeutically effective amount of one or more integrin αᵥβ₃ antagonists and a prophylactically or therapeutically effective amount of one or more bisphosphonates and/or a prophylactically or therapeutically effective amount of one or more HMG Co-A reductase inhibitors in combination with other therapies useful in prevention, treatment, management and amelioration of rheumatoid arthritis, (see, e.g., Section 5.5, supra). Nonlimiting examples of such therapies include NSAIDs (non-limiting examples include aspirin, diflunisal, diclofenac, etodolac, fenamates, fenoprofen, flurbiprofen, ibuprofen, indomethacin, ketoprofen, methylsalicylate, nebumetone, naproxin, oxaprazin, phenylbutazone, piroxicam, sulindac, and tolmetin.); analgesics (non-limiting examples are acetaminophen, phenacetin and tramadol); CSIs including but not limited to, celecoxib and rofecoxib; glucocorticoids (preferably low-dose oral glucocorticoids, e.g., <7.5 mg/d prednisone, or monthly pulses with high-dose glucocorticoids, or intraarticular glucocorticoids); disease-modifying antirheumatic drugs (DMARDs) including but not limited to, methotrexate (preferably given intermittent low dose, e.g., 7.5-30 mg once weekly), gold compounds (e.g., gold salts), D-penicillamine, the antimalarials (e.g., chloroquine), and sulfasalazine; TNF-α antagonist agents including but not limited to, etanercept and infliximab; immunosuppressive and cytotoxic agents (examples include but not limited to, azathioprine, leflunomide, cyclosporine, and cyclophosphamide), and surgery (examples include but not limited to, arthroplasties, total joint replacement, reconstructive hand surgery, open or arthroscopic synovectomy, and early tenosynovectomy of the wrist). One or more integrin αᵥβ₃ antagonists and one or more HMG Co-A reductase inhibitors and/or one or more bisphosphonates may also be used in combination with other measures in prevention, treatment, management and amelioration of the rheumatoid arthritis including but not limited to: rest, splinting to reduce unwanted motion of inflamed joint, exercise, used of a variety of orthotic and assistive devices, and other physical therapies. One or more integrin αᵥ β₃ antagonists and one or more HMG Co-A reductase inhibitors and/or one or more bisphosphonates may also be used in combination with some nontraditional approaches in prevention, treatment, management and amelioration of rheumatoid arthritis including but not limited to, diets (e.g., substituting omega-3 fatty acids such as eicosapentaenoic acid found in certain fish oils for dietary omega-6 essentail fatty acids found in meat), vaccines, hormones and topical preparations.

Anti-inflammatory therapies and their dosages, routes of administration and recommended usage are known in the art and have been described in such literature as the Physician 's Desk Reference (57^{th} ed., 2003).

4.6.2 Treatment or Prevention of Autoimmune Disorders

One or more integrin αᵥβ₃ antagonists or compositions comprising said antagonists in combination with one or more HMG Co-A reductase inhibitors and/or one or more bisphosphonates may be administered to a subject in need thereof to prevent, manage, treat or ameliorate an autoimmune disorder or one or more symptoms thereof. One or more integrin αᵥβ₃ antagonists or compositions comprising said antagonists may also be administered in combination with one or more HMG Co-A reductase inhibitors and/or one or more bisphosphonates and one or more other therapies useful for the prevention, management, treatment or amelioration of an autoimmune disorder. Non-limiting examples of such therapies include those disclosed in Section 4.5 supra, in particular analgesics, anti-inflammatory agents, and immunomodulatory agents such as those disclosed in Sections 4.5.1, 4.5.7, and 4.5.11 supra) to a subject in need thereof to prevent, manage, treat or ameliorate an autoimmune disorder or one or more symptoms thereof.

In a specific embodiment, the invention provides a method of preventing, managing, treating or ameliorating an autoimmune disorder or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, and optionally, a dose of a prophylactically or therapeutically effective amount of one or more therapies other than integrin αᵥβ₃ antagonists and HMG Co-A reductase inhibitors. In another embodiment, the invention provides a method of preventing, managing, treating or ameliorating an autoimmune disorder or one or more symptoms thereof, said method comprising administering to a subject in need thereof a dose of a prophylactically or therapeutically effective amount of one or more integrin αᵥβ₃ antagonists, a dose of prophylactically or therapeutically effective amount of one or more bisphosphonates, and optionally, a dose of a prophylactically or therapeutically effective amount of one or more therapies other than integrin αᵥβ₃ antagonists and bisphosphonates. In another embodiment, the invention provides a method of preventing, managing, treating or ameliorating an autoimmune disorder or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, a dose of a prophylactically or therapeutically effective amount of one or more bisphosphonates, and optionally, a dose of a prophylactically or therapeutically effective amount of one or more therapies other than an integrin αᵥβ₃ antagonist, an HMG Co-A reductase inhibitor and/or a bisphosphonate.

The combination therapies of the invention may be used as a first, second, third, fourth or fifth line of treatment. The invention provides methods for managing, treating or ameliorating one or more symptoms of an autoimmune disorder in a subject refractory to conventional therapies for such an autoimmune disorder (e.g., a steroid), said methods comprising administering to said subject a dose of a prophylactically or therapeutically effective amount of one or more integrin αᵥβ₃ antagonists and a dose of a prophylactically or therapeutically effective amount of one or more HMG Co-A reductase inhibitors and/or a dose of a prophylactically or therapeutically effective amount of one or more bisphosphonates, and optionally, a dose of a prophylactically or therapeutically effective amount of one or more therapies other than an integrin αᵥβ₃ antagonists and an HMG Co-A reductase inhibitor and/or a bisphosphonate.

The invention also provides methods for preventing, managing, treating or ameliorating an autoimmune disorder or one or more symptoms thereof in a subject refractory to existing single agent therapies for such an autoimmune disorder, said methods comprising administering to said subject a dose of an effective amount of one or more integrin αᵥβ₃ antagonists, a dose of an effective amount of one or more HMG Co-A reductase inhibitors and optionally a dose of an effective amount of one or more therapies other than an integrin αᵥβ₃ antagonist and an HMG Co-A reductase inhibitor. The invention also provides methods for managing, treating or ameliorating an autoimmune disorder or one or more symptoms thereof in a subject refractory to existing single agent therapies, said methods comprising administering to said subject a dose of an effective amount of one or more integrin αᵥβ₃ antagonists, a dose of an effective amount of one or more bisphosphonates, and optionally, a dose of an effective amount of one or more therapies, other than an integrin αᵥβ₃ antagonist and a bisphosphonate. The invention also provides methods for managing or treating an autoimmune disorder by administering one or more integrin αᵥβ₃ antagonists and one or more HMG Co-A reductase inhibitors and/or one or more bisphosphonates in combination with any other therapy to patients who have proven refractory to other therapies but are no longer on these therapies. The invention also provides alternative methods for the treatment of an autoimmune disorder where another therapy has proven or may prove too toxic, i.e., results in unacceptable or unbearable side effects, for the subject being treated. For example, one or more integrin αᵥβ₃ antagonists and one or more HMG Co-A reductase inhibitors and/or one or more bisphosphonates may be administered to a subject, wherein the subject is refractory to a steroid. Further, the invention provides methods for preventing the recurrence of an autoimmune disorder in patients that have been treated and have no disease activity by administering one or more integrin αᵥβ₃ antagonists and one or more HMG Co-A reductase inhibitors and/or one or more bisphosphonates.

In autoimmune disorders, the immune system triggers an immune response when there are no foreign substances to fight and the body's normally protective immune system causes damage to its own tissues by mistakenly attacking self. There are many different autoimmune disorders which affect the body in different ways. For example, the brain is affected in individuals with multiple sclerosis, the gut is affected in individuals with Crohn's disease, and the synovium, bone and cartilage of various joints are affected in individuals with rheumatoid arthritis. As autoimmune disorders progress destruction of one or more types of body tissues, abnormal growth of an organ, or changes in organ function may result. The autoimmune disorder may affect only one organ or tissue type or may affect multiple organs and tissues. Organs and tissues commonly affected by autoimmune disorders include red blood cells, blood vessels, connective tissues, endocrine glands (e.g., the thyroid or pancreas), muscles, joints, and skin. Examples of autoimmune disorders that can be treated by the methods of the invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dennatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis.

Autoimmune therapies and their dosages, routes of administration and recommended usage are known in the art and have been described in such literature as the Physician's Desk Reference (57^{th} ed., 2003).

4.6.3 Treatment or Prevention of Disorders Associated with Aberrant Bone Metabolism

One or more integrin αᵥβ₃ antagonists or compositions comprising said antagonists in combination with one or more HMG Co-A reductase inhibitors and/or one or more bisphosphonates may be administered to a subject in need thereof to prevent, manage, treat or ameliorate a disorder associated with aberrant bone metabolism or one or more symptoms thereof. One or more integrin UV,3 antagonists or compositions comprising said antagonists may also be administered in combination with one or more HMG Co-A reductase inhibitors and/or one or more bisphosphonates and one or more other therapies useful for the prevention, management, treatment or amelioration of a disorder associated with aberrant bone metabolism. Non-limiting examples of such therapies include those discussed in Section 4.5 supra, in particular, analgesics, anti-arthritics, antibiotics, anti-cancer agents, anti-inflammatory agents, bone metabolism regulating agents, dental preparations and hormones such as those disclosed in Section 4.5 supra, in particular the agents disclosed in Sections 4.5.1, 4.5.4, 4.5.5, 4.5.6, 4.5.7, 4.5.8, 4.5.9, 4.5.10, and 4.5.13 supra) to a subject in need thereof to prevent, manage, treat or ameliorate a disorder associated with aberrant bone metabolism or one or more symptoms thereof.

In a specific embodiment, the invention provides a method of preventing, managing, treating or ameliorating a disorder associated with aberrant bone metabolism or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, and optionally, a dose of a prophylactically or therapeutically effective amount of one or more therapies other than integrin αᵥβ₃ antagonists and HMG Co-A reductase inhibitors. In another embodiment, the invention provides a method of preventing, managing, treating or ameliorating a disorder associated with aberrant bone metabolism or one or more symptoms thereof, said method comprising administering to a subject in need thereof a dose of a prophylactically or therapeutically effective amount of one or more integrin αᵥβ₃ antagonists, a dose of prophylactically or therapeutically effective amount of one or more bisphosphonates, and optionally, a dose of a prophylactically or therapeutically effective amount of one or more therapies other than integrin αᵥβ₃ antagonists and bisphosphonates. In another embodiment, the invention provides a method of preventing, managing, treating or ameliorating a disorder associated with aberrant bone metabolism or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, a dose of a prophylactically or therapeutically effective amount of one or more bisphosphonates, and optionally, a dose of a prophylactically or therapeutically effective amount of one or more therapies other than an integrin αᵥβ₃ antagonist, an HMG Co-A reductase inhibitor and/or a bisphosphonate.

The combination therapies of the invention may be used as a first, second, third, fourth or fifth line of treatment. The invention provides methods for managing, treating or ameliorating a disorder associated with aberrant bone metabolism or one or more symptoms thereof in a subject refractory to conventional therapies for such a disorder associated with aberrant bone metabolism (e.g., estrogen or progestin), said methods comprising administering to said subject a dose of a prophylactically or therapeutically effective amount of one or more integrin αᵥβ₃ antagonists and a dose of a prophylactically or therapeutically effective amount of one or more HMG Co-A reductase inhibitors and/or a dose of a prophylactically or therapeutically effective amount of one or more bisphosphonates, and optionally, a dose of a prophylactically or therapeutically effective amount of one or more therapies other than an integrin αᵥβ₃ antagonists and an HMG Co-A reductase inhibitor and/or a bisphosphonate.

The invention also provides methods for preventing, managing, treating or ameliorating a disorder associated with aberrant bone metabolism or one or more symptoms thereof in a subject refractory to existing single agent therapies for such a disorder associated with aberrant bone metabolism, said methods comprising administering to said subject a dose of a prophylactically or therapeutically effective amount of one or more integrin αᵥβ₃ antagonists, a dose of an effective amount of one or more HMG Co-A reductase inhibitors other than an integrin αᵥβ₃ antagonist and a bisphosphonate, and optionally a dose of an effective amount of one or more therapies other than an integrin αᵥβ₃ antagonist, and an HMG Co-A reductase inhibitor. The invention also provides methods for managing, treating or ameliorating a disorder associated with aberrant bone metabolism or one or more symptoms thereof in a subject refractory to existing single agent therapies, said methods comprising administering to said subject a dose of an effective amount of one or more integrin αᵥβ₃ antagonists, a dose of an effective amount of one or more bisphosphonates, and optionally, a dose of an effective amount of one or more therapies. The invention also provides methods for managing or treating a disorder associated with aberrant bone metabolism by administering one or more integrin αᵥβ₃ antagonists and one or more HMG Co-A reductase inhibitors and/or one or more bisphosphonates in combination with any other therapy to patients who have proven refractory to other therapies but are no longer on these therapies. The invention also provides alternative methods for the treatment of a disorder associated with aberrant bone metabolism where another therapy has proven or may prove too toxic, i.e., results in unacceptable or unbearable side effects, for the subject being treated. For example, one or more integrin αᵥβ₃ antagonists and one or more HMG Co-A reductase inhibitors and/or one or more bisphosphonates may be administered to a subject, wherein the subject is refractory to estrogen therapy. Further, the invention provides methods for preventing the recurrence of a disorder associated with aberrant bone metabolism in patients that have been treated and have no disease activity by administering one or more integrin αᵥβ₃ antagonists and one or more HMG Co-A reductase inhibitors and/or one or more bisphosphonates.

Diseases or disorders that are associated with or characterized by aberrant bone metabolism include, but not limited to, osteoporosis (including, but not limited to, post-menopausal osteoporosis and inflammation-induced osteoporosis); rickets and osteomalacia; hypercalcemia which can be caused by, but not limited to, primary hyperparathyroidism (e.g., solitary adenomas and multiple endocrine neoplasia), hereditary hypoparathyroidism, acquired hypoparathyroidism, chronic renal failure, vitamin D deficiency, tumor lysis, rhabdomyolysis, and osteitis fibrosa after parathyroidectomy; lithium therapy; familial hypocalciuric hypercalcemia; solid tumors with metastases (e.g., breast cancer); solid tumors with humoral mediation of hypercalcemia (e.g., lung or kidney cancer); hematologic malignancies (e.g., multiple myeloma, lymphoma, leukemia); vitamin D intoxication; sarcoidosis and other granulomatous diseases; idopathic hypercalcemia of infancy; hyperthyroidism; vitamin A intoxication; aluminum intoxication; milk-alkali syndrome; renal failure; diseases associated with an increased risk of generalized osteoporosis in adults including but not limited to, Turner syndrome, Klinefelter syndrome, anorexia nervosa, hypothalamic amenorrhea, hyperprolactinemia, primary or secondary hypogonadal state, Cushing's syndrome, hyperparathyroidism, thyrotoxicosis, insulin-dependent diabetes mellitus, acromegaly, adrenal insufficiency, malnutrition, parenteral nutrition, malabsorption syndromes, gastrectomy, severe liver disease, pernicious anemia; rheumatoid arthritis; ankylosing spondylitis; cholesteatoma-induced bone resorption; hypertrophic pulmonary osteoarthropathy (HPOA); Gorham-Stout disease; multiple myloma; lymphoma and leukemia; malignancy-associated parathyroid hormone related production; mastocytosis; osteogenesis imperfecta; Marfan syndrome; hemochromatosis; hypophosphatasia; glycogen storage diseases; homocystinuria; Ehlers-Danlos syndrome; porphyria; Menkes' syndrome; epidermolysis bullosa; chronic obstructive pulmonary disease; Scoliosis; multiple sclerosis; sacoidosis and amyloidosis; drug related osteoporosis, which can be caused by, but not limited to, glucocorticoids, cyclosporine, cytotoxic drugs, anticonvulsants, excessive thyroxine, aluminum, gonadotropin-releasing hormone agonists, heparin and lithium; Paget's disease of bone; osteopetrosis (Albers-Schonberg bone disease); pyknodysostosis; osteomyelosclerosis; hereditary hyperphosphatasia; progressive diaphyseal dysplasia (Camurati-Engelmann disease); melorheostosis; osteopoikilosis; hyperostosis frontalis interna; fibrous dysplasia (Mccune-Albright syndrome); spndyloepiphyseal dysplasia; achondroplasia; enchondromatosis; ostoechondromatosis; aseptic loosening of a joint; and periodontal disease.

One or more integrin αᵥβ₃ antagonists or compositions comprising said antagonists in combination with one or more HMG Co-A reductase inhibitors and/or one or more bisphosphonates may be administered to a subject in need thereof to prevent, manage, treat or ameliorate a disorder associated with abnormal bone resorption or one or more symptoms thereof. One or more integrin αᵥβ₃ antagonists or compositions comprising said antagonists may also be administered in combination with one or more HMG Co-A reductase inhibitors and/or one or more bisphosphonates and one or more other therapies useful for the prevention, management, treatment or amelioration of a disorder associated with abnormal bone resorption to a subject in need thereof to prevent, manage, treat or ameliorate a disorder associated with abnormal bone resorption. Non-limiting examples of such therapies include analgesics, anti-arthritics, anti-inflammatory agents, bone metabolism regulating agents, dental preparations and hormones such as those disclosed in Section 4.5 supra, in particular the agents disclosed in Sections 4.5.1, 4.5.4, 4.5.7, 4.5.8, 4.5.9, and 4.5.10 supra) to a subject in need thereof to prevent, manage, treat or ameliorate a disorder associated with abnormal bone resorption or one or more symptoms thereof.

In a specific embodiment, the invention provides a method of preventing, managing, treating or ameliorating a disorder associated with abnormal bone resorption or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, and optionally, a dose of a prophylactically or therapeutically effective amount of one or more therapies other than integrin αᵥβ₃ antagonists and HMG Co-A reductase inhibitors. In another embodiment, the invention provides a method of preventing, managing, treating or ameliorating a disorder associated with abnormal bone resorption or one or more symptoms thereof, said method comprising administering to a subject in need thereof a dose of a prophylactically or therapeutically effective amount of one or more integrin αᵥβ₃ antagonists, a dose of prophylactically or therapeutically effective amount of one or more bisphosphonates, and optionally, a dose of a prophylactically or therapeutically effective amount of one or more therapies other than integrin αᵥβ₃ antagonists and bisphosphonates. In another embodiment, the invention provides a method of preventing, managing, treating or ameliorating a disorder associated with abnormal bone resorption or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, a dose of a prophylactically or therapeutically effective amount of one or more bisphosphonates, and optionally, a dose of a prophylactically or therapeutically effective amount of one or more therapies other than an integrin αᵥβ₃ antagonist, an HMG Co-A reductase inhibitor or a bisphosphonate.

The combination therapies of the invention may be used as a first, second, third, fourth or fifth line of treatment. The invention provides methods for managing, treating or ameliorating one or more symptoms of a disorder associated with abnormal bone resorption in a subject refractory to conventional therapies for such a disorder associated with abnormal bone resorption (e.g., estrogen or progestin), said methods comprising administering to said subject a dose of a prophylactically or therapeutically effective amount of one or more integrin αᵥβ₃ antagonists and a dose of a prophylactically or therapeutically effective amount of one or more HMG Co-A reductase inhibitors and/or a dose of a prophylactically or therapeutically effective amount of one or more bisphosphonates, and optionally, a dose of a prophylactically or therapeutically effective amount of one or more therapies other than an integrin αᵥβ₃ antagonists and an HMG Co-A reductase inhibitor and/or a bisphosphonate.

The invention also provides methods for preventing, managing, treating or ameliorating a disorder associated with abnormal bone resorption or one or more symptoms thereof in a subject refractory to existing single agent therapies for such a disorder associated with abnormal bone resorption, said methods comprising administering to said subject a dose of an effective amount of one or more integrin αᵥβ₃ antagonists, a dose of an effective amount of one or more HMG Co-A reductase inhibitors and optionally a dose of an effective amount of one or more therapies other than an integrin αᵥβ₃ antagonist and an HMG Co-A reductase inhibitor.

The invention also provides methods for managing, treating or ameliorating a disorder associated with abnormal bone resorption or one or more symptoms thereof in a subject refractory to existing single agent therapies, said methods comprising administering to said subject a dose of an effective amount of one or more integrin avf3 antagonists, a dose of an effective amount of one or more bisphosphonates, and optionally, a dose of an effective amount of one or more therapies other than an integrin αᵥβ₃ antagonist and a bisphosphonate. The invention also provides methods for managing or treating a disorder associated with abnormal bone resorption by administering one or more integrin αᵥβ₃ antagonists and one or more HMG Co-A reductase inhibitors and/or one or more bisphosphonates in combination with any other therapy to patients who have proven refractory to other therapies but are no longer on these therapies. The invention also provides alternative methods for the treatment of a disorder associated with abnormal bone resorption where another therapy has proven or may prove too toxic, i.e., results in unacceptable or unbearable side effects, for the subject being treated. For example, one or more integrin αᵥβ₃ antagonists and one or more HMG Co-A reductase inhibitors and/or one or more bisphosphonates may be administered to a subject, wherein the subject is refractory to estrogen therapy. Further, the invention provides methods for preventing the recurrence of a disorder associated with abnormal bone resorption in patients that have been treated and have no disease activity by administering one or more integrin αᵥβ₃ antagonists and one or more HMG Co-A reductase inhibitors and/or one or more bisphosphonates.

Disorders associated with or characterized by abnormal bone resorption include, but are not limited to, Behcet's disease, Gorham-Stout disease; aseptic loosening of a joint; periodontal disease; parathyroid-related disorders (non-limiting examples are primary hyperparathyroidism, lithium therapy and familial hypocalciuric hypercalcemia); malignancy-related disorders (non-limiting examples are solid tumor with metastases, solid tumor with humoral mediation of hypercalcemia, and hematologic malignancies); vitamin D-related disorders (non-limiting examples are vitamin D intoxication, sarcoidosis and other granulomatous diseases, idiopathic hypercalcemia of infancy); and other diseases or disorders associated with high bone turnover (non-limiting examples are hyperthyroidism, immobilization, thiazide, and vitamin A intoxication).

In specific embodiments, the disorders associated with or characterized by abnormal bone resorption that are prevented, managed or treated in accordance with the invention are perodontal diseases (e.g., gingivitis and periodontosis), Gorham Stout disease, osteoporosis (e.g., post-menopausal-glucocorticoid- or inflammation-induced osteoporosis), Paget's disease and aseptic loosening of a joint (e.g., a hip or knee replacement).

In a specific embodiment, the invention provides a method of treating, preventing, managing or ameliorating osteoporosis, or one or more symptoms thereof, comprising administering to a subject an effective amount of an integrin αvβ3 inhibitor (e.g., an antibody that immunospecifically binds to integrin αᵥβ₃; preferably, VITAXIN™ or an antigen-binding fragment thereof; an effective amount of a bisphosphonate and/or an effective amount of an HMG-CoA reductase inhibitor; and an effective amount of one or more agents selected from the group consisting of estradiol, estropipate, transdermal hormone replacement agents and a selective estrogen receptor modulator. In preferred embodiments the estropipate is selected from the group consisting of Cenestin®, Femhrt®, Ogen®, Ortho-Est®g, Premarin®, Premphase®, and Prepro®. In another preferred embodiment, the transdermal hormone replacement therapy is selected from the group consisting of Esclim®, Estraderm®, Vivelle®, and Vivelle-Dot®.

4.6.4 Treatment or Prevention of Disorders Associated with Aberrant Angiogenesis

One or more integrin αᵥβ₃ antagonists or compositions comprising said antagonists in combination with one or more HMG Co-A reductase inhibitors and/or one or more bisphosphonates may be administered to a subject in need thereof to prevent, manage, treat or ameliorate a disorder associated with aberrant angiogenesis or one or more symptoms thereof. One or more integrin αᵥβ₃ antagonists or compositions comprising said antagonists may also be administered in combination with one or more HMG Co-A reductase inhibitors and/or one or more bisphosphonates and one or more other therapies useful for the prevention, management, treatment or amelioration of a disorder associated with aberrant angiogenesis to a subject in need thereof to treat, prevent, manage or ameliorate a disorder associated with aberrant angiogenesis or one or more symptoms thereof. Non-limiting examples of such therapies include analgesics, angiogenesis inhibitors, anti-cancer therapies and anti-inflammatory agents such as those disclosed in Section 4.5 supra, in particular analgesics and angiogenesis inhibitors (e.g., the agents disclosed in Sections 4.5.1, and 4.5.2, supra) to a subject in need thereof to prevent, manage, treat or ameliorate a disorder associated with aberrant angiogenesis or one or more symptoms thereof.

In a specific embodiment, the invention provides a method of preventing, managing, treating or ameliorating a disorder associated with aberrant angiogenesis or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, and optionally, a dose of a prophylactically or therapeutically effective amount of one or more therapies other than integrin αᵥβ₃ antagonists and HMG Co-A reductase inhibitors. In another embodiment, the invention provides a method of preventing, managing, treating or ameliorating a disorder associated with aberrant angiogenesis or one or more symptoms thereof, said method comprising administering to a subject in need thereof a dose of a prophylactically or therapeutically effective amount of one or more integrin αᵥβ₃ antagonists, a dose of prophylactically or therapeutically effective amount of one or more bisphosphonates, and optionally, a dose of a prophylactically or therapeutically effective amount of one or more therapies other than integrin αᵥβ₃ antagonists and bisphosphonates. In another embodiment, the invention provides a method of preventing, managing, treating or ameliorating a disorder associated with aberrant angiogenesis or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, a dose of a prophylactically or therapeutically effective amount of one or more bisphosphonates, and optionally, a dose of a prophylactically or therapeutically effective amount of one or more therapies other than an integrin αᵥβ₃ antagonist, an HMG Co-A reductase inhibitor or a bisphosphonate.

The combination therapies of the invention may be used as a first, second, third, fourth or fifth line of treatment. The invention provides methods for managing, treating or ameliorating one or more symptoms of a disorder associated with aberrant angiogenesis in a subject refractory to conventional therapies for such a disorder associated with aberrant angiogenesis (e.g., angiostatin or vasostatin), said methods comprising administering to said subject a dose of a prophylactically or therapeutically effective amount of one or more integrin αᵥβ₃ antagonists and a dose of a prophylactically or therapeutically effective amount of one or more HMG Co-A reductase inhibitors and/or a dose of a prophylactically or therapeutically effective amount of one or more bisphosphonates, and optionally, a dose of a prophylactically or therapeutically effective amount of one or more therapies other than an integrin αᵥβ₃ antagonists and an HMG Co-A reductase inhibitor and/or a bisphosphonate. The invention also provides methods for preventing, managing, treating or ameliorating a disorder associated with aberrant angiogenesis or one or more symptoms thereof in a subject refractory to existing single agent therapies for such a disorder associated with aberrant angiogenesis, said methods comprising administering to said subject a dose of an effective amount of one or more integrin αᵥβ₃ antagonists, a dose of an effective amount of one or more HMG Co-A reductase inhibitors and optionally, a dose of an effective amount of one or more therapies, other than an integrin αᵥβ₃ antagonist and a bisphosphonate, and optionally a dose of an effective amount of one or more therapies other than an integrin αᵥβ₃ antagonist, and an HMG Co-A reductase inhibitor. The invention also provides methods for managing, treating or ameliorating a disorder associated with aberrant angiogenesis or one or more symptoms thereof in a subject refractory to existing single agent therapies, said methods comprising administering to said subject a dose of an effective amount of one or more integrin αᵥβ₃ antagonists, a dose of an effective amount of one or more bisphosphonates. The invention also provides methods for managing or treating a disorder associated with aberrant angiogenesis by administering one or more integrin αᵥβ₃ antagonists and one or more HMG Co-A reductase inhibitors and/or one or more bisphosphonates in combination with any other therapy to patients who have proven refractory to other therapies but are no longer on these therapies. The invention also provides alternative methods for the treatment of a disorder associated with aberrant angiogenesis where another therapy has proven or may prove too toxic, i.e., results in unacceptable or unbearable side effects, for the subject being treated. For example, one or more integrin αᵥβ₃ antagonists and one or more HMG Co-A reductase inhibitors and/or one or more bisphosphonates may be administered to a subject, wherein the subject is refractory to angiostatin therapy. Further, the invention provides methods for preventing the recurrence of a disorder associated with aberrant angiogenesis in patients that have been treated and have no disease activity by administering one or more integrin αᵥβ₃ antagonists and one or more HMG Co-A reductase inhibitors and/or one or more bisphosphonates.

Diseases or disorders that are associated with or characterized by aberrant angiogenesis include, but not limited to, neoplastic diseases (non-limiting examples are metastases of tumors and leukemia); diseases of ocular neovascularization (non-limiting examples are age-related macular degeneration, diabetic retinopathy, and retinopathy of prematurity, vascular restenosis); skin diseases (non-limiting examples are infantile hemangiomas, verruca vulgaris, psoriasis, basal cell and squamous cell carcinomas, cutaneous melanoma, Kaposi's sarcoma, neurofibromatosis, recessive dystrophic epiderrnolysis bullosa); arthritis (non-limiting examples are rheumatoid arthritis, ankylosing spondylitis, systemic lupus, psoriatic arthropathy, Reiter's syndrome, and Sjogren's syndrome); gynecologic diseases (non-limiting examples are endometriosis, preeclampsia during pregnancy, carcinoma of the ovary, endometrium and cervix); and cardiovascular diseases (non-limiting examples are formation of atherosclerotic plaques, atherosclerosis and coronary artery disease).

4.6.5 Treatment or Prevention of Disorders Associated with Cancer

One or more integrin αᵥβ₃ antagonists or compositions comprising said antagonists in combination with one or more HMG Co-A reductase inhibitors and/or one or more bisphosphonates may be administered to a subject in need thereof to prevent, manage, treat or ameliorate cancer or one or more symptoms thereof. One or more integrin αᵥβ₃ antagonists or compositions comprising said antagonists may also be administered in combination with one or more HMG Co-A reductase inhibitors and/or one or more bisphosphonates and one or more other therapies useful for the prevention, management, treatment or amelioration of cancer to a subject in need thereof to treat, prevent, manage or ameliorate cancer or one or more symptoms thereof. Non-limiting examples of such therapies include analgesics, angiogenesis inhibitors, anti-cancer therapies and anti-inflammatory agents such as those disclosed in Section 4.5 supra, in particular analgesics, TNF-α antagonists, anti-cancer agents, anti-inflammatories, bone metabolism regulating agents, hormones, and metalloproteinases such as the agents disclosed in Sections 4.5.1, 4.5.3, 4.5.6, 4.5.7, 4.5.8, 4.5.10, and 4.5.12, supra, which may be administered to a subject in need thereof to prevent, manage, treat or ameliorate cancer or one or more symptoms thereof.

In a specific embodiment, the invention provides a method of preventing, managing, treating or ameliorating cancer or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, and optionally, a dose of a prophylactically or therapeutically effective amount of one or more therapies other than integrin αᵥβ₃ antagonists and HMG Co-A reductase inhibitors. In another embodiment, the invention provides a method of preventing, managing, treating or ameliorating cancer or one or more symptoms thereof, said method comprising administering to a subject in need thereof a dose of a prophylactically or therapeutically effective amount of one or more integrin αᵥβ₃ antagonists, a dose of prophylactically or therapeutically effective amount of one or more bisphosphonates, and optionally, a dose of a prophylactically or therapeutically effective amount of one or more therapies other than integrin αᵥβ₃ antagonists and bisphosphonates. In another embodiment, the invention provides a method of preventing, managing, treating or ameliorating cancer or one or more symptoms thereof, said method comprising administering to a subject in need thereof a dose of a prophylactically or therapeutically effective amount of one or more integrin αᵥβ₃ antagonists, a dose of a prophylactically or therapeutically effective amount of one or more HMG Co-A reductase inhibitors, a dose of a prophylactically or therapeutically effective amount of one or more bisphosphonates, and optionally, a dose of a prophylactically or therapeutically effective amount of one or more therapies other than an integrin αᵥβ₃ antagonist, an HMG Co-A reductase inhibitor or a bisphosphonate.

The combination therapies of the invention may be used as a first, second, third, fourth or fifth line of treatment. The invention provides methods for managing, treating or ameliorating one or more symptoms of cancer in a subject refractory to conventional therapies for such cancer (e.g., radiation therapy, chemotherapy, or surgery), said methods comprising administering to said subject a dose of a prophylactically or therapeutically effective amount of one or more integrin αᵥβ₃ antagonists and a dose of a prophylactically or therapeutically effective amount of one or more HMG Co-A reductase inhibitors and/or a dose of a prophylactically or therapeutically effective amount of one or more bisphosphonates, and optionally, a dose of a prophylactically or therapeutically effective amount of one or more therapies other than an integrin αᵥβ₃ antagonists and an HMG Co-A reductase inhibitor and/or a bisphosphonate. The invention also provides methods for preventing, managing, treating or ameliorating cancer or one or more symptoms thereof in a subject refractory to existing single agent therapies for such cancer, said methods comprising administering to said subject a dose of an effective amount of one or more integrin αᵥβ₃ antagonists, a dose of an effective amount of one or more HMG Co-A reductase inhibitors and optionally a dose of an effective amount of one or more therapies other than an integrin αᵥβ₃ antagonist and an HMG Co-A reductase inhibitor.

The invention also provides methods for managing, treating or ameliorating cancer or one or more symptoms thereof in a subject refractory to existing single agent therapies, said methods comprising administering to said subject a dose of an effective amount of one or more integrin αᵥβ₃ antagonists, a dose of an effective amount of one or more bisphosphonates, and optionally, a dose of an effective amount of one or more therapies, other than an integrin αᵥβ₃ antagonist and a bisphosphonate. The invention also provides methods for managing or treating cancer by administering one or more integrin αᵥβ₃ antagonists and one or more HMG Co-A reductase inhibitors and/or one or more bisphosphonates in combination with any other therapy to patients who have proven refractory to other therapies but are no longer on these therapies. The invention also provides methods for the management or treatment of a patient having cancer and immunosuppressed by reason of having previously undergone other cancer therapies. A cancer may be determined to be refractory to a therapy when at least some significant portion of the cancer cells are not killed or their cell division arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased. The invention also provides alternative methods for the treatment of cancer or one or more symptoms thereof where another therapy (e.g., chemotherapy, radiation therapy, hormonal therapy, and/or biological therapy/immunotherapy) has proven or may prove too toxic, i.e., results in unacceptable or unbearable side effects, for the subject being treated. For example, one or more integrin αᵥβ₃ antagonists and one or more HMG Co-A reductase inhibitors and/or one or more bisphosphonates may be administered to a subject, wherein the subject is refractory to chemotherapy. Further, the invention provides methods for preventing the recurrence of cancer in patients that have been treated and have no disease activity by administering one or more integrin αᵥβ₃ antagonists and one or more HMG Co-A reductase inhibitors and/or one or more bisphosphonates.

Cancers that can be prevented, treated, or managed in accordance with the methods of the invention include, but are not limited to, neoplasms, tumors (benign or malignant), metastases, or any disease or disorder characterized by uncontrolled cell growth. The cancer may be a primary or metastatic cancer. The cancer may or may not express integrin α_{V}β₃. In a preferred embodiment, the cancer that is being managed, treated or ameliorated in accordance with the methods of the invention is a cancer expressing integrin αᵥβ₃ that has metastasized to the bone. Specific examples of cancers that can be treated, prevented, or managed in accordance with the methods of the invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory mycloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, osteosarcoma, chondrosarcoma, Ewing's sarcoma, Paget's disease of bone, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease), and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America). It is also contemplated that cancers caused by aberrations in apoptosis can also be treated, prevented, managed or ameliorated in accordance with the methods of the invention. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes.

In a preferred embodiment, the cancer that is being prevented, managed, treated or ameliorated in accordance with the method of the invention is prostate cancer, breast cancer (e.g., breast cancer expressing high levels of CD44 and low to undetectable levels of CD24), bone cancer, melanoma, lung cancer and ovarian cancer. In another embodiment, the cancer that is being prevented, managed, treated or ameliorated in accordance with the methods of the invention are metastatic tumors including, but not limited to, tumors that have or may metastasize to the bone (non-limiting examples are prostate, breast and lung cancers that have metastasized or have the potential to metastasize to the bone), tumors that have or may metastasize to the lung, tumors that have or may metastasize to the brain, and tumors that have or may metastasize to other organs or tissues of a subject.

In specific embodiments, patients with breast cancer are administered an effective amount of one or more integrin αᵥβ₃ antagonists, an effective amount of one or more HMG-CoA reductase inhibitors and/or effective amount of one or more bisphosphonates in combination with the administration of an effective amount of one or more other agents useful for breast cancer therapy including but not limited to: doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), Herceptine, tamoxifen, the combination of tamoxifen and cytotoxic chemotherapy. In certain embodiments, patients with metastatic breast cancer are administered an effective amount of one or more HMG CoA reductase inhibitors and/or an effective amount of one or more bisphosphonates in combination with the administration of an effective amount of taxanes such as docetaxel and paclitaxel.

In specific embodiments, patients with prostate cancer are administered an effective amount of one or more integrin αᵥβ₃ antagonists, an effective amount of one or more HMG CoA reductase inhibitors and/or an effective amount of one or more bisphosphonates, in combination with the administration of an effective amount of one or more other agents useful for prostate cancer therapy including but not limited to: external-beam radiation therapy, interstitial implantation of radioisotopes (i.e., I¹²⁵, palladium, Iridium), leuprolide or other LHRH agonists, non-steroidal antiandrogens (flutamide, nilutamide, bicalutamide), steroidal antiandrogens (cyproterone acetate), the combination of leuprolide and flutamide, estrogens such as DES, chlorotrianisene, ethinyl estradiol, conjugated estrogens U.S.P., DES-diphosphate, radioisotopes, such as strontium-89, the combination of external-beam radiation therapy and strontium-89, second-line hormonal therapies such as aminoglutethimide, hydrocortisone, flutamide withdrawal, progesterone, and ketoconazole, low-dose prednisone, or other chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel.

In specific embodiments, patients with ovarian cancer are administered an effective amount of one or more integrin αᵥβ₃ antagonists, an effective amount of one or more HMG CoA reductase inhibitors and/or an effective amount of one or more bisphosphonates, in combination with an effective amount of one or more other agents useful for ovarian cancer therapy including but not limited to: intraperitoneal radiation therapy, such as P³² therapy, total abdominal and pelvic radiation therapy, cisplatin, the combination of paclitaxel (Taxol) or taxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. Included is the treatment of patients with refractory ovarian cancer including administration of: ifosfamide in patients with disease that is platinum-refractory, hexamethylmelamine (HMM) as salvage chemotherapy after failure of cisplatin-based combination regimens, and tamoxifen in patients with detectable levels of cytoplasmic estrogen receptor on their tumors.

In specific embodiments, patients with bone sarcomas are administered an effective amount of one or more integrin αᵥβ₃ antagonists, an effective amount of one or more HMG CoA reductase inhibitors and/or an effective amount of one or more bisphosphonates, in combination with an effective amount of one or more other agents useful for bone sarcoma therapy including but not limited to: doxorubicin, ifosfamide, cisplatin, high-dose methotrexate, cyclophosphamide, etoposide, vincristine, dactinomycin, and surgery.

In specific embodiments, patients with tumor metastatic to bone are administered an effective amount of one or more integrin αᵥβ₃ antagonists, an effective amount of one or more HMG CoA reductase inhibitors and/or an effective amount of one or more bisphosphonates, in combination with an effective amount of one or more other agents useful for bone metastatic tumor therapy including but not limited to: agents or therapies used in treatment of underlying malignancy (non-limiting examples are hormone inhibitors for prostate or breast cancer metastasized to bone and surgery), and radiotherapy (non-limiting examples are strontium 89 and samarium 153, which are bone-seeking radionuclides that can exert anitumor effects and relieve symptoms).

4.7 Compositions and Methods of Administering Compositions

The present invention provides compositions for the treatment, management prevention, and amelioration of inflammatory diseases, autoimmune disorders, disorders associated with abnormal bone metabolism, disorders assocaited with aberaant expression and/or activity of integrin αᵥβ₃ disorders associated with aberrant angiogenesis and cancer, or one or more conditions or symptoms associated therewith. In a specific embodiment, a composition comprises one or more integrin αvβ3 antagonists (e.g., anti-integrin αᵥβ₃ antibodies). In a preferred embodiment, a composition comprises VITAXIN® or an antigen-binding fragment thereof, or an fragment thereof that competes with VITAXIN® or an antibody or an antigen-binding fragment thereof for binding to integrin αᵥβ₃. In another embodiment, a composition comprises one or more bisphosphonates. In another embodiment, a composition comprises one or more HMG-CoA reductase inhibitors. In another embodiment, a composition comprises one or more agents other than an integrin αᵥβ₃ inhibitor, an HMG-CoA reductase inhibitor and/or a bisphosphonate. In another embodiment, the composition comprises one or more integrin αvβ3 antagonists, one or more bisphosphonates, and optionally one or more agents other than an integrin αᵥβ₃ inhibitor, and a bisphosphonate. In another embodiment, the composition comprises one or more integrin αvβ3 antagonists, one or more HMG-CoA reductase inhibitors, and optionally one or more agents other than an integrin αᵥβ₃ inhibitor, and an HMG-CoA reductase inhibitor. In another embodiment, the composition comprises one or more integrin αvβ3 antagonists, one or more bisphosphonates, and one or more HMG-CoA reductase inhibitors, and optionally one or more agents other than an integrin αᵥβ₃ inhibitor, an HMG-CoA reductase inhibitor and/or a bisphosphonate. See Sections 4.5.1 through 4.5.13 supra for examples of other non-limiting therapeutic or prophylactic agents known to be useful in the prevention, treatment or amelioration of inflammatory diseases, autoimmune disorders, disorders associated with abnormal bone metabolism, disorders associated with aberrant angiogenesis and cancers, or one or more conditions or symptoms associated therewith.

In a preferred embodiment, a composition of the invention is a pharmaceutical composition. Such compositions comprise a prophylactically or therapeutically effective amount of one or more prophylactic or therapeutic agents (e.g., an integrin αᵥβ₃ antagonist or other prophylactic or therapeutic agent), and a pharmaceutically acceptable carrier. In other embodiments, such compositions comprise a prophylactically or therapeutically effective amount of an integrin α_{V}β₃ antagonist, a prophylactically or therapeutically effective amount of one or more HMG-CoA reductase inhibitors, and/or a prophylactically or therapeutically effective amount of one or more bisphosphonates, and optionally a prophylactically or therapeutically effective amount of one or more other prophylactic or therapeutic agents that are not integrin αvβ3 antagonists, bisphosphonates, and/or HMG-CoA reductase inhibitors, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions are sterile and in suitable form for administration to a subject, preferably an animal subject, more preferably a mammalian subject, and most preferably a human subject.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering one or more prophylactic or therapeutic agents, care must be taken to use materials to which the prophylactic or therapeutic agents do not absorb.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; International Publication No. WO 99/15154; and International Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention or fragments thereof. See, e.g., U.S. Pat. No. 4,526,938, International publication No. WO 91/05548, International publication No. WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment where the composition of the invention is one or more nucleic acid molecules encoding one or more prophylactic or therapeutic agents, the nucleic acid can be administered in vivo to promote expression of its encoded prophylactic or therapeutic agents, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral (e.g., intravenous, intramuscular, intradermal, intra-tumoral, intra-synovial, and subcutaneous) oral (e.g., inhalation), intranasal, transdermal (topical), transmucosal, intra-tumoral, intra-synovial, and rectal administration. In a specific embodiment, the composition is formulated in accordance with routine procedures as a pharmaceutical composition adapted for intravenous, subcutaneous, intramuscular, oral, intra-tummoral, intra synnovial, intranasal or topical administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions of the invention are to be administered topically, the compositions can be formulated in the form of, e.g., a toothpaste, ointment, cream, transdermal patch, lotion, gel, oral gel, shampoo, spray, aerosol, solution, emulsion, or other form well-known to one of skill in the art. See, e.g., Remington's Pharmaceutical Sciences and Introduction to Pharmaceutical Dosage Forms, 4^{th} ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

If the compositions of the invention are to be administered intranasally, the compositions can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

If the compositions of the invention are to be administered orally, the compositions can be formulated orally in the form of, e.g., gum, tablets, capsules, cachets, gelcaps, solutions, suspensions and the like. Tablets or capsules can be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

The compositions of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions of the invention may also be formulated in rectal compositions such as suppositories or retention enemas, e.g., containing conventional suppository bases such as cocoa butter or other glycerides.

In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the invention provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted, e.g., with water or saline to the appropriate concentration for administration to a subject. Preferably, one or more of the prophylactic or therapeutic agents, or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents, or pharmaceutical compositions of the invention should be stored at between 2 and 8° C in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents, or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container. In preferred embodiments of the invention, VITAXIN® is formulated at 1 mg/ml, 5 mg/ml, 10 mg/ml, and 25 mg/ml for intravenous injections and at 5 mg/ml, 10 mg/ml, 80 mg/ml or 100 mg/ml for repeated subcutaneous administration. In other preferred embodiments of the invention, VITAXIN® is formulated at 1 mg/ml, 5 mg/ml, 10 mg/ml, 20 mg/ml, 40 mg/ml, 60 mg/ml, 80mg/ml, or 100 mg/ml for oral administration. In yet other preferred embodiments of the invention, VITAXIN® is formulated at 5 mg/ml, 10 mg/ml, 15 mg/ml, 20 mg/ml, 50 mg/ml, 100 mg/ml, 200 mg/ml, 300 mg/ml, 400mg/ml, or 500 mg/ml for topical administration.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. In certain preferred embodiments, the pack or dispenser contains one or more unit dosage forms containing no more than 20 mg Periostat™ and 5 mg/ml VITAXIN®.

Generally, the ingredients of the compositions of the invention are derived from a subject that is the same species origin or species reactivity as recipient of such compositions. Thus, in a preferred embodiment, human or humanized antibodies are administered to a human patient for therapy or prophylaxis.

4.7.1 Gene Therapy

In a specific embodiment, nucleic acids comprising sequences encoding one or more prophylactic or therapeutic agents, are administered to treat, manage, prevent or ameliorate inflammatory diseases, autoimmune disorders, disorders associated with aberrant expression and/or activity of integrin αᵥβ₃, disorders associated with abnormal bone metabolism, disorders associated with aberrant angiogenesis and cancers or one or more symptoms thereof by way of gene therapy. Gene therapy refers to therapy performed by the administration to a subject of an expressed or expressible nucleic acid. In this embodiment of the invention, the nucleic acids produce their encoded prophylactic or therapeutic agent that mediates a prophylactic or therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, N.Y. (1993); and Kriegler, Gene Transfer and Expression, A Laboratory Manual, Stockton Press, N.Y. (1990).

In a preferred aspect, a composition of the invention comprises nucleic acids encoding a prophylactic or therapeutic agent, said nucleic acids being part of an expression vector that expresses the prophylactic or therapeutic agent in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the prophylactic or therapeutic agent coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In certain embodiments, the prophylactic or therapeutic agent expressed is an integrin α_{V}β₃ antagonist. In other embodiments the prophylactic or therapeutic agent expressed is an agent known to be useful for, or has been or is currently being used in the prevention, management, treatment or amelioration of inflammatory diseases, autoimmune disorders, disorders associated with aberrant expression and/or activity of integrin αᵥβ₃, disorders associated with abnormal bone metabolism, disorders associated with aberrant angiogenesis and cancers, or one or more symptoms thereof. In a preferred embodiment, the prophylactic or therapeutic agent expressed is VITAXIN®.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or by a matrix with in situ scaffolding in which the nucleic acid sequence is contained (see, e.g., European Patent No. EP 0 741 785 B1 and U.S. Pat. No. 5,962,427), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Publication Nos. WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding a prophylactic or therapeutic agent are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication W094/12649; and Wang et al., 1995, Gene Therapy 2:775-783. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include, but are not limited to, epithelial cells; endothelial cells; keratinocytes; fibroblasts; muscle cells; osteoclasts; hepatocytes; blood cells such as T lymphocytes, B lymphocytes, natural killer (NK) cells, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the subject.

In an embodiment in which recombinant cells are used in gene therapy, nucleic acid sequences encoding a prophylactic or therapeutic agent are introduced into the cells such that they are expressible by the cells or their progeny, and the recombinant cells are then administered in vivo for prophylactic or therapeutic effect. In a specific embodiment, stem or progenitor cells are used. Any stem and/or progenitor cells which can be isolated and maintained in vitro can potentially be used in accordance with this embodiment of the present invention (see e.g., International Publication No. WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises a constitutive, tissue-specific, or inducible promoter operably linked to the coding region. In a preferred embodiment, the nucleic acid to be introduced for purposes of gene therapy comprises an inducible promoter operably linked to the coding region, such that expression of the nucleic acid is controllable by controlling the presence or absence of the appropriate inducer of transcription.

4.7.2 Dosages & Frequency of Administration

The amount of the composition of the invention which will be effective in the treatment, management, prevention or amelioration of inflammatory diseases, autoimmune disorders, disorders associated with aberrant expression and/or activity of integrin αᵥβ₃, disorders associated with abnormal bone metabolism, disorders associated with aberrant angiogenesis and cancers, or one or more conditions or symptoms associated therewith, can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from those well-known in the art, including for single agent therapies set forth in Sections 4.5.1 through 4.5.13 supra. Additionally, effective doses may be extrapolated from dose-response curves derived from in vitro or animal model test systems. Some non-limiting examples of bone resorption cell cultures that may be used to determine effective doses include osteoclasogenesis cell cultures, purified osteoclasts, partially-purified osteoclasts, unpurified osteoclasts, purified pre-osteoclasts, partially-purified pre-osteoclasts, unpurified pre-osteoclasts, purified osteoclast-like cells, partially-purified osteoclast-like cells, unpurified osteoclast-like cells, and mixtures thereof. In a preferred embodiment, lower doses of each ingredient of a combination therapy is used in the combination therapy.

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In a preferred embodiment, the dose of an antibody or antibody fragment that immunospecifically binds to integrin α_{V}β₃ (e.g., VITAXIN® or a fragment thereof) is 0.1 to 10 mg/kg/week, preferably 1 to 9 mg/kg/week, more preferably 2 to 8 mg/week, even more preferably 3 to 7 mg/kg/week, and most preferably 4 to 6 mg/kg/week. In another embodiment, a subject, preferably a human, is administered one or more doses of a prophylactically or therapeutically effective amount of an antibody or antibody fragment that immunospecifically binds to integrin α_{V}β₃ (e.g., VITAXIN® or a fragment thereof), wherein the dose of a prophylactically or therapeutically effective amount of the antibody or antibody fragment administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses. In another embodiment, a subject, preferably a human, is administered one or more doses of a prophylactically or therapeutically effective amount of an antibody or antibody fragment that immunospecifically binds to integrin α_{V}β₃ (e.g., VITAXIN® or a fragment thereof), wherein the dose of a prophylactically or therapeutically effective amount of the antibody or antibody fragment administered to said subject is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

In specific embodiments, an antibody or antibody fragment that immunospecifically binds to integrin α_{V}β₃ (e.g., VITAXIN® or a fragment thereof) is administered in a dosing regimen that maintains the plasma concentration of the antibody immunospecific for integrin α_{V}β₃ at a desirable level (e.g., about 0.1 to about 100 μg/ml), which continuously blocks the integrin αᵥβ₃ activity. In a specific embodiment, the plasma concentration of the antibody is maintained at 0.2 μg/ml, 0.5 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 15 μg/ml, 20 μg/ml, 25 μg/ml, 30 μg/ml, 35 μg/ml, 40 μg/ml, 45 μg/ml or 50 μg/ml. The plasma concentration that is desirable in a subject will vary depending on several factors, including but not limited to, the nature of the disease or disorder, the severity of the disease or disorder and the condition of the subject. Such dosing regimens are especially beneficial in prevention, treatment, management and amelioration of a chronic disease or disorder.

In another embodiment, an antagonist of integrin α_{V}β₃ is administered to a subject with a disease or disorder that associated with bone resportion using a dosing regimen that maintains the plasma concentration of the antagonist at a level that blocks at least 40%, preferably at least 50%, at least 55%, at least 60%, at least 65%, at least 70%, at least 75%, at least 80%, at least 85%, at least 90% or at least 95% of bone resorption.

In specific embodiments, an antibody or antibody fragment that immunospecifically binds to integrin α_{V}β₃ (e.g., in particular, a conjugated antibody or antibody fragment immunospecific for integrin α_{V}β₃) is administered intermittently (e.g., one every other week, once every two weeks, once every three weeks, or once a month). As used herein, “a conjugated antibody or antibody fragment” refers to an antibody or antibody fragment that is conjugated to another moiety, including but not limited to, a heterologous peptide, polypeptide, another antibody or antibody fragment, a marker sequence, a diagnostic agent, a therapeutic agent, a radioactive metal ion, a polymer, albumin, and a solid support.

For other prophylactic or therapeutic agents administered to a patient, the typical doses of various such agents are well-known in the art and examples are provided in Tables 4 and 5. The invention provides for any method of administrating lower doses of known prophylactic or therapeutic agents than previously thought to be effective for the prevention, treatment, management or amelioration of cancer or one or more symptoms thereof. Preferably, lower doses of other therapeutic agents are administered in combination with lower doses of integrin α_{V}β₃ antagonists.

The dosages of prophylactic or therapeutically agents are described in the Physicians' Desk Reference (57^{th} ed., 2003).

4.8 Characterization and Demonstration of Therapeutic or Prophylactic Utility

Several aspects of the compositions and combination therapies of the invention are preferably tested in vitro, in a cell culture system, and in an animal model organism, such as a rodent animal model system, for the desired therapeutic activity prior to use in humans. For example, assays which can be used to determine whether administration of a specific composition or combination therapy of the invention is indicated, include cell culture assays in which a patient tissue sample is grown in culture, and exposed to or otherwise contacted with a composition of the invention, and the effect of such composition upon the tissue sample is observed. The tissue sample can be obtained by biopsy from the patient. This test allows the identification of the therapeutically most effective prophylactic or therapeutic agent(s) for each individual patient. In various specific embodiments, in vitro assays can be carried out with representative cells of cell types involved in an autoimmune disorder, an inflammatory disorder, a disorder associated with aberrant expression and/or activity of integrin αᵥβ₃, a disorder associated with abnormal bone metabolism, a disorder associated with aberrant angiogenesis, or cancer (e.g., endothelial cells, cancer cells, activated T cells, osteoclasts and B cells), to determine if a composition of the invention has a desired effect upon such cell types. A lower level of proliferation or survival of the contacted cells indicates that the composition of the invention is effective to treat, manage, prevent or ameliorate the condition in the patient. Alternatively, instead of culturing cells from a patient, a composition of the invention may be screened using cells of a tumor or malignant cell line or an endothelial cell line. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos and myc) or cell cycle markers; cell viability can be assessed by trypan blue staining, differentiation can be assessed visually based on changes in morphology, etc.

The binding specificity, affinity and functional activity of the antibody or antibody fragment in the compositions of the invention can be characterized in various in vitro binding and cell adhesion assays known in the art, including but limited to, those that are disclosed in International Publication Nos. WO 00/78815 and WO 02/070007, U.S. Pat. No. 6,248,326, U.S. Pat. No. 6,472,403, Pecheur et al., The FASEB J. 16(10):1266-8 (2002), Almed et al., The Journal of Histochemistry & Cytochemistry 50:1371-1379 (2002), all of which are incorporated herein by reference.

The binding specificity of the antibody or antibody fragment in the compositions of the invention can be assessed by measuring binding to integrin αᵥβ₃ and its crossreactivity to other αᵥ- or β₃-containing integrins. Specifically, binding specificity can be assessed by measuring binding to α_{11b}β₃, the major integrin expressed on platelets, and to αᵥβ₅, an integrin found prevalent on endothelial cells and connective tissue cell types. Briefly, to determine crossreactivity, integrins are coated onto an ELISA plate and a series of antibody dilutions are measured for antibody binding activity against integrin αᵥβ₃ and the other integrins. The integrins αᵥβ₃ and α_{vβ}₅ can be isolated by know techniques in the art, e.g., by affinity chromatography as described in Cheresh, 1987, Proc. Natl. Acad. Sci. USA 84:6471-6475, and Cheresh and Spiro, 1987, J. Biol. Chem. 262:17703-17711. In a specific embodiment, an anti-αᵥβ₃ antibody affinity column is used to isolate αᵥβ₃ from an octylglucoside human placental lysate, whereas an anti-αᵥ affinity column is used to isolate αᵥβ₅ from the integrin αᵥβ₃ depleted column flow through. Antibody binding activity is assessed by ELISA using a goat anti-human IgG-alkaline phosphatase conjugate. A purified human IgG₁ antibody can be used as a control.

In another embodiment, the binding affinity and specificity are assessed in a competitive binding assay with the parental anti-integrin αᵥβ₃ antibody against integrin αᵥβ₃. Competitive binding is measured in an ELISA assay. Binding of the antibody is determined in the presence of increasing concentrations of antibody competitor. Alternatively, the control competitor antibody is again a human IgG₁.

In another embodiment, binding affinity and specificity are assessed by measuring the inhibitory activity of the antibody on integrin αᵥβ₃ binding to fibrinogen. Briefly, integrin αᵥβ₃ is plated onto ELISA plates. Inhibitory activity of the antibody is determined by measuring the amount of bound biotinylated fibrinogen in the presence of increasing concentrations of antibody or control antibody. Streptavidin alkaline phosphatase is used to detect the bound fibrinogen.

In another embodiment, the specificity of the antibody binding is assessed by the inhibition of integrin αᵥβ₃ binding in cell adhesion assays. Endothelial cell adhesion events are an important component in the angiogenic process and inhibition of αᵥβ₃ is known to reduce the neovascularization of tumors and thereby reduce the rate of tumor growth. The inhibition of αᵥβ₃-mediated cell attachment by anti-integrin αᵥβ₃ antibody in these assays is indicative of the inhibitory activity expected when this antibody is used in situ or in vivo. Briefly, integrin αᵥβ₃-positive M21 melanoma cells grown in RPMI containing 10% FBS are used for these cell binding assays. Cells are released from the culture dish by trypsinization and re-suspended in adhesion buffer at a concentraton of 4×10⁵ cells/ml. The antibody and the control antibody are diluted to the desired concentration in 250 μl adhesion buffer (10 mM Hepes, 2 mM MgCl₂, 2 mM CaCl₂, 0.2 mM MnCl₂, and 1% BSA in Hepes buffered saline at pH 7.4) and added to wells of a 48-well plate precoated with fibrinogen. Each well is coated with 200 μl fibrinogen at a concentration of 10 μg/ml for 1 hour at 37° C. For the assay, an equal volume of cells (250 μl) containing the antibody or isotype matched control antibody is added to each of the wells, mixed by gentle shaking and incubated for 20 minutes at 37° C. Unbound cells are removed by washing with adhesion buffer until no cells remained in control wells coated with BSA alone. Bound cells are visulized by staining with crystal violet which is subsequently extracted with 100 μl acetic acid (10%) and quantitated by determining the absorbance of the solubilized dye at 560 nm.

In another embodiment, the inhibitory activity of an antibody that immunospecifically binds to integrin αᵥβ₃ is also tested in an endothelial cell migration assay. In this regard, the Transwell cell migration assay is used to assess the ability of Vitaxin to inhibit endothelial cell migration (Choi et al., 1994, J. Vascular Surg., 19:125-134 and Leavesly et al., 1993, J. Cell Biol., 121:163-170). Briefly, human umbilical vein endothelial cells in log phase and at low passage number are harvested by gentle trypsinization, wash and resuspend at a concentration of 2×10⁶ cells/ml in 37° C. HBS containing 1% BSA (20 mM Hepes, 150 mM NaCl, 1.8 mM MgCl₂, 1.8 mM CaCl₂, 5 mM KCl, and 5 mM glucose, pH 7.4). Antibodies are diluted to 10 μl/ml from stock solutions. Antibodies are added to cells in a 1:1 dilution (final concentration of antibodies=5 μg/ml; final concentration of cells=1×10⁶ cells/ml) and incubated on ice for 10-30 minutes. The cell/antibody suspensions (200 μl to each compartment) are then added to the upper compartments of a Transwell cell culture chamber, the lower compartments of which had been coated with 0.5 ml of 10 μg/ml vitronectin (in HBS). Vitronectin serves as the chemoattractant for the endothelial cells. The chambers are placed at 37° C. for 4 hours to allow cell migrate to occur. Visualization of cell migration is performed by first removing the remaining cells in the upper compartment with a cotton swab. Cells that had migrated to the lower side of insert are stained with crystal violet for 30 minutes, followed by solubilization in acetic acid and the absorbance of the dye is measure at a wavelength of 550 nm. The amount of absorbance is directly proportional to the number of cells that have migrated from the upper to the lower chamber.

In a preferred embodiment, BIAcore kinetic analysis is used to determine the binding on and off rates of antibodies or fragments thereof to integrin αᵥβ₃. BIAcore kinetic analysis comprises analyzing the binding and dissociation of integrin αᵥβ₃ from chips with immobilized antibodies or fragments thereof on their surface.

Additional examples of in vitro assays, e.g., Western blotting analysis, flow cytometric analysis, cell adhesion assay to cortical bone and extracellular matrix proteins, cell migration assay, cell invasion assay, and cell proliferation assay, can be found in Pecheur et al., The FASEB J. 16(10):1266-8 (2002), of which the entire text is incorporated herein by reference.

The compositions and combinations therapy of the invention can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, the compositions and combination therapies of the invention are tested in a mouse model system. Such model systems are widely used and well-known to the skilled artisan such as the SCID mouse model or transgenic mice where a mouse integrin α_{V}β₃ is replaced with the human integrin α_{V}β₃, nude mice with human xenografts, animal models wherein an antibody or fragment thereof that immunospecifically binds to integrin α_{V}β₃ recognizes the same target as VITAXIN,® such as hamsters, rabbits, etc. known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties. The compositions and combination therapies of the invention can be administered repeatedly. Several aspects of the procedure may vary.

Various animal models known in the art that are relevant to a targeted disease or disorder, e.g., inflammatory diseases, autoimmune diseases, diseases or disorders associated with aberrant bone metabolism and/or aberrant angiogenesis, disorders associated with aberrant integrin αᵥβ₃, expression and/or activity, or cancer can be used, including but not limited to, those that are disclosed in International Publication Nos. WO 00/78815, U.S. Pat. No. 6,248,326, U.S. Pat. No. 6,472,403, Pecheur et al., The FASEB J. 16(10):1266-8 (2002), Almed et al., The Journal of Histochemistry & Cytochemistry 50:1371-1379 (2002), all of which are incorporated herein by reference.

In one embodiment, inhibition of tumor growth by a composition and a combination therapy of the invention is tested in two animal models. The first model measures angiogenesis in the chick chorioallantoic membrane (CAM). This assay is a well recognized model for in vivo angiogenesis because the neovascularization of whole tissue is occurring. Specifically, the assay measures growth factor induced angiogenesis of chicken CAM vessels growing toward the growth factor-impregnated filter disk or into the tissue grown on the CAM. Inhibition of neovascularization is based on the amount and extent of new vessel growth or on the growth inhibition of tissue on the CAM. The assay has been described in detail by others and has been used to measure neovascularization as well as the neovascularization of tumor tissue (Ausprunk et al., 1980, Am. J. Pathol., 79:597-618 (1975); Ossonski et al., Cancer Res., 40:2300-2309; Brooks et al., 1994, Science, 264:569-571 and Brooks et al., 1994, Cell, 79:1157-1164. Briefly, for growth factor induced angiogenesis filter disks are punched from #1 Whatman Qualitative Circles using a skin biopsy punch. Disks are first sterilized by exposure to UV light and then saturated with varying concentrations of TNF-α of HBSS as a negative control (for at least 1 hour) under sterile conditions. Angiogenesis is induced by placing the saturated filter disks on the CAMs. Inhibition of angiogenesis is performed by treating the embryos with various amounts of Vitaxin and controls (antibody or purified human IgG₁). The treatments are performed by intravenous injection approximately 24 hours after disk placement. After 48 hours, CAMs are dissected and angiogenesis is scored on a scale of 1-4. HBSS saturated filter disks are used as the negative control, representing angiogenesis that may occur in response to tissue injury in preparing CAMs, and, values for these CAMs are subtracted out as background. Purified human IgG₁ is used as the negative control for injections since VITAXIN® is of the human IgG₁ subclass.

In addition to the above described CAM assay using growth factor-induced neovascularization, additional assays can be performed utilizing tumor-induced neovascularization. For these assays, angiogenesis is induced by transplanting of αᵥβ₃-negative tumor fragments into the CAMs. The use of αᵥβ₃-negative tumor fragments ensures that any inhibition of tumor growth is due to the inhibition of αᵥβ₃-mediated neovascularization by CAM-derived endothelial cells and not to adhesion events mediated by αᵥβ₃ present on the tumor cells. Inhibition of tumor growth is assessed by placing a single cell suspension of FG (8×10⁶ cells, pancreatic carcinoma) and Hep-3 cells (5×10⁵ cells, laryngeal carcinoma) onto CAMs in 30 μl. One week later, tumors are removed and cut into approximately 50 mg fragments at which time they are placed onto new CAMs. After 24 hours of this second placement, embryos are injected intravenously with Vitaxin or human IgG₁ as a negative control. The tumors are allowed to grow for about 7 days following which they are removed and weighed.

In a second animal model, the inhibition of Vx2 carcinoma cells in rabbits is used as a measure of inhibitory effect on tumors of a composition and a combination therapy of the invention. The Vx2 carcinoma is a transplantable carcinoma derived from a Shope virus-induced papilloma. It was first described in 1940 and has since been used extensively in studies on tumor invasion, tumor-host interactions and angiogenesis. The Vx2 carcinoma is fibrotic in nature, highly aggressive, and exhibits features of an anaplastic type carcinoma. Propagation of Vx2 tumor is accomplished through serial transplantation in donor rabbits. Following subcutaneous transplantation, it has been reported that after an initial inflammatory reaction, host repair mechanisms set in between days 2 and 4. This repair mechanism is characterized by the formation of new connective tissue and the production of new capillaries. The newly formed capillaries are restricted to the repair zone at day 4, however, by day 8 they have extended to the outer region of the tumor. These characteristics and the pharmacokinetics of a composition and a combination therapy of the invention in rabbits can be used to determine initial doses and scheduling of treatments for these experiments.

Growth of Vx2 tumors in the above animal model is used to study the effect of a composition and a combination therapy of the invention after early administration on primary tumor growth in rabbits implanted subcutaneously with Vx2 carcinoma. Briefly, Vx2 tumors (50 mg) are transplanted into the inner thigh of rabbits through an incision between the skin and muscle. Measurements of the primary tumor are taken throughout the experiment through day 25.

In another embodiment, BALB/c nu/nu mice are used as animal models to study different diseases, especially those associated with aberrant bone metabolism and/or aberrant angiogenesis. Different cell lines (e.g., CHO, or a type of cancer cells such as breast cancer cells) expressing αᵥβ₃ in various forms can be injected intravenously into the nude mice. See Pecheur et al., supra. For example, CHO cells are transfected with various cDNA constructs of αᵥβ₃ (e.g., wild-type, mutated forms) and injected intravenously into nude mice. The effects of αᵥβ₃ (with various level of activity because of the mutations) and anti-αᵥβ₃ antibodies on bone metastases can be assessed by, e.g., radiograph, histological examination of bone tissue or statistical analysis.

In another embodiment, animals (healthy or previously constructed animal models) in space environment (e.g., space shuttle) can be used to assess the antibodies of the invention. Since astronauts in long space flights have been shown to lose bone density in a way that is similar to osteoporosis patient, but ten times faster than in people who have the advantage of Earth's gravity (see BioWorld Today, 14:13, Jan. 21, 2003), animals in space environment are ideal osteoporosis model for determining the effects of antibodies or antibody fragments of the invention on osteoporosis or other diseases related to aberrant bone metabolism and/or aberrant angiogenesis.

In another embodiment, SCID mice with subcutaneously implanted human bone fragments (SCID-human-bone model) are used as an animal model to assess the effects of the antibodies or antibody fragments of the invention on diseases associated with aberrant bone metabolism and/or aberrant angiogenesis. For examples, cancer cells (e.g., human prostate cancer cells) are injected directly into human bone fragments in the animal model. At the same time, antibody treatment is initiated. The effects of the antibody or antibody fragment of the invention on bone metastases or angiogenesis can be assessed by comparing to a control group. See Nemeth et al., 2002, Clinical & Experimental Metastasis, 19 (Supp. 1):47.

The efficacy of the pharmaceutical compositions and combination therapies of the invention can be assessed using assays that determine bone loss. Animal models such as ovariectomy-induced bone resorption mice, rat and rabbit models are known in the art for obtaining dynamic parameters for bone formation. Using methods such as those described by Yositake et al. or Yamamoto et al., bone volume is measured in vivo by microcomputed tomography analysis and bone histomorphometry analysis. Yoshitake et al., 1999, “Osteopontin-Deficient Mice Are Resistant to Ovariectomy-Induced Bone Resorption,” Proc. Natl. Acad. Sci. 96:8156-8160; Yamamoto et al., 1998, “The Integrin Ligand Echistatin Prevents Bone Loss in Ovariectomized Mice and Rats,” Endocrinology 139(3):1411-1419, both incorporated herein by reference in their entirety.

The anti-inflammatory activity of the compositions and combination therapies of the invention can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al.(eds.), Chapter 30 (Lee and Febiger, 1993). Experimental and spontaneous animal models of inflammatory arthritis and autoimmune rheumatic diseases can also be used to assess the anti-inflammatory activity of the composition and combination therapies of the invention.

The principle animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al.(eds.), Chapter 30 (Lee and Febiger, 1993), incorporated herein by reference in its entirety.

The anti-inflammatory activity of the compositions and combination therapies of the invention can be assessed using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al, “Carrageenan-Induced Arthritis in the Rat,” Inflammation, 24(2): 141-155, (2000). Also commonly used are zymosan-induced inflammation animal models as known and described in the art.

The anti-inflammatory activity of the compositions and combination therapies of the invention can also be assessed by measuring the inhibition of carrageenan-induced paw edema in the rat, using a modification of the method described in Winter C. A. et al., 1962, “Carrageenan-Induced Edema in Hind Paw of the Rat as an Assay for Anti-inflammatory Drugs” Proc. Soc. Exp. Biol Med. 111, 544-547. This assay has been used as a primary in vivo screen for the anti-inflammatory activity of most NSAIDs, and is considered predictive of human efficacy. The anti-inflammatory activity of the composition and combination therapies of the invention is expressed as the percent inhibition of the increase in hind paw weight of the test group relative to the vehicle dosed control group.

In a specific embodiment of the invention where the experimental animal model used is adjuvant-induced arthritis rat model, body weight can be measured relative to a control group to determine the anti-inflammatory activity of the compositions and and combination therapies of the invention. Alternatively, the efficacy of the compositions and combination therapies of the invention can be assessed using assays that determine bone loss. Animal models such as ovariectomy-induced bone resorption mice, rat and rabbit models are known in the art for obtaining dynamic parameters for bone formation. Using methods such as those described by Yositake et al. or Yamamoto et al., bone volume is measured in vivo by microcomputed tomography analysis and bone histomorphometry analysis. Yoshitake et al., 1999, “Osteopontin-Deficient Mice Are Resistant to Ovariectomy-Induced Bone Resorption,” Proc. Natl. Acad. Sci. 96:8156-8160; Yamamoto et al., 1998, “The Integrin Ligand Echistatin Prevents Bone Loss in Ovariectomized Mice and Rats,” Endocrinology 139(3):1411-1419, both incorporated herein by reference in their entirety.

Additionally, animal models for inflammatory bowel disease can also be used to assess the efficacy of the compositions and combination therapies of the invention (Kim et al., 1992, Scand. J. Gastroentrol. 27:529-537; Strober, 1985, Dig. Dis. Sci. 30(12 Suppl):3S-IOS). Ulcerative cholitis and Crohn's disease are human inflammatory bowel diseases that can be induced in animals. Sulfated polysaccharides including, but not limited to amylopectin, carrageen, amylopectin sulfate, and dextran sulfate or chemical irritants including but not limited to trinitrobenzenesulphonic acid (TNBS) and acetic acid can be administered to animals orally to induce inflammatory bowel diseases.

Animal models for autoimmune disorders can also be used to assess the efficacy of the compositions and combination therapies of the invention. Animal models for autoimmune disorders such as type 1 diabetes, thyroid autoimmunity, sytemic lupus eruthematosus, and glomerulonephritis have been developed (Flanders et al., 1999, Autoimmunity 29:235-246; Krogh et al., 1999, Biochimie 81:511-515; Foster, 1999, Semin. Nephrol. 19:12-24).

Further, any assays known to those skilled in the art can be used to evaluate the prophylactic and/or therapeutic utility of the compositions and combination therapies of the inventions disclosed herein for autoimmune disorders, inflammatory diseases, disorders associated with aberrant expression and/or activity of integrin αᵥβ₃, diseases or disorders associated with aberrant bone metabolism or aberrant angiogenesis, and/or cancers.

The effect of the compositions and combination therapies of the invention on peripheral blood lymphocyte counts can be monitored/assessed using standard techniques known to one of skill in the art. Peripheral blood lymphocytes counts in a subject can be determined by, e.g., obtaining a sample of peripheral blood from said subject, separating the lymphocytes from other components of peripheral blood such as plasma using, e.g., Ficoll-Hypaque (Pharmacia) gradient centrifugation, and counting the lymphocytes using trypan blue. Peripheral blood T-cell counts in subject can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen such as CD3, CD4, and CD8 which is conjugated to FITC or phycoerythrin, and measuring the number of T-cells by FACS.

The toxicity and/or efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The compositions and combination therapies of the invention that exhibit large therapeutic indices are preferred. While compositions and combination therapies of the invention that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

4.9 Kits

The invention provides a pharmaceutical pack or kit comprising one or more containers filled with an integrin α_{V}β₃ antagonist, and a bisphosphonate, HMG-CoA reductase inhibitor and/or one or more therapeutic or prophylactic agents other than an integrin α_{V}β₃ antagonist, bisphosphonate, and/or HMG-CoA reductase inhibitor. The pharmaceutical pack or kit may further comprises one or more other prophylactic or therapeutic agents useful for the treatment of a disease or disorder. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises an integrin α_{V}β₃ antagonist and a bisphosphonate, HMG-CoA reductase inhibitor and/or one or more therapeutic or prophylactic agents other than an integrin α_{V}β₃ antagonist, bisphosphonate, HMG-CoA reductase inhibitor in one or more containers. The kit may further comprises one or more other prophylactic or therapeutic agents useful for the treatment of cancer, an inflammatory disease, an autoimmune disorder, a disorder associated with aberrant expression and/or activity of integrin αᵥβ₃ a disorder associated with abnormal bone metabolism, and/or a disorder associated with aberrant angiogenesis, in one or more containers. Preferably, the integrin α_{V}β₃ antagonist is VITAXIN®. Examples of such agents are disclosed above in Section 4.5.

The invention also provides a pharmaceutical pack or kit comprising one or more containers filled with an integrin α_{V}β₃ antagonist conjugated to another moiety, including but not limited to, a heterologous polypeptide, peptide or protein, a large molecule, a small molecule, a marker sequence, a diagnostic or detectable agent, a therapeutic agent, a radioactive metal ion, a second antibody, and a solid support. The pharmaceutical pack or kit may further comprises one or more other prophylactic or therapeutic agents useful for the treatment of a cancer. In preferred embodiments, the integrin α_{V}β₃ antagonist is VITAXIN®. In another preferred embodiment, the integrin α_{V}β₃ antagonist conjugated to another moiety is VITAXIN® conjugated to HMG-CoA reductase and/or a bisphosphonate.

4.10 Articles of Manufacture

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, intratumoral, intra-synovial, topical or mucosal delivery.

In a specific embodiment, the unit dosage form is suitable for intravenous, intramuscular, intratumoral, intra-synovial, or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures (such as methods for monitoring mean absolute lymphocyte counts, tumor cell counts, calcium concentration, and tumor size) and other monitoring information.

More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material.

In a specific embodiment, an article of manufacture comprises packaging material and a pharmaceutical agent and instructions contained within said packaging material, wherein said pharmaceutical agent is an integrin αvβ₃ antagonist and a pharmaceutically acceptable carrier, and said instructions indicate a dosing regimen for preventing, treating or managing a subject with cancer, an inflammatory disease, an autoimmune disorder, a disorder associated with aberrant expression and/or activity of integrin αᵥβ₃, a disorder associated with abnormal bone metabolism, and/or a disorder associated with aberrant angiogenesis. In another embodiment, an article of manufacture comprises packaging material and a pharmaceutical agent and instructions contained within said packaging material, wherein said pharmaceutical agent comprises an integrin αvβ₃ antagonist, a prophylactic or therapeutic agent other than an integrin αvβ₃ antagonist and a pharmaceutically acceptable carrier, and said instructions indicate a dosing regimen for preventing, treating or managing a subject with a cancer, an inflammatory disease, an autoimmune disorder, a disorder associated with aberrant expression and/or activity of integrin αᵥβ₃, a disorder associated with abnormal bone metabolism, and/or a disorder associated with aberrant angiogenesis. In another embodiment, an article of manufacture comprises packaging material and two or more pharmaceutical agents and instructions contained within said packaging material, wherein said first pharmaceutical agent comprises an integrin αvβ₃ antagonist and a pharmaceutically acceptable carrier and said second pharmaceutical agent comprises a bisphosphonate or HMG-CoA reductase inhibitor and a pharmaceutically acceptable carrier, and optionally a third pharmaceutical agent comprises one or more prophylactic or therapeutic agents other than an integrin αvβ₃ antagonist, and said instructions indicate a dosing regimen for preventing, treating or managing a subject with a cancer, an inflammatory disease, an autoimmune disorder, a disorder associated with aberrant expression and/or activity of integrin αᵥβ₃, a disorder associated with abnormal bone metabolism, and/or a disorder associated with aberrant angiogenesis.

The present invention provides that the adverse effects that may be reduced or avoided by the methods of the invention are indicated in informational material enclosed in an article of manufacture for use in preventing, treating or ameliorating one or more symptoms associated with cancer. Adverse effects that may be reduced or avoided by the methods of the invention include but are not limited to vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation. Since some of the therapies may be immunosuppressive, prolonged immunosuppression may increase the risk of infection, including opportunistic infections. Prolonged and sustained immunosuppression may also result in an increased risk of developing certain types of cancer.

Further, the information material enclosed in an article of manufacture for use in preventing, treating or ameliorating an inflammatory disease, an autoimmune disorder, a disorder associated with aberrant expression and/or activity of integrin αᵥβ₃, a disorder associated with abnormal bone metabolism, a disorder associated with aberrant angiogenesis or cancer, or one or more symptoms associated therewith can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

The information material can also indicate that cytokine release syndrome is an acute clinical syndrome, temporally associated with the administration of certain antibodies. Cytokine release syndrome has been attributed to the release of cytokines by activated lymphocytes or monocytes. The clinical manifestations for cytokine release syndrome have ranged from a more frequently reported mild, self-limited, “flu-like” illness to a less frequently reported severe, life-threatening, shock-like reaction, which may include serious cardiovascular, pulmonary and central nervous system manifestations. The syndrome typically begins approximately 30 to 60 minutes after administration (but may occur later) and may persist for several hours. The frequency and severity of this symptom complex is usually greatest with the first dose. With each successive dose, both the incidence and severity of the syndrome tend to diminish. Increasing the amount of a dose or resuming treatment after a hiatus may result in a reappearance of the syndrome. As mentioned above, the invention encompasses methods of treatment and prevention that avoid or reduce one or more of the adverse effects discussed herein.

4.11 Methods of Producing Antibodies

The antibodies that immunospecifically bind to an antigen (e.g., integrin αᵥβ₃) can be produced by any method known in the art for the synthesis of antibodies, in particular, by chemical synthesis or preferably, by recombinant expression techniques.

Polyclonal antibodies immunospecific for an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC™. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention encompasses methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/O1 134; International publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Completely human antibodies and humanized antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,8 16397, and 6,311,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immuoglobulin. A humanized antibody comprises substantially all of at least one, and typically two, variable domains (Fab, Fab′, F(ab′)₂, Fabc, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fe), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and most preferably greater than 95%. Humanized antibody can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Patent No. 5,565,332), and techniques disclosed in, e.g., U.S. Pat. No. 6,407,213, U.S. Pat. No. 5,766,886, WO 9317105, Tan et al., J. Immunol. 169:1119-25 (2002), Caldas et al., Protein Eng. 13(5):353-60 (2000), Morea et al., Methods 20(3):267-79 (2000), Baca al., J. Biol. Chem. 272(16):10678-84 (1997), Roguska et al., Protein Eng. 9(10):895-904 (1996), Couto et al., Cancer Res. 55 (23 Supp):5973s -5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.) The antibodies that immunospecifically bind to integrin αᵥβ₃ can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

4.11.1 Polynucleotide Sequences Encoding an Antibody

The invention provides polynucleotides comprising a nucleotide sequence encoding an antibody or fragment thereof that immunospecifically binds to an antigen. The invention also encompasses polynucleotides that hybridize under high stringency, intermediate or lower stringency hybridization conditions, e.g., as defined supra, to polynucleotides that encode an antibody of the invention.

The polynucleotides may be obtained, and the nucleotide sequence of the polynucleotides determined, by any method known in the art. The nucleotide sequence of antibodies immunospecific for a desired antigen can be obtained, e.g., from the literature or a database such as GenBank. Since the amino acid sequences of VITAXIN™ is known, nucleotide sequences encoding this antibody can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3+ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et.al., eds., 1998, Current Protocols in Molecular Biology, John Wiley & Sons, N.Y., which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to a particular antigen. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

4.11.2 Recombinant Expression of Antibodies

Recombinant expression of an antibody that immunospecifically binds to an antigen requires construction of an expression vector containing a polynucleotide that encodes the antibody. Once a polynucleotide encoding an antibody molecule of the invention has been obtained, the vector for the production of the antibody molecule may be produced by recombinant DNA technology using techniques well-known in the art. See, e.g., U.S. Pat. No. 6,331,415, which is incorporated herein by reference in its entirety. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies which immunospecifically bind to one or more antigens is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NSO (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt- cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, N.Y. (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, N.Y. (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

The present invention also encompasses proteins, peptides and polypeptides of the invention produced by any method known in the art including, but not limited to, recombinant methods. See International Publication No. WO 02/070007, hereby incorporated by reference in its entirety.

Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

1. A method for managing, treating or ameliorating an inflammatory disease, an autoimmune disease, a disorder associated with abnormal bone metabolism, or cancer, or one or more symptoms thereof, said method comprising administering to a subject in need thereof a dose of an effective amount of VITAXIN® or an antigen-binding fragment thereof, or an antibody that competes with VITAXIN® for binding to integrin α_{V}β₃, and a dose of an effective amount of an HMG-CoA reductase inhibitor.

2. A method for managing, treating or ameliorating an inflammatory disease, an autoimmune disease, a disorder associated with abnormal bone metabolism, or cancer, or one or more symptoms thereof, said method comprising administering to a subject in need thereof a dose of an effective amount of VITAXIN® or an antigen-binding fragment thereof, or an antibody that competes with VITAXIN® for binding to integrin α_{V}β₃, and a dose of an effective amount of a bisphosphonate.

3. A method for managing, treating or ameliorating an inflammatory disease, an autoimmune disease, a disorder associated with abnormal bone metabolism, or cancer, or one or more symptoms thereof, said method comprising administering to a subject in need thereof a dose of an effective amount of VITAXIN® or an antigen-binding fragment thereof, or an antibody that competes with VITAXIN® for binding to integrin α_{V}β₃ conjugated or fused to an HMG-CoA reductase inhibitor or a bisphosphonate.

4. The method of claim 1 further comprising administering to said subject a dose of an effective amount of a bisphosphonate.

5. The method of claim 3 further comprising administering to said subject a therapy other than an integrin α_{V}β₃ antagonist, an HMG-CoA reductase inhibitor or a bisphosphonate.

6. The method of claim 1, 2, 3, 4, or 5 further comprising administering to said subject a therapy other than an integrin α_{V}β₃ antagonist, an HMG-CoA reductase inhibitor or a bisphosphonate.

7. The method of claim 5 or 6, wherein the therapy is an anti-inflammatory agent, immunomodulatory agent, an agent having a bone metabolism regulating agent, an anti-arthritic agent, or an anti-angiogenic agent.

8. The method of claim 7, wherein the bone metabolism regulating agent is calcitonin, Vitamin D, estrogen, or an estrogen receptor modulator.

9. The method of claim 1, 2, 3, 4, or 5, further comprising administering a dose of an effective amount of radiation therapy.

10. The method of claim 1, 2, 3, 4, or 5, wherein the cancer is prostate cancer, ovarian cancer, lung cancer, breast cancer, bone cancer, colon cancer, or melanoma.

11. The method of claim 1, 2, 3, 4, or 5, wherein the cancer has metastasized to the bone.

12. The method of claim 1, 2, 3, 4, or 5, wherein the inflammatory disease is arthritis, inflammatory arthritis, osteoarthritis or inflammatory osteolysis.

13. The method of claim 1, 2, 3, 4, or 5, wherein the autoimmune disorder is rheumatoid arthritis or Crohn's disease.

14. The method of claim 1, 2, 3, 4, or 5, wherein the disorder associated with aberrant bone metabolism is osteoporosis, aseptic loosening of a joint replacement, Paget's disease, periodontal disease, Behcet's disease or Gorham-Stout disease.

15. The method of claim 1, 2, 3, 4, or 5, wherein the cancer expresses integrin α_{V}β₃.

16. The method of claim 1, 2, 3, or 4, wherein the disorder associated with aberrant angiogenesis is vascular restenosis, diabetic retinopathy, macular degeneration or atherosclerosis.

17. The method of claim 1 or 4 further comprising administering to said subject one or more subsequent doses of an effective amount of one or more HMG-CoA reductase inhibitors.

18. The method of claim 2 or 4 further comprising administering to said subject one or more subsequent doses of an effective amount of one or more bisphosphonates.

19. The method of claim 1, 2, 3, 4 or 5 further comprising administering to said subject one or more subsequent doses of an effective amount of VITAXIN® or antigen-binding fragment thereof, or antibody that competes with VITAXIN® for binding to integrin α_{V}β₃.

20. The method of claim 1, 3, or 4, wherein at least one of the HMG-CoA reductase inhibitors is lovastatin, simvastatin, atorvastatin, pravastatin, fluvastatin, statin, cerivastatin, lescol, lupitor, rosuvastatin, atorvastatin or a pharmaceutically acceptable salt or mixture thereof.

21. The method of claim 2, 3, or 4, wherein at least one bisphosphonate is alendronate, cimadronate, clodronate, tiludronate, etidronate, ibandronate, neridronate, olpandronate, risedronate, piridronate, pamidronate, zolendronate or a pharmaceutically acceptable salt or mixture thereof.

22. The method of claim 1, 2 or 3, wherein the antibody that competes with VITAXIN® for binding to integrin α_{V}β₃ is not D12.

23. The method of claim 1, 2, 4, or 5, wherein the dose of VITAXIN® or an antigen-binding fragment thereof, or antibody that competes with VITAXIN® for binding to integrin α_{V}β₃ is administered parenterally, orally, intratumorally or intra-synovially.

24. The method of claim 1 or 4, wherein the dose of HMG-CoA reductase inhibitor is administered parenterally, orally, intratumorally or intra-synovially.

25. The method of claim 2 or 4, wherein the dose of bisphosphonate is administered parenterally, orally, intratumorally or intra-synovially.

26. The method of claim 3 or 5, wherein the dose of VITAXIN® or an antigen-binding fragment thereof, or antibody that competes with VITAXIN® for binding to integrin α_{V}β₃ conjugated or fused to a bisphosphonate or an HMG-CoA reductase inhibitor is administered parenterally, orally, intratumorally or intra-synovially.

27. The method of claim 1, 2, 3, 4, or 5, wherein the subject is human.
